(19) United States
(12) Patent Application Publication
(10) Pub. No.: US 2025/0319249 A1
(43) Pub. Date: Oct. 16, 2025

(54) CONTROLLING METHOD OF DRUG INJECTION DEVICE

(71) Applicant: EOFLOW CO., LTD., Seongnam-si Gyeonggi-do (KR)

(72) Inventors: Yongho JEON, Gwangmyeong-si Gyeonggi-do (KR); Seonhwan KIM, Seongnam-si Gyeonggi-do (KR); Youngjin JUNG, Yongin-si Gyeonggi-do (KR); Ha Bin IM, Yongin-si Gyeonggi-do (KR); Du Won JANG, Siheung-si Gyeonggi-do (KR)

(73) Assignee: EOFLOW CO., LTD., Seongnam-si Gyeonggi-do (KR)

(21) Appl. No.: 19/197,110

(22) Filed: May 2, 2025

Related U.S. Application Data
(63) Continuation-in-part of application No. 18/107,074, filed on Feb. 8, 2023.
Application No. 18/107,074 is a continuation of application No. PCT/KR2021/008629, filed on Jul. 7, 2021.
(63) Continuation-in-part of application No. PCT/KR2023/011277, filed on Aug. 2, 2023.
(63) Continuation-in-part of application No. PCT/KR2023/011278, filed on Aug. 2, 2023.
(63) Continuation-in-part of application No. PCT/KR2023/011279, filed on Aug. 2, 2023.
(63) Continuation-in-part of application No. PCT/KR2023/095093, filed on Nov. 21, 2023.

(30) Foreign Application Priority Data
Aug. 13, 2020 (KR) 10-2020-0101685
Nov. 3, 2022 (KR) 10-2022-0145126
Nov. 3, 2022 (KR) 10-2022-0145127
Nov. 3, 2022 (KR) 10-2022-0145128
Nov. 30, 2022 (KR) 10-2022-0163589
Nov. 14, 2023 (KR) 10-2023-0157580

(51) Int. Cl. A61M 5/172

(52) U.S. Cl. CPC A61M 5/172; A61M 2205/3584

(57) ABSTRACT

A method and apparatus for determining an inactive time of a medical liquid dose calculator are provided. According to the method, an infusion error of a medical liquid infusion device may be detected, and an inactive time of the medical liquid dose calculator may be determined in response to detection of the infusion error.

### CROSS-REFERENCE TO RELATED APPLICATIONS

This application is a continuation-in-part of U.S. patent application Ser. No. 18/107,074 which is a continuation of International Application No. PCT/KR2021/008629 filed on Jul. 7, 2021, which claims priority to Korean Patent Application No. 10-2020-0101685 filed on Aug. 13, 2020, International Application No. PCT/KR2023/011277 filed on Aug. 2, 2023, which claims priority to Korean Patent Application No. 10-2022-0145126 filed on Nov. 3, 2022, International Application No. PCT/KR2023/011278 filed on Aug. 2, 2023, which claims priority to Korean Patent Application No. 10-2022-01451267 filed on Nov. 3, 2022, International Application No. PCT/KR2023/011279 filed on Aug. 2, 2023, which claims priority to Korean Patent Application No. 10-2022-0145128 filed on Nov. 3, 2022, and International Application No. PCT/KR2023/095093 filed on Nov. 21, 2023, which claims priority to Korean Patent Application No. 10-2022-0163589 filed on Nov. 30, 2022 and Korean Patent Application No. 10-2023-0157580 filed on Nov. 14, 2023, the entire contents of which are herein incorporated by reference.

### TECHNICAL FIELD

The disclosure provides a method, device, and computer program product for determining an inactive time of a medical liquid dose calculator.

The disclosure provides a method of wireless communication connection with a medical liquid infusion device.

The disclosure provides a method of determining a medical liquid infusion amount in a medical liquid infusion device.

The disclosure provides a method of controlling a medical liquid infusion device.

The disclosure provides a method, apparatus, and computer program product for displaying a medical liquid infusion result when a wireless communication-related event occurs.

### BACKGROUND ART

Diabetes mellitus is a metabolic disorder that causes symptoms in which blood glucose levels are out of the normal range due to an insufficient secretion of insulin or a failure in its normal function. Diabetes is a complex disease that can affect each tissue of the human body due to complications such as blindness, renal failure, heart failure, and neuropathy, and the number of diabetic patients is reported to be increasing every year.

In the case of diabetes, it is necessary to measure blood sugar by using a blood glucose meter and control blood sugar through appropriate means such as diet, exercise programs, insulin injection, oral diabetes medicine, and the like.

Recently, when a medical liquid infusion device is discarded due to an abnormal reason, it is not possible to determine the exact amount of bolus actually injected into a user, and thus, a technique for solving this problem is required.

### SUMMARY

### Technical Problem

The disclosure provides a method, apparatus and computer program product for determining an inactive time of a medical liquid dose calculator. The technical objective to be achieved by the present embodiments is not limited to those described above, and other technical objectives can be inferred from the following embodiments.

The disclosure discloses various embodiments of a method of wireless communication connection with a medical liquid infusion device by using an information code.

The disclosure discloses various embodiments of a method of determining a medical liquid infusion amount in a medical liquid infusion device.

The disclosure discloses various embodiments of a method of controlling a medical liquid infusion device.

The disclosure discloses various embodiments of a method, apparatus, and a computer program product for displaying a medical liquid infusion result of a medical liquid infusion device when a wireless communication-related event occurs.

### Technical Solution to Problem

As a technical means for achieving the above-described technical objective, according to an aspect of the disclosure, there is provided is a method of determining an inactive time of a medical liquid dose calculator, the method including detecting an infusion error of a medical liquid infusion device, and determining an inactive time of the medical liquid dose calculator in response to the detecting of the infusion error.

According to another aspect of the disclosure, there is provided an apparatus for determining an inactive time of a medical liquid dose calculator, the apparatus including a memory storing at least one program, and a processor configured to perform calculations by executing the at least one program, wherein the processor is further configured to detect an infusion error of a medical liquid infusion device, and determine an inactive time of the medical liquid dose calculator in response to detection of the infusion error.

According to another aspect of the disclosure, there is provided a computer program product including a computer-readable recording medium having stored therein a program for executing a method, the method including detecting an infusion error of a medical liquid infusion device, and determining an inactive time of a medical liquid dose calculator in response to the detecting of the infusion error.

According to another aspect of the disclosure, there is provided a method of wireless communication connection with a medical liquid infusion device, the method including preparing a medical liquid infusion device on an outer surface of which an information code including an identifier is marked, reading, by a controller, the information code to recognize the identifier, transmitting, by the medical liquid infusion device upon a medical liquid being injected into the medical liquid infusion device, a pairing request signal that includes the identifier to its surroundings, receiving, by the controller, the pairing request signal and determining whether the identifier included in the pairing request signal matches the identifier recognized by using the information code, and, based on they matching, performing, by the controller, a wireless communication connection with the medical liquid infusion device that has transmitted the pairing request signal including the matching identifier.

According to another aspect of the disclosure, there is disclosed a method, performed by a processor, of determining a medical liquid infusion amount, the method including: preparing a next medical liquid infusion amount to be infused at a next time after a predetermined time period has elapsed from a reference time; receiving, at every unit time from the reference time until before the next time, a signal representing a measured temperature of a medical liquid stored in a medical liquid infusion device; calculating an efficacy reduction value for the medical liquid temperature measured at every unit time; calculating a compensatory infusion amount based on the calculated efficacy reduction value; and determining a corrected medical liquid infusion amount in which the calculated compensatory infusion amount is reflected.

According to another aspect of the disclosure, there is disclosed a method of controlling a medical liquid infusion device, the method including: establishing, by a medical liquid infusion device, which is configured to be attached to a subject and infuse a medical liquid, and a controller, a connection for first wireless communication; exchanging, by the controller and the medical liquid infusion device, an encryption key through second wireless communication; determining, by the controller, whether a received signal is an infusion signal; based on a result of the determining indicating that the signal is the infusion signal, encrypting, by the controller, the signal by using the encryption key; transmitting, by the controller, the encrypted signal to the medical liquid infusion device through the second wireless communication; decrypting, by the medical liquid infusion device, the encrypted signal by using the encryption key; and infusing, by the medical liquid infusion device, a medical liquid according to the decrypted signal.

According to another aspect of the disclosure, there is provided a method including: establishing, by a medical liquid infusion device and a controller, a connection for wireless communication; receiving, by the controller, a basic infusion program from a user; storing, by the controller, the received basic infusion program and transmitting the basic infusion program to the medical liquid infusion device through the wireless communication; performing, by the medical liquid infusion device, medical liquid infusion based on the received basic infusion program and transmitting, to the controller, medical liquid infusion information corresponding to the performed medical liquid infusion; generating and displaying, by the controller, a first graph corresponding to the received medical liquid infusion information; a wireless communication-related event occurring; and recognizing, by the controller, the wireless communication-related event, and generating and displaying a second graph corresponding to the stored basic infusion program, from a time point at which the event has occurred. In addition, various forms for implementing the disclosure may be provided.

### Advantageous Effects of Disclosure

According to the means for solving the problem of the disclosure, when a medical liquid infusion device is discarded due to an abnormal reason, by inactivating a medical liquid dose calculator for a certain period of time, the risk of over/under infusion of insulin may be prevented in advance.

### BRIEF DESCRIPTION OF DRAWINGS

FIG. 1 is a block diagram of an insulin management system including a user terminal, a controller, and a medical liquid infusion device;

FIGS. 2A to 2B are diagrams for explaining a method of setting a medical liquid dose calculator, according to an embodiment;

FIG. 3 is a diagram for explaining an example of use and disposal situations of a medical liquid infusion device according to an embodiment;

FIG. 4 is a diagram for explaining a case in which occlusion of an inlet of a medical liquid infusion device according to an embodiment has occurred blocked;

FIG. 5 is a flowchart of a method of calculating an inactive time of a medical liquid dose calculator when occlusion of an inlet of a medical liquid infusion device has occurred, according to an embodiment;

FIG. 6 is a diagram for explaining a case where a communication error occurs in a medical liquid infusion device according to an embodiment;

FIG. 7 is a flowchart of a method of calculating an inactive time of a medical liquid dose calculator when a communication error has occurred in a medical liquid infusion device, according to an embodiment; and

FIG. 8 is a flowchart of a method of determining an inactive time of a medical liquid dose calculator, according to an embodiment.

FIG. 9 is a conceptual diagram illustrating a medical liquid infusion management system according to another embodiment of the disclosure.

FIG. 10 illustrates a situation where a plurality of medical liquid infusion devices exist near a controller.

FIGS. 11A and 11B are perspective views illustrating an outer surface of a medical liquid infusion device according to an embodiment of the disclosure.

FIG. 12 is a block diagram illustrating a configuration of a medical liquid infusion device and a controller, according to an embodiment of the disclosure.

FIG. 13 is a flowchart illustrating a method of wireless communication connection with a medical liquid infusion device, according to an embodiment of the disclosure.

FIGS. 14 and 15 are explanatory diagrams illustrating some operations of a method of wireless communication connection with a medical liquid infusion device illustrated in FIG. 13.

FIG. 16 is a block diagram illustrating a configuration of a medical liquid infusion device and a controller, according to another embodiment of the disclosure.

FIG. 17 is a flowchart illustrating a method of wireless communication connection with a medical liquid infusion device, according to another embodiment of the disclosure.

FIG. 18 is a block diagram schematically illustrating a control module of a medical liquid infusion device, and constituent components associated with the control module.

FIG. 19 is a flowchart illustrating a method of determining a medical liquid infusion amount, according to an embodiment of the disclosure.

FIGS. 20A to 20C illustrate input/output screens of a controller for explaining a medical liquid infusion method according to a basic infusion program.

FIG. 21 is a graph illustrating a medical liquid infusion amount by time according to a basic infusion program of FIGS. 20A to 20C.

FIG. 22 is a graph illustrating a change in efficacy per hour according to temperature.

FIG. 23 is a graph illustrating a cumulative change in efficacy according to temperature and time.

FIG. 24 is a graph illustrating that the medical liquid infusion amount by time according to the basic infusion program of FIG. 21 has been compensated.

FIG. 25 is a flowchart illustrating a method of determining a medical liquid infusion amount, according to another embodiment of the disclosure.

FIG. 26 is a block diagram illustrating a configuration of a controller according to an embodiment of the disclosure.

FIG. 27 is a block diagram illustrating a schematic configuration of a controller according to an embodiment of the disclosure.

FIGS. 28A to 28C illustrate input/output screens of a controller for explaining a medical liquid infusion method according to a basic infusion program of the controller.

FIG. 29 is a graph illustrating an amount of medical liquid infused by time according to the basic infusion program of FIGS. 28A to 28C.

FIGS. 30A and 30B illustrate input/output module screens of a controller for explaining a medical liquid infusion method according to bolus injection of the controller.

FIGS. 31A and 31B are graphs illustrating an amount of medical liquid infused over time by the bolus injection of FIGS. 30A and 30B.

FIG. 32 is a block diagram schematically illustrating a control module of a medical liquid infusion device, and constituent components associated with the control module.

FIG. 33 is a flowchart illustrating a method of controlling a medical liquid infusion device, according to an embodiment of the disclosure.

FIG. 34 is a block diagram schematically illustrating a control module of a medical liquid infusion device, and constituent components associated with the control module.

FIG. 35 is a conceptual diagram schematically illustrating a flow of signals, data, and information that are received, transmitted, and generated by constituent components of the medical liquid infusion device of FIG. 34.

FIG. 36 is a block diagram schematically illustrating constituent components included in a controller that are associated with an embodiment of the disclosure.

FIG. 37 is a conceptual diagram schematically illustrating a flow of signals, data, and information that are received, transmitted, and generated by constituent components of the controller of FIG. 36.

FIG. 38 is a flowchart illustrating a method of displaying medical liquid infusion in a first period, according to an embodiment of the disclosure.

FIGS. 39A to 39C illustrate input/output screens of a controller for explaining a medical liquid infusion method according to a basic infusion program.

FIG. 40 illustrates a medical liquid infusion information chart displayed on a controller in a first period.

FIG. 41 is a flowchart illustrating a method of displaying medical liquid infusion in a second period, according to an embodiment of the disclosure.

FIG. 42 illustrates a medical liquid infusion information chart displayed on a controller in a second period.

FIG. 43 is a flowchart illustrating a method of displaying medical liquid infusion in a third period, according to an embodiment of the disclosure.

FIG. 44 illustrates a medical liquid infusion information chart displayed on a controller in a third period.

FIG. 45 is a flowchart illustrating a method of displaying medical liquid infusion in a first period, according to another embodiment of the disclosure.

FIG. 46 illustrates a screen of a controller for explaining a medical liquid infusion method according to an adjusted infusion command.

FIG. 47 illustrates a medical liquid infusion information chart displayed on a controller in a first period.

FIG. 48 is a flowchart illustrating a method of displaying medical liquid infusion in a second period, according to another embodiment of the disclosure.

FIG. 49 illustrates a medical liquid infusion information chart displayed on a controller in a second period.

FIG. 50 illustrates a medical liquid infusion information chart displayed on a controller in a third period.

FIG. 51 is a flowchart illustrating a method of displaying medical liquid infusion in a first period, according to yet another embodiment of the disclosure.

FIGS. 52A and 52B illustrate input/output screens of a controller for explaining a medical liquid infusion method according to an independent infusion command.

FIG. 53 illustrates a medical liquid infusion information chart displayed on a controller in a first period.

FIG. 54 is a flowchart illustrating a method of displaying medical liquid infusion in a second period, according to yet another embodiment of the disclosure.

FIG. 55 illustrates a medical liquid infusion information chart displayed on a controller in a second period.

FIG. 56 is a flowchart illustrating a method of displaying medical liquid infusion in a third period, according to yet another embodiment of the disclosure.

FIG. 57 illustrates a medical liquid infusion information chart displayed on a controller in a third period.

### DETAILED DESCRIPTION

A method and apparatus for determining an inactive time of a medical liquid dose calculator may be provided. According to the method of the disclosure, an infusion error of a medical liquid infusion device may be detected, and an inactive time of a medical liquid dose calculator may be determined in response to the detection of the infusion error.

### MODE OF DISCLOSURE

Hereinafter, embodiments of the disclosure will be described in detail so that those skilled in the art can easily practice the disclosure with reference to the accompanying drawings. However, the disclosure may be embodied in many different forms and is not limited to the embodiments described herein. And in order to clearly explain the disclosure in the drawings, parts irrelevant to the description are omitted, and similar reference numerals are attached to similar parts throughout the specification.

Throughout the specification, when a part is “connected” to another part, it may be construed that the part is “directly connected” but also the part is “electrically connected” to the other part with another element interposed therebetween. In addition, when a part “includes” a component, it does not mean that the part does not include components other than the mentioned component but may include other components provided that there is no indication to the contrary.

Hereinafter, the disclosure will be described in detail with reference to the accompanying drawings.

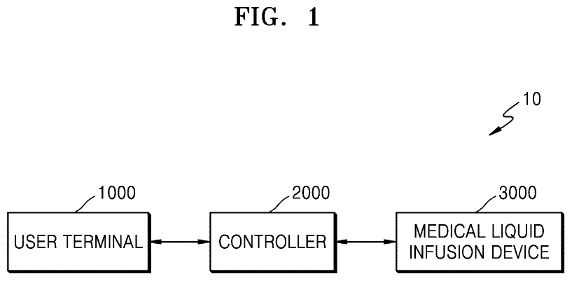

FIG. 1 is a block diagram of an insulin management system including a user terminal, a controller, and a medical liquid infusion device.

A user terminal 1000 refers to a communication terminal capable of using a web service in a wired or wireless communication environment. For example, the user terminal 1000 may include a smart phone, a tablet personal computer (PC), a PC, a smart TV, a mobile phone, a personal digital assistant (PDA), a laptop computer, a media player, a micro server, a global positioning system (GPS) device, an e-book reader, a terminal for digital broadcasting, a navigation device, a kiosk, an MP3 player, a digital camera, a home appliance, a camera-mounted device, and other mobile or non-mobile computing devices. Also, the user terminal 1000 may include a wearable device having a communication function and a data processing function, such as a watch, glasses, a hair band, and a ring. However, as described above, a terminal equipped with an application capable of internet communication may be used without limitation.

The user terminal 1000 may be connected with a pre-registered controller 2000 in a one-to-one manner. Also, the user terminals 1000 may receive data from the controller 2000 in order to prevent control by an external device. The user terminal 1000 may transmit setting information, for example, system time information to the controller 2000 within a preset range.

The controller 2000 performs a function of transmitting and receiving data to and from a medical liquid infusion device 3000, and may transmit a control signal related to infusion of a medical liquid, such as insulin, to the medical liquid infusion device 3000, and receive, from the medical liquid infusion device 3000, a control signal related to the measurement of a biometric value such as blood glucose.

The controller 2000 may transmit an instruction request to measure a current state of a user, to the medical liquid infusion device 3000, and receive measurement data from the medical liquid infusion device 3000 in response to the instruction request.

While the medical liquid infusion device 3000 performs a function of measuring the user's biometric values such as blood sugar level, blood pressure, heart rate, etc., but also a function of infusing a medical liquid to be infused into the user, such as insulin, glucagon, anesthetic, painkiller, dopamine, growth hormone, and smoking cessation aids.

The medical liquid infusion device 3000 may further include a storage unit that stores a substance to be periodically infused into a user, and may be controlled such that a dose to be infused is infused from the storage unit, according to an infusion signal generated by a controller.

Here, the medical liquid infusion device 3000 may transmit information such as measured values and infusion doses to the controller 2000. Selectively, the medical liquid infusion device 3000 may transmit a device status message, a biometric value measurement message, and a medical liquid infusion message to the controller 2000. For example, the medical liquid infusion device 3000 may transmit, to the controller 2000, a device status message including remaining battery capacity information of the device, whether the device boots successfully, and whether the infusion is successful. Messages delivered to the controller 2000 may be delivered to the user terminal 1000 via the controller 2000. Alternatively, the controller 2000 may transmit improved data obtained by processing received messages, to the user terminal 1000.

The medical liquid infusion device 3000 may also be implemented to communicate only with pre-registered controllers. In addition, in terms of hardware, the medical liquid infusion device 3000 may be classified into a measurement device that performs a function of measuring biometric values such as a user's blood sugar level, blood pressure, and heart rate, and an infusion device that performs a function of infusing a medical liquid such as insulin, glucagon, anesthetic, etc. That is, the measurement device and the infusion device may exist independently of each other. The controller 2000 may be connected to each of the infusion device and the measurement device to generate and provide a control signal for the infusion device based on a measurement value measured using the measurement device.

The user terminal 1000, the controller 2000, and the medical liquid infusion device 3000 may perform communication by using a network. For example, the network may include a Local Area Network (LAN), a Wide Area Network (WAN), a Value Added Network (VAN), a mobile radio communication network, a satellite communication network, and a mutual combination thereof, and refers to a comprehensive data communication network that allows each network constituent entity to communicate smoothly with each other, and may include wired Internet, wireless Internet, and mobile wireless communication networks. In addition, wireless communication may include, for example, wireless LAN (Wi-Fi), Bluetooth, Bluetooth low energy, Zigbee, Wi-Fi Direct (WFD), ultra wideband (UWB), infrared communication (IrDA, infrared Data Association), Near Field Communication (NFC), etc., but are not limited thereto.

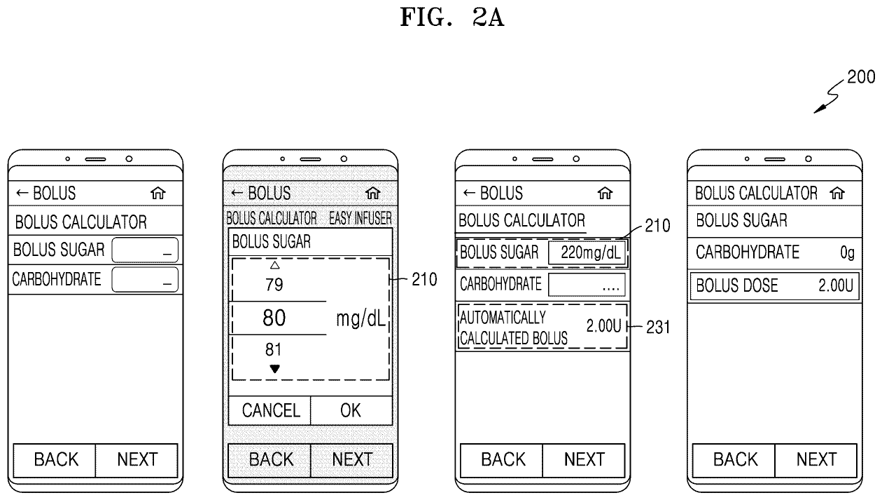

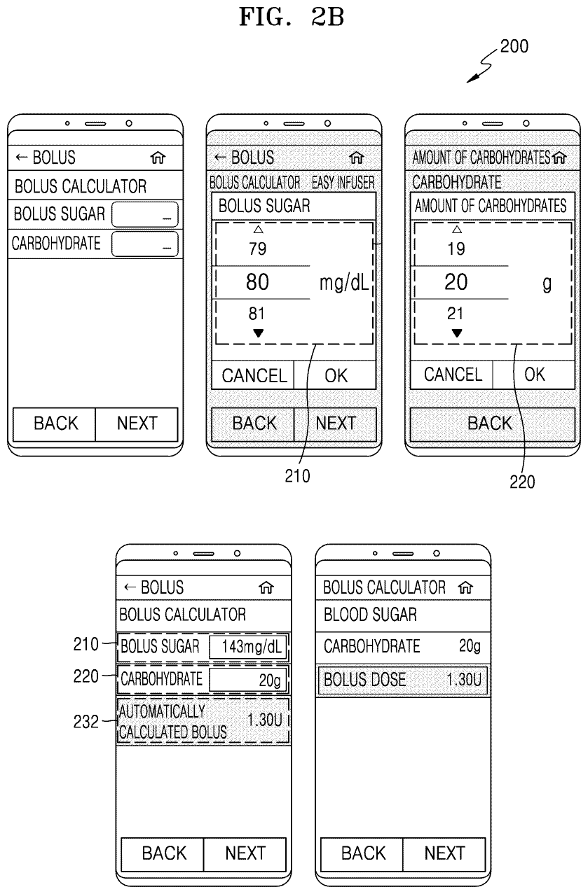

FIGS. 2A to 2B are diagrams for explaining a method of setting a medical liquid dose calculator, according to an embodiment.

A medical liquid dose calculator may be mounted in the user terminal 1000, the controller 2000, or the medical liquid infusion device 3000 of FIG. 1. The medical liquid dose calculator may include a calculator that calculates an amount of insulin to be infused into a user, separately from basic infusion. For example, the medical liquid dose calculator may calculate an amount of insulin needed to bring down blood sugar that rises from food or snacks. In addition, the medical liquid dose calculator may calculate an amount of insulin required to lower high blood sugar to a normal blood sugar range.

As described above, apart from the basal insulin infusion, insulin infused due to food intake or to lower high blood sugar may be referred to as a bolus. The medical liquid infusion calculator may be referred to as a bolus calculator, and the bolus calculator may calculate a bolus dose.

The bolus dose may be determined by various values. For example, the bolus dose may be determined based on a current blood glucose level, a carbohydrate-to-insulin ratio, a calibration coefficient, a target blood glucose level, and the amount of insulin remaining with activation time in the body among a previous bolus dose (Insulin On Board (IOB), Bolus on Board, or Active Insulin), a calibration threshold, an amount of activity, type and amount of ingested food, etc.

The calibration coefficient is a value indicating a blood glucose level that 1 unit of bolus insulin can lower. A range of the calibration coefficient is from about 1 mg/dl/U to about 400 mg/dl/U, and may be adjusted by 1 mg/dl/U. The calibration threshold may refer to a highest blood glucose level at which it is determined that insulin infusion is required to control blood sugar.

The bolus calculator may calculate a bolus dose based on the user's individual bolus profile set value, current blood sugar level, amount of ingested carbohydrates, and remaining body insulin level (IOB).

In detail, the bolus profile set value may be determined by a target blood glucose level, a carbohydrate-to-insulin ratio, a calibration coefficient, and insulin duration. The current blood glucose value refers to a blood glucose value measured within 10 minutes.

Referring to FIG. 2A, the user may input a current blood glucose value 210 into the medical liquid dose calculator. The user may omit the input of the amount of ingested carbohydrates. The user may input the current blood glucose value 210 in units of mg/dl. For example, the user may input 220 mg/dl as the current blood glucose value 210. The medical liquid dose calculator may calculate 2.00 U as a bolus dose 231 based on the current blood glucose value 210. A unit that may be input as the current blood glucose value 210 may be mmol/l, but is not limited to the above unit example.

Referring to FIG. 2B, the user may input the current blood glucose value 210 and an amount of ingested carbohydrates 220 into the medical liquid dose calculator. The user may input the current blood glucose value 210 in units of mg/dl and the amount of ingested carbohydrates 220 in units of g. For example, the user may input 220 mg/dl as the current blood glucose value 210 and input 20 g as the amount of ingested carbohydrate 220. The medical liquid dose calculator may calculate 1.30 U as the bolus dose 232 based on the current blood glucose value 210 and the amount of ingested carbohydrates 220.

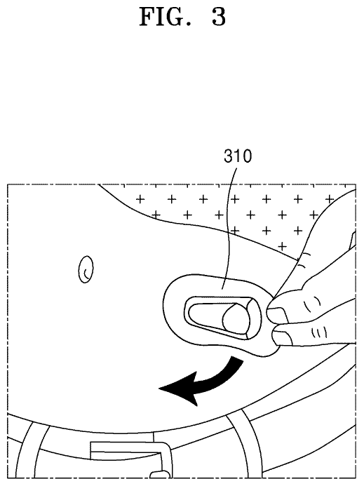

FIG. 3 is a diagram for explaining an example of use and disposal situations of a medical liquid infusion device according to an embodiment.

While being attached to the body of a user, a medical liquid infusion device 310 may infuse a medical liquid into the user. A certain amount of insulin is stored in a storage unit of the medical liquid infusion device 310.

In an embodiment, a medical liquid dose calculator may be mounted in a controller (not shown). The medical liquid infusion device 310 may transmit or receive data to or from the controller (not shown). The controller (not shown) may transmit bolus dose data calculated by the medical liquid dose calculator, to the medical liquid infusion device 310, and the medical liquid infusion device 310 may infuse insulin stored in the storage unit, to the user, based on the bolus dose.

In another embodiment, the medical liquid dose calculator may be mounted in the medical liquid infusion device 310. The medical liquid infusion device 310 may infuse the insulin stored in the storage unit into the user based on the bolus dose calculated by the medical liquid dose calculator.

The medical liquid infusion device 310 may be discarded for various reasons. For example, when the insulin stored in the storage unit is exhausted, or the use period of the medical liquid infusion device 310 expires, or an insulin infusion error is detected, the medical liquid infusion device 310 may be discarded.

In detail, when the insulin stored in the storage unit is less than a preset threshold value, the medical liquid infusion device 310 may be discarded. Also, when a preset period of time has elapsed after the operation of the medical liquid infusion device 310, the medical liquid infusion device 310 may be discarded.

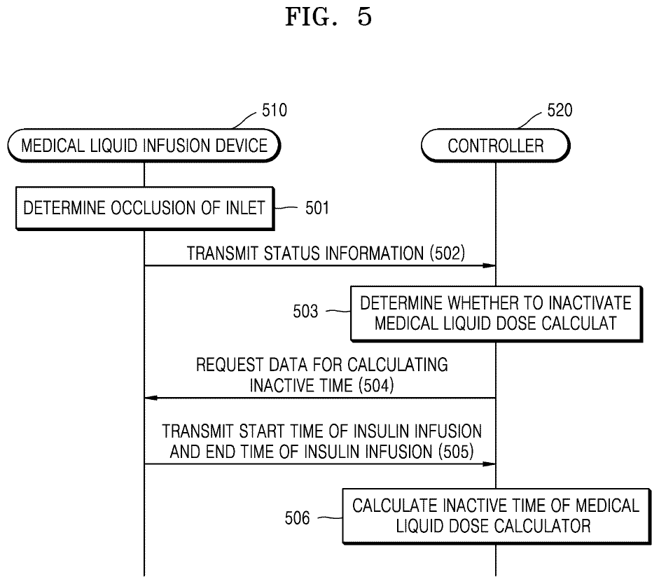

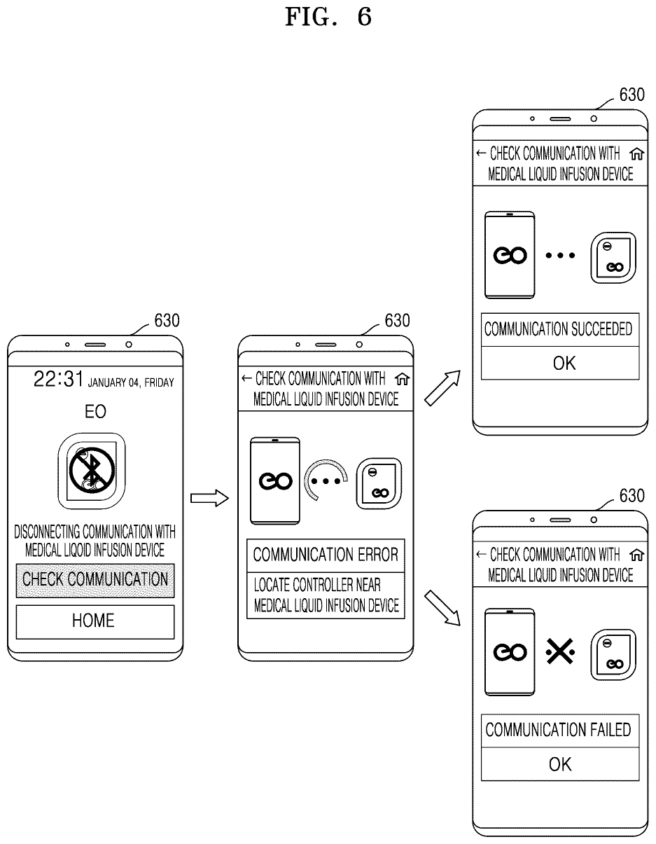

In addition, the medical liquid infusion device 310 may detect an infusion error in response to the occurrence of occlusion of an inlet of the medical liquid infusion device 310. An infusion error may be detected in response to a communication error occurring between the controller (not shown) and the medical liquid infusion device 310. A detailed description of a situation in which an infusion error occurs will be described later with reference to FIGS. 5 and 6.

Alternatively, when the user needs to dispose of the medical liquid infusion device 310, the medical liquid infusion device 310 may be discarded and detached from the body.

As described above with reference to FIGS. 2A and 2B, in order to calculate a bolus dose by the medical liquid dose calculator, the user needs to know the exact bolus amount that is actually infused. The medical liquid infusion device 310 may be discarded for various reasons. When the medical liquid infusion device 310 is discarded for normal reasons, the user may be able to identify the exact amount of actually infused bolus, whereas when the medical liquid infusion device 310 is discarded for an abnormal reason, the exact amount of actually infused bolus into the user may not be determined. Accordingly, when the medical liquid infusion device 310 is discarded due to an abnormal reason, the risk of over/under infusion of insulin may be prevented in advance by inactivating the medical liquid dose calculator for a certain period of time.

An inactive time of the medical liquid dose calculator may be determined according to the reason for discarding the medical liquid infusion device 310. When the medical liquid infusion device 310 is discarded for a normal reason, the medical liquid dose calculator may operate without a separate inactive time. For example, when the insulin stored in the storage unit is exhausted or the medical liquid infusion device 310 is discarded due to expiration of the use period of the medical liquid infusion device 310, the medical liquid dose calculator may operate without a separate inactive time.

When the medical liquid infusion device 310 is discarded due to an abnormal reason, the medical liquid dose calculator may operate after a certain inactive time. In detail, when an infusion error is detected in the medical liquid infusion device 310, the medical liquid dose calculator may operate after a certain inactive time. For example, when occlusion of an inlet of the medical liquid infusion device 310 has occurred or a communication error between a controller (not shown) and the medical liquid infusion device 310 has occurred, the medical liquid dose calculator may operate after a certain inactive time.

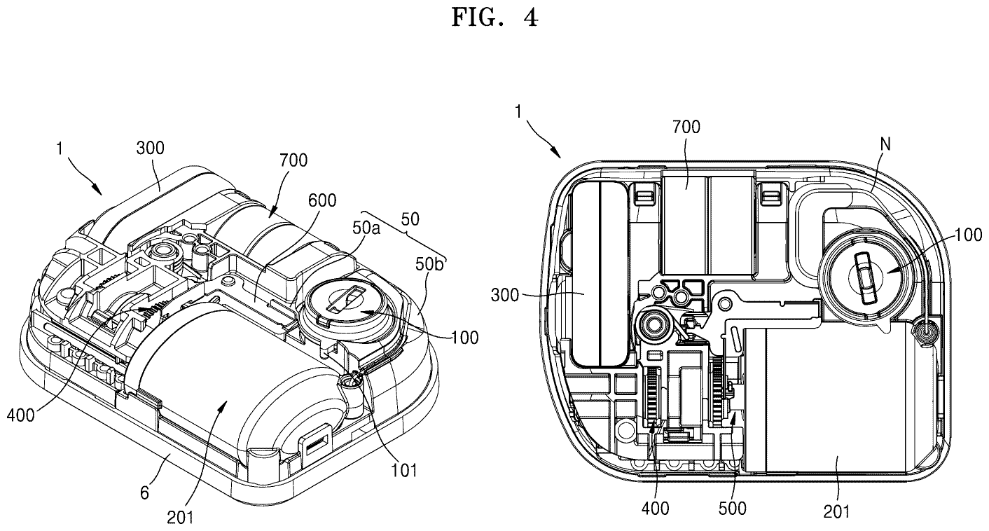

FIG. 4 is a diagram for explaining a case in which occlusion of an inlet of a medical liquid infusion device according to an embodiment has occurred.

Referring to FIG. 4, a medical liquid infusion device 1 may include a housing 5 covering the outside thereof, and an attachment portion 6 attached to the skin of a user. In the medical liquid infusion device 1, a plurality of parts are arranged in an inner space between the housing 5 and the attachment portion 6.

The medical liquid infusion device 1 may include a base body 50, a needle assembly 100, a storage unit 201, a driver 300, a driving unit 400, a clutch unit 500, a trigger member 600, and a battery 700.

The base body 50 forms a basic frame of the housing 5 and is mounted in an inner space of the housing 5. The base body 50 may be provided in plurality. In an embodiment, a first body 50a covering upper portions of internal components and a second body 50b covering lower portions of the internal components may be provided. The first body 50a and the second body 50b may be assembled to fix the internal components of the medical liquid infusion device 1 at preset positions. In another embodiment, the base body 50 may be formed as a single, integral frame.

The storage unit 201 is mounted on the base body 50 and fluidly connected to the needle assembly 100. Inside the storage unit 201, a plunger (not shown) may linearly move to discharge a medical liquid through a needle N.

The driver 300 may generate a driving force and transmit the driving force to the driving unit 400. The driving force transmitted by the driving unit 400 may linearly move a plunger (not shown) inside the storage unit 201 to discharge the medical liquid.

As the driver 300, all types of pumps having a medical liquid suction power and a medical liquid discharging power by electricity may be used. For example, all types of pumps such as a mechanical displacement type micropump and an electromagnetic motion type micropump may be used. The mechanical displacement type micropump is a pump that uses the movement of solids or fluids such as gears or diaphragms to create a pressure difference so as to induce a fluid flow, and examples thereof include diaphragm displacement pumps, fluid displacement pumps, and rotary pumps. The electromagnetic motion type micropump is a pump that uses energy in the form of electricity or magnetism directly to move a fluid, and examples thereof include an electrohydrodynamic pump (EHD), an electroosmotic pump, a magnetohydrodynamic pump, and an electro wetting pump.

When the driving unit 400 is engaged to the driver 300 by the clutch unit 500, the driver 300 rotates a driving wheel of the driving unit 400, and the rotation of the driving wheel may make a rod to linearly move and cause a plunger (not shown) to move inside the storage unit 201.

The driver 300 may include a membrane 320 disposed inside the cover 310. The membrane 320 may partition an inner space of the driver 300 into a first space S1 and a second space S2. The driver 300 may linearly move a driving shaft 330 by a change in volumes of the first space S1 and the second space S2.

On the other hand, in a process of infusing a medical liquid in the storage unit 201, into a patient through the needle N of the needle assembly 100, the inlet, which is a passage through which the medical liquid is infused into the patient, may be blocked due to various foreign substances. The inlet may include an inner passage of the needle N or a medical liquid transfer passage connecting the needle N to the storage unit 201, but is not limited thereto. When the inlet is blocked due to various foreign substances, a necessary amount of medical liquid may not be infused into the patient. Even worse, the medical liquid may not be infused into the patient at all.

In an embodiment, the medical liquid infusion device 1 may determine whether or not the inlet is blocked, based on a driving time of the driver 300. For example, when the driving time of the driver 300 exceeds a threshold value, it may be determined that the inlet is blocked.

According to various embodiments of the disclosure, the medical liquid infusion device 1 may determine whether an occlusion has occurred in a medical liquid discharge path due to foreign substances or the like, and notify the user of the occlusion and that a problem has occurred in the medical liquid infusion device 1.

When a control unit of the medical liquid infusion device 1 determines that occlusion has occurred in the inlet of the medical liquid infusion device 1, the control unit may transmit, to a notification unit, status information indicating that the occlusion has occurred. The notification unit may receive the status information from the control unit and express that the occlusion has occurred, to the outside. For example, the notification unit may output a sound indicating that occlusion has occurred or transmit, to an external device, information indicating that occlusion has occurred.

The user of the medical liquid infusion device 1 may discard the medical liquid infusion device 1 after receiving notification that occlusion in the inlet of the medical liquid infusion device 1 has occurred, through the notification unit. Discarding of the medical liquid infusion device 510 due to occlusion of the inlet of the medical liquid infusion device 1 corresponds to discarding due to abnormal reasons.

In an embodiment, the medical liquid infusion device 1 may transmit, to a controller (not shown), status information indicating that occlusion of the inlet has occurred. The controller (not shown) may detect an infusion error in response to receiving the status information indicating that occlusion of the inlet has occurred, and inactivate a medical liquid dose calculator mounted in the controller (not shown) for a certain period of time. In another embodiment, the medical liquid dose calculator may be mounted on the medical liquid infusion device 1.

FIG. 5 is a flowchart of a method of calculating an inactive time of a medical liquid dose calculator when occlusion of an inlet of a medical liquid infusion device has occurred, according to an embodiment.

Referring to FIG. 5, a medical liquid infusion device 510 and a controller 520 may communicate with each other. The controller 520 may include a component included in a user terminal such as a smart phone or a PC, or may include a component independent of the user terminal. While the controller 520 communicating with the medical liquid infusion device 510 is illustrated in FIG. 5, the controller 520 may be a component included in the medical liquid infusion device 510. Hereinafter, it is assumed that the medical liquid infusion device 510 and the controller 520 perform communication using a network.

The medical liquid dose calculator may be mounted in the controller 520. The medical liquid dose calculator may include a calculator that calculates an amount of insulin to be infused into a user, separately from basic infusion. For example, the medical liquid dose calculator may calculate an amount of insulin needed to bring down blood sugar that rises from food or snacks. In addition, the medical liquid dose calculator may calculate an amount of insulin required to lower high blood sugar to a normal blood sugar range.

While being attached to the body of a user, the medical liquid infusion device 510 may infuse a medical liquid into the user. In addition, the medical liquid infusion device 510 may be discarded for various reasons. In detail, the medical liquid infusion device 510 may be discarded due to normal or abnormal reasons. When the medical liquid infusion device 510 is discarded for a normal reason, the exact amount of bolus that is actually infused into the user may be identified, and thus, and the medical liquid dose calculator may operate without inactive time. On the other hand, when the medical liquid infusion device 510 is discarded due to an abnormal reason, it is difficult to determine the exact amount of bolus actually infused into the user, and thus, the medical liquid dose calculator needs to be inactivated for a certain period of time.

In operation 501, the medical liquid infusion device 510 may determine that occlusion in the inlet has occurred. For example, the medical liquid infusion device 510 may determine whether or not occlusion in the inlet has occurred, based on a driving time of a driver that drives a pump. Alternatively, the medical liquid infusion device 510 may determine that occlusion in the inlet has occurred, in response to detection of a decrease in a current flowing through the pump. The occlusion of the inlet is an infusion error and corresponds to an abnormal reason among the reasons for discarding the medical liquid infusion device 510.

In operation 502, the medical liquid infusion device 510 may transmit, to the controller 520, status information indicating that occlusion in the inlet has occurred.

In operation 503, the controller 520 may determine whether to inactivate the medical liquid dose calculator. In detail, the controller 520 may determine to inactivate the medical liquid dose calculator in response to receiving status information indicating that occlusion in the inlet has occurred.

In operation 504, the controller 520 may request data for calculating the inactive time from the medical liquid infusion device 510.

In operation 505, the medical liquid infusion device 510 may transmit information about a start time of insulin infusion and an end time of insulin infusion, to the controller 520.

In operation 506, the controller 520 may calculate the inactive time of the medical liquid dose calculator. The controller 520 may receive information about the start time of insulin infusion and the end time of insulin infusion, and calculate a reference time for insulin infusion based on the star time of insulin infusion and the end time of insulin infusion. In addition, the controller 520 may acquire a current time and preset insulin duration.

In an embodiment, the controller 520 may determine an inactive time of the medical liquid dose calculator, based on the current time, the preset insulin duration, and the reference time for insulin infusion.

For example, when the start time of insulin infusion is 13:00 PM and the end time of insulin infusion is 14:00 PM, the controller 520 may calculate an average value of the start time of insulin infusion and the end time of insulin infusion as the reference time for insulin infusion. That is, the reference time for insulin infusion is 13:30 PM.

The controller 520 may determine the inactive time by adding the insulin duration to the reference time for insulin infusion and subtracting the current time from the reference time for insulin infusion. For example, when the reference time for insulin infusion is 13:30 PM, the insulin duration is 5 hours, and the current time is 15:00 PM, the controller 520 may set the inactive time of the medical liquid dose calculator to 3 hours and 30 minutes. That is, the medical liquid dose calculator is inactivated until 18:30 PM, which is 3 hours and 30 minutes from the current time (15:00 PM).

Meanwhile, referring to operations 504 to 505 of FIG. 5, the controller 520 receiving information about the start time of insulin infusion and the end time of insulin infusion from the medical liquid infusion device 510 is described. However, in another embodiment, information about the start time and the end time of insulin infusion may be information stored in the controller 520. In this case, the controller 520 may calculate the reference time for insulin infusion based on the stored start time of insulin infusion and the stored end time of insulin infusion, without requesting additional information, and may also calculate an inactive time of the medical liquid dose calculator.

FIG. 6 is a diagram for explaining a case where a communication error occurs in a medical liquid infusion device, according to an embodiment.

A controller 610 and a medical liquid infusion device 620 may perform communication by using a network. For example, the network may include a LAN, a WAN, a VAN, a mobile radio communication network, a satellite communication network, and a mutual combination thereof, and refers to a comprehensive data communication network that allows each network constituent entity to communicate smoothly with each other, and may include wired Internet, wireless Internet, and mobile wireless communication networks. In addition, wireless communication may include, for example, wireless LAN (Wi-Fi), Bluetooth, Bluetooth low energy, Zigbee, WFD, UWB, infrared communication (IrDA, infrared Data Association), NFC, etc., but are not limited thereto.

A communication state between the controller 610 and the medical liquid infusion device 620 may be displayed through a user terminal 630. This is merely an example, and a communication state between the controller 610 and the medical liquid infusion device 620 may also be displayed through a display of the controller 610 or the medical liquid infusion device 620.

When a communication error occurs between the controller 610 and the medical liquid infusion device 620, the medical liquid infusion device 620 may be discarded. Discarding of the medical liquid infusion device 620 due to a communication error between the controller 610 and the medical liquid infusion device 620 corresponds to discarding due to abnormal reasons.

In an embodiment, the controller 610 may detect an infusion error in response to the occurrence of a communication error, and inactivate a medical liquid dose calculator mounted in the controller 610 for a certain period of time. In another embodiment, the medical liquid dose calculator may be mounted on the medical liquid infusion device 620.

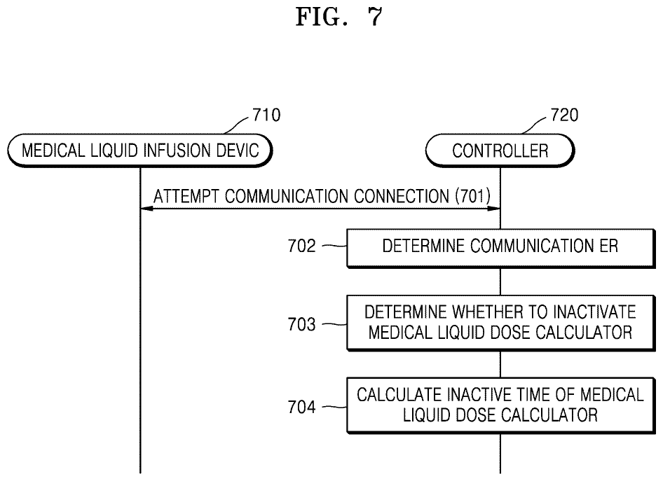

FIG. 7 is a flowchart of a method of calculating an inactive time of a medical liquid dose calculator when a communication error occurs in a medical liquid infusion device, according to an embodiment.

Referring to FIG. 7, a medical liquid infusion device 710 and a controller 720 may communicate with each other. Descriptions of the medical liquid infusion device 710 and the controller 720 correspond to those provided with reference to FIG. 5, and thus will be omitted here.

While being attached to the body of a user, the medical liquid infusion device 710 may infuse a medical liquid into the user. In addition, the medical liquid infusion device 710 may be discarded for various reasons. In detail, the medical liquid infusion device 710 may be discarded due to normal or abnormal reasons. When the medical liquid infusion device 710 is discarded for a normal reason, the exact amount of bolus that is actually infused into the user may be identified, and thus, and the medical liquid dose calculator may operate without inactive time. On the other hand, when the medical liquid infusion device 710 is discarded due to an abnormal reason, it is difficult to determine the exact amount of bolus actually infused into the user, and thus, the medical liquid dose calculator needs to be inactivated for a certain period of time.

In operation 701, the medical liquid infusion device 710 and the controller 720 may attempt a communication connection with each other.

In operation 702, the controller 720 may determine that a communication error has occurred, when communication with the medical liquid infusion device 710 fails. In detail, the controller 720 may continuously attempt a communication connection with the medical liquid infusion device 710, and as a result, if the communication connection fails a certain number of times or more, the controller 720 may determine that a communication error has occurred.

In operation 703, the controller 520 may determine whether to inactivate the medical liquid dose calculator. In detail, the controller 720 may determine to inactivate the medical liquid dose calculator in response to the occurred communication error.

In operation 704, the controller 720 may calculate an inactive time of the medical liquid dose calculator. In detail, the controller 720 may acquire a current time and preset insulin duration. In addition, the controller 720 may determine, as a communication error occurrence time, a time when it is determined that a communication error occurs in operation 702.

In an embodiment, the controller 720 may determine the inactive time of the medical liquid dose calculator, based on the current time, the preset insulin duration, and the communication error occurrence time.

For example, when the communication error occurrence time is 14:00 PM, the insulin duration is 5 hours, and the current time is 15:00 PM, the controller 720 may set the inactive time of the medical liquid dose calculator to 4 hours. That is, the medical liquid dose calculator is inactivated until 19:00 PM, which is 4 hours from the current time (15:00 PM).

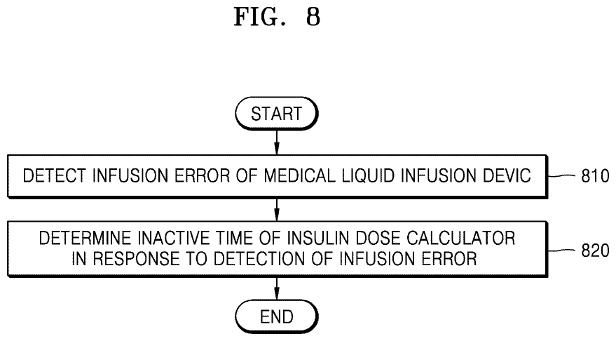

FIG. 8 is a flowchart of a method of determining an inactive time of a medical liquid dose calculator, according to an embodiment.

In an embodiment, the medical liquid dose calculator may be mounted in a controller. A medical liquid infusion device may transmit or receive data to or from the controller. The controller may transmit bolus dose data calculated by the medical liquid dose calculator, to the medical liquid infusion device, and the medical liquid infusion device may infuse insulin stored in a storage unit, to the user based on the bolus dose. The controller may include a component included in a user terminal such as a smart phone or a PC, or may include a component independent of the user terminal.

In another embodiment, the medical liquid dose calculator may be mounted in the medical liquid infusion device. The medical liquid infusion device may infuse insulin stored in the storage unit, into the user, based on the bolus dose calculated by the medical liquid dose calculator.

Hereinafter, it is assumed that the medical liquid dose calculator is mounted in the controller.

Referring to FIG. 8, in operation 810, the controller may detect an infusion error of the medical liquid infusion device.

In an embodiment, the controller may detect an infusion error in response to occlusion of an inlet of the medical liquid infusion device.

In another embodiment, the controller may detect an infusion error in response to a communication error with respect to the medical liquid infusion device.

In operation 820, the controller may determine an inactive time of the medical liquid dose calculator in response to detection of the infusion error.

When the controller detects an infusion error due to occlusion of the medical liquid infusion device, the controller may calculate a reference time for insulin infusion based on a start time of insulin infusion and an end time of insulin infusion. In addition, the controller may determine an inactive time based on a current time, preset insulin duration, and the reference time for insulin infusion.

When the controller detects an infusion error due to a communication error with respect to the medical liquid infusion device, the controller may obtain a communication error occurrence time when the communication error occurred. In addition, the controller may determine the inactive time based on the current time, the preset insulin duration, and the communication error occurrence time.

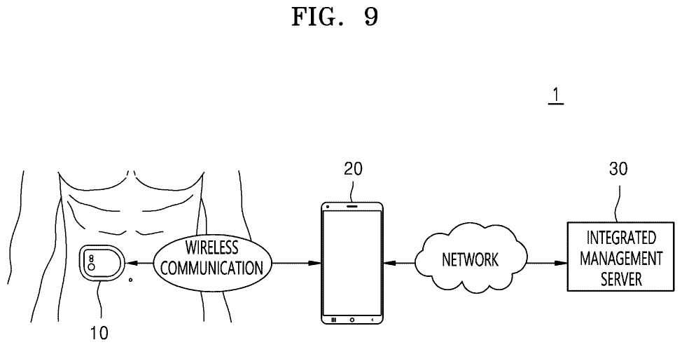

The disclosure discloses various embodiments of a method of wireless communication connection with a medical liquid infusion device by using an information code. Hereinafter, descriptions will be provided with reference to FIGS. 9 to 17.

FIG. 9 is a conceptual diagram illustrating a medical liquid infusion management system 1 according to an embodiment of the disclosure.

The medical liquid infusion management system 1 may include a medical liquid infusion device 10, a controller 20, and an integrated management server 30.

The medical liquid infusion device 10 may be attached to a medical liquid infusion subject, and may measure biometric values of the user, such as a blood glucose level, a blood pressure, or a heart rate, and infuse a medical liquid stored therein into the subject in a preset dose. In an alternative embodiment, the medical liquid infusion device 10 may be mounted on the user's body. In another alternative embodiment, the medical liquid infusion device 10 may also be mounted on an animal to infuse a medical liquid into the animal.

The medical liquid infusion device 10 may include a storage unit that stores a medical liquid to be periodically infused into the user, and may be controlled according to an infusion signal generated by the controller 20 such that the medical liquid is infused from the storage unit into the user. For example, the medical liquid may include an insulin-based medical liquid for a patient with diabetes, and may be of various types such as glucagon, an anesthetic, a painkiller, dopamine, a growth hormone, a smoking cessation aid, or a cardiac medical liquid.

The medical liquid infusion device 10 may be connected to the controller 20 in a one-to-one (point-to-point) manner through a wireless communication network. Here, the wireless communication network may be Bluetooth or Bluetooth Low Energy, and the following description will be based on this. However, the disclosure is not limited thereto, and Zigbee, wireless LAN (Wi-Fi), WFD, UWB, IrDA, and NFC may also be used.

The controller 20 may perform a wireless communication connection with the medical liquid infusion device 10, determine whether the medical liquid infusion device 10 is in an ineligible state, transmit and receive data to and from the medical liquid infusion device 10, transmit a control signal related to infusion of a medical liquid to the medical liquid infusion device 10, receive information about measurement of a biometric value such as blood glucose from the medical liquid infusion device 10, and monitor a usage status of the medical liquid infusion device 10. For example, the controller 20 may monitor an amount of medical liquid infused from the medical liquid infusion device 10, a number of medical liquid infusions, an amount of medical liquid stored in the storage unit, and bio-information about a user, and based on the monitored information, the user may operate the medical liquid infusion device 10 through the controller 20.

The controller 20 refers to a communication terminal capable of using an application in a communication environment. Here, the controller 20 may be a portable terminal of the user. In more detail, the controller may include any form of a smart remote controller, any form of a smart phone, a computer (e.g., a desktop computer, a laptop computer, or a tablet computer), a handheld computing device (e.g., a PDA or an email client), a form of a wearable device that may be attached to or mounted on a user's body for use, or any form of another type of computing or communication platform, but the disclosure is not limited thereto.

The controller 20 may be connected to the integrated management server 30 through a network. Here, the network may be a wireless communication network, and may be, for example, a mobile radio communication network, a wireless LAN, or Wi-Fi.

The integrated management server 30 allows a user, a guardian, and medical staff to remotely control the controller 20 and the medical liquid infusion device 10 through a web-based or application-based platform. The integrated management server 30 may perform data synchronization with the controller 20. A third party other than the user, such as a guardian or medical staff, may remotely transmit an infusion signal to the medical liquid infusion device 10 through the integrated management server 30. Obviously, in this case, to maintain security, instead of the integrated management server 30 directly transmitting the infusion signal to the medical liquid infusion device 10, a method may be used wherein the integrated management server 30 transmits the infusion signal to the controller 20, and the controller 20 then transmits the infusion signal to the medical liquid infusion device 10. In addition, data may be transmitted and received to and from the integrated management server 30 and the controller 20. The integrated management server 30 may receive, through a web or an application, information about a medical liquid, such as a type of medical liquid or a cumulative time the medical liquid has been stored in the storage unit, or information about a user, such as a site where the medical liquid infusion device is attached, a past absorption rate of a medical liquid for each site where the medical liquid infusion device was attached, a biometric value of the user that is input through the controller 20, or emotional stress information about the user that is input through the controller 20, and may transmit the received information to the controller 20. The integrated management server 30 may receive and store device data from the controller 20, and may receive and store measured biometric values. In addition, the integrated management server 30 may generate, manage, and analyze statistical pathological data by using stored medical liquid infusion history and biometric values, and provide the data in a report form to the user, a guardian, and medical staff.

Meanwhile, the medical liquid infusion device 10 and the controller 20 need to be connected exclusively in a one-to-one manner to transmit and receive data. When a single controller 20 controls a plurality of medical liquid infusion devices 10, or when a plurality of controllers 20 control a single medical liquid infusion device 10, confusion may occur due to a plurality of control signals, a security problem may arise, and this may pose a serious threat to a user's life. Thus, when the controller 20 and the medical liquid infusion device 10 pair with each other to communicate by using the above-described wireless communication method, the controller 20 uses a method of automatically pairing with only one nearby medical liquid infusion device 10 that has the highest pairing request signal strength. That is, conditions for automatic pairing are determined based on the distance between the medical liquid infusion device 10 and the controller 20, and the strength of a pairing request signal from the medical liquid infusion device 10.

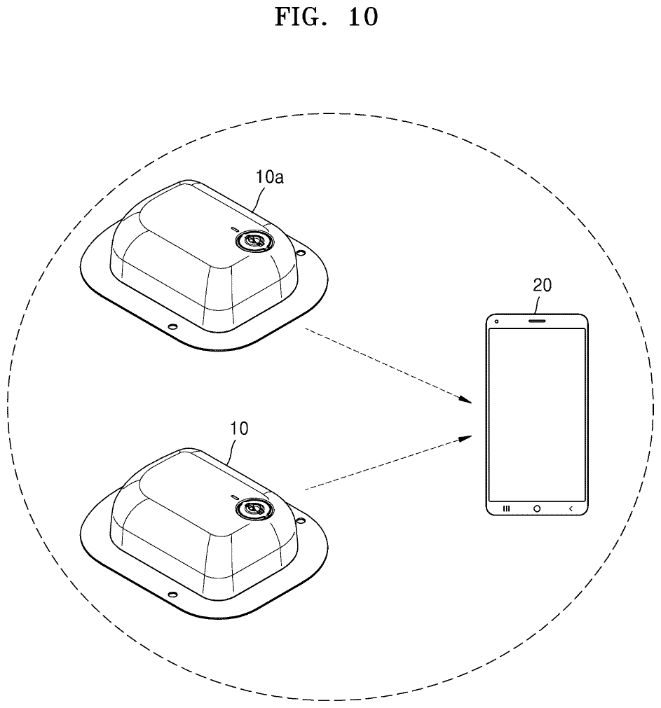

FIG. 10 illustrates a situation where a plurality of medical liquid infusion devices 10 and 10a exist near the controller 20.

Referring to FIG. 10, a plurality of medical liquid infusion devices 10 and 10a may exist around the controller 20.

Here, ‘around’ may refer to an area within the maximum reach of a signal for a wireless communication connection, for example, about 1 m. In this case, the controller 20 may receive pairing request signals with very similar strengths from the plurality of medical liquid infusion devices 10 and 10a. The controller 20 may compare the strengths of the plurality of pairing request signals to automatically pair with the one medical liquid infusion device that has the highest pairing request signal strength.

However, such a method of automatically pairing by using only the device's distance and signal strength has a problem in that the controller 20 pairs with a non-target medical liquid infusion device 10a, with which the user does not want to pair, instead of the target medical liquid infusion device 10, with which the user wants to pair. For example, when the user has accidentally or unintentionally placed a non-target medical liquid infusion device 10a near the controller 20, the non-target medical liquid infusion device 10a may be automatically paired with the controller 20, instead of the target medical liquid infusion device 10. In such a case, the inconvenience arises that the user has to discard the non-target medical liquid infusion device 10a and then perform pairing between the target medical liquid infusion device 10 and the controller 20 again. Furthermore, when the user attaches the target medical liquid infusion device 10 to the user's body while being unaware that the non-target medical liquid infusion device 10a has been paired with the controller 20, a problem may occur where the user does not appropriately receive a medical liquid infusion at a necessary time.

Meanwhile, in a case in which the controller 20 performs automatic pairing by using only the device's distance and signal strength, a process of checking in advance whether the target medical liquid infusion device 10 is in an ineligible state does not exist. Here, an ineligible state may refer to a case in which the target medical liquid infusion device 10 is a model or version that cannot interwork with the controller 20, cannot be controlled by the controller 20, or cannot establish a wireless communication connection with the controller 20, or is a device to which a regional restriction is applied. When the target medical liquid infusion device 10 is in an ineligible state and is automatically paired with the controller 20, the target medical liquid infusion device 10 cannot be used through the controller 20. Thus, a loss occurs from having to forcibly discard the target medical liquid infusion device 10, and unnecessary waste of medical liquid already injected into the target medical liquid infusion device 10 also occurs.

Embodiments of the disclosure disclose a method of wireless communication connection for pairing, by using an information code, the target medical liquid infusion device 10 that the user desires, with the controller 20, even when a plurality of medical liquid infusion devices 10 and 10a exist around the controller 20. In addition, another embodiment of the disclosure discloses a method of wireless communication connection for preventing pairing with a medical liquid infusion device 10 that is in an ineligible state, by determining, based on an information code, a state of the medical liquid infusion device 10 before the controller 20 pairs with the medical liquid infusion device 10.

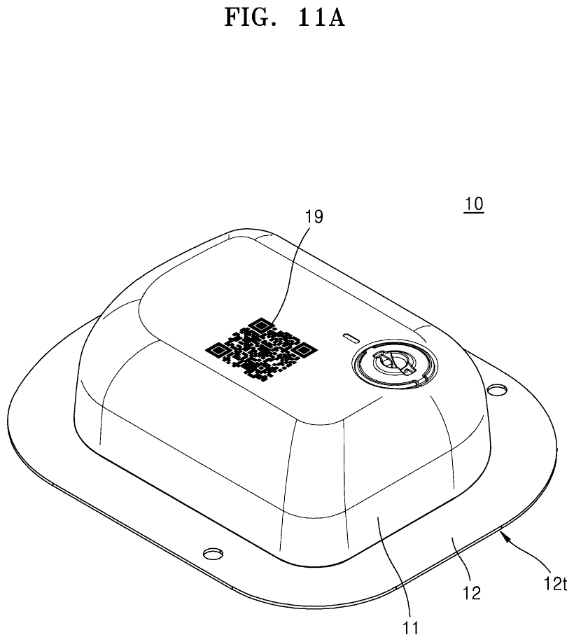

FIGS. 11A and 11B are perspective views illustrating an outer surface of the medical liquid infusion device 10 according to an embodiment of the disclosure.

Referring to FIGS. 11A and 11B, the medical liquid infusion device 10 according to an embodiment of the disclosure may include a housing 11 that forms an exterior appearance and covers an outside thereof, and an attachment portion 12 arranged on a lower surface of the housing 11 and positioned adjacent to a user's skin.

The housing 11 is made of plastic, and an upper surface of the housing 11 faces a direction opposite to the user's body when the medical liquid infusion device 10 is attached to the user.

The attachment portion 12 may include an adhesive portion (not shown) and a protective tape 12t. The adhesive portion (not shown) may have applied thereto an adhesive tape, an adhesive, or the like, such that it may adhere to the user's skin. One surface of the protective tape 12t has a smooth coating, is in direct contact with the adhesive portion, and serves to maintain and protect an adhesive capability of the adhesive portion until the adhesive portion adheres to the user's skin. The other surface of the protective tape 12t is exposed to the outside of the medical liquid infusion device 10, and on the other surface, a medical liquid injection port for injecting a medical liquid with a syringe into a storage unit of the medical liquid infusion device is indicated (by an arrow), and a needle cover assembly 700n is arranged. A needle assembly (not shown) may be mounted below the needle cover assembly 700n. The needle cover assembly 700n may protect the needle assembly and may prime air stored in the storage unit before the medical liquid is injected.

According to an embodiment of the disclosure, an information code 19 may be attached to an outer surface of the medical liquid infusion device 10.

Here, the information code 19 is a code in which two-dimensional (2D) pixels are arranged to store information that may be read by a reader, and may be, for example, in the form of a 2D code such as a barcode, a Quick Response (QR) code, or a data matrix code. However, the disclosure is not limited thereto, the information code 19 may also be in the form of a pin number or key information.

In addition, the information code 19 may include a unique identifier capable of identifying the medical liquid infusion device 10 when a wireless communication connection is established between the medical liquid infusion device 10 and the controller 20. Here, the identifier includes unique identification information of the medical liquid infusion device 10 and may include, for example, at least one of a unique name, a serial number, and a unique wireless communication address of the medical liquid infusion device 10.

According to the embodiment of FIG. 11A, a position where the information code 19 is attached may be an upper surface of the housing 11 of the medical liquid infusion device 10. According to the embodiment of FIG. 11B, a position where the information code 19 is attached may be another surface of the protective tape 12t of the attachment portion 12 of the medical liquid infusion device 10.

The medical liquid infusion device 10 is contained in a protective case (not shown) for product protection and hygiene, and is then distributed to a user. The information code 19 is marked on the medical liquid infusion device 10 inside the protective case, and thus is not exposed to the outside until the protective case is opened. Thus, because the information code 19 is directly marked on the outer surface of the medical liquid infusion device 10, it has the advantage of facilitating security maintenance. Furthermore, when the information code 19 is marked on another surface of the protective tape 12t as in the embodiment of FIG. 11B, the information code 19 is discarded upon removal of the protective tape 12t, and thus, there is no possibility of reusing an already used medical liquid infusion device 10 in conjunction with the controller 20. Thus, this has the effect of eliminating concerns about the reuse of a discarded medical liquid infusion device 10.

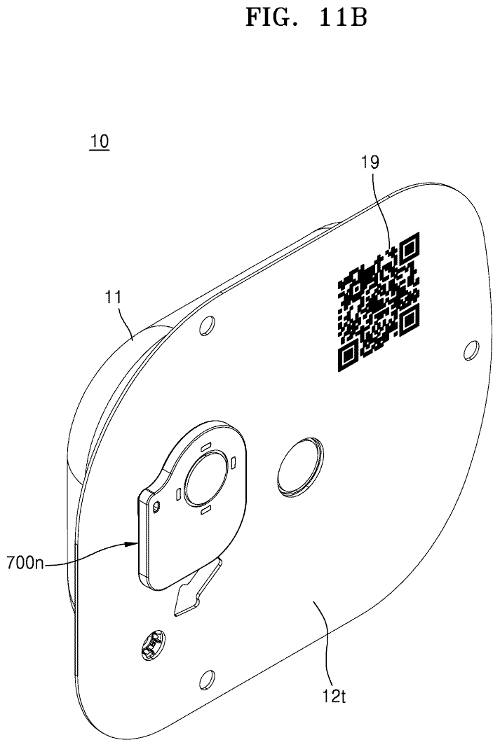

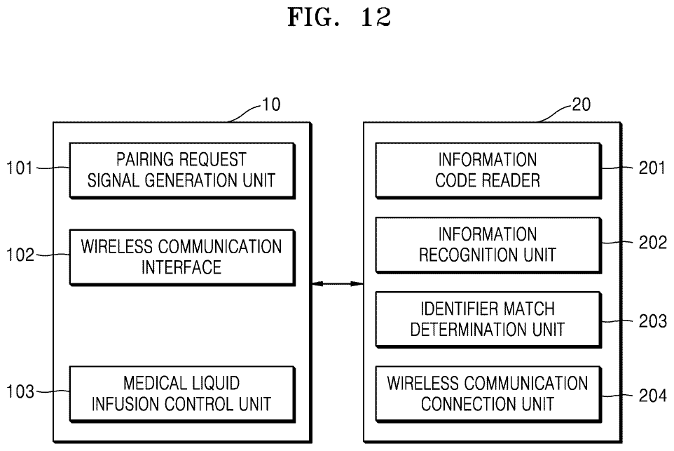

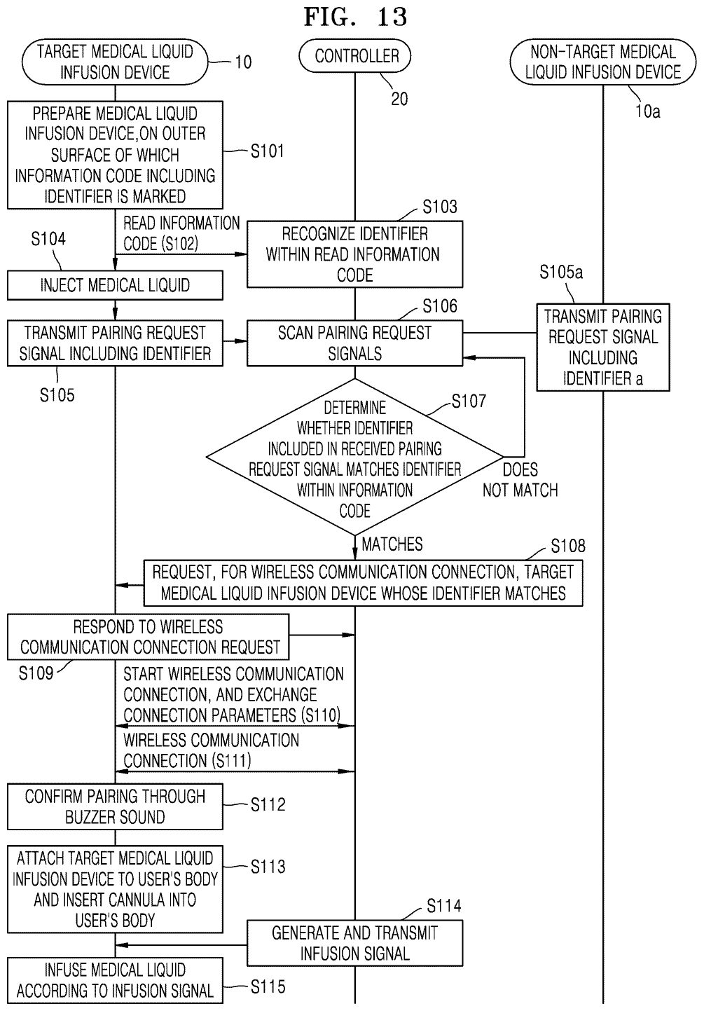

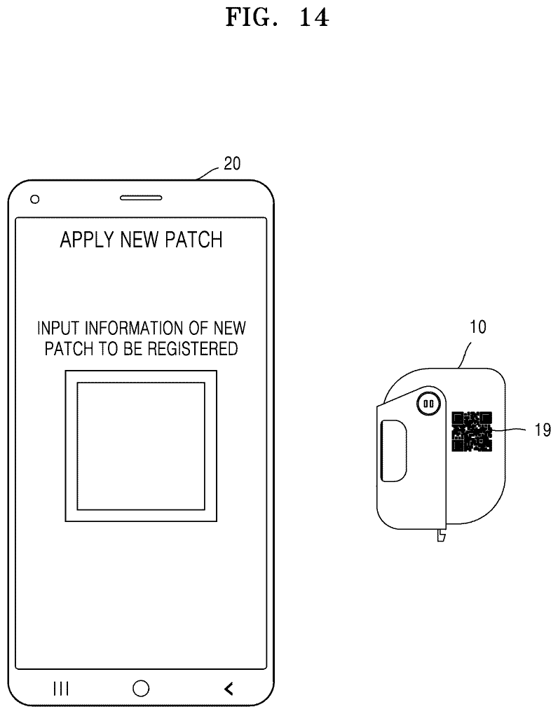

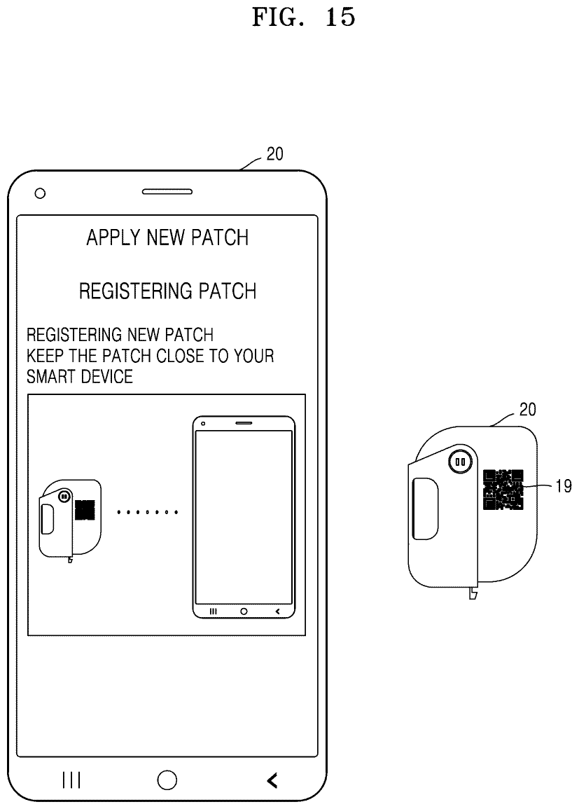

FIG. 12 is a block diagram illustrating a configuration of the medical liquid infusion device 10 and the controller 20, according to an embodiment of the disclosure. FIG. 13 is a flowchart illustrating a method of wireless communication connection with the medical liquid infusion device 10, according to an embodiment of the disclosure. FIGS. 14 and 15 are explanatory diagrams illustrating some operations of a method of wireless communication connection with the medical liquid infusion device 10. Hereinafter, a method of wireless communication connection for pairing a target medical liquid infusion device 10 that a user desires, with the controller 20, even when a plurality of medical liquid infusion devices 10 and 10a exist around the controller 20, will be described with reference to FIGS. 12 to 15.

Referring to FIG. 13, in operation 101, the target medical liquid infusion device 10, on an outer surface of which an information code including an identifier is marked, is prepared. Hereinafter, a medical liquid infusion device that a user desires to connect with the controller 20 is referred to as a target medical liquid infusion device 10, and a medical liquid infusion device that the user does not desire to connect with the controller 20 is referred to as a non-target medical liquid infusion device 10a. Here, the information code, the identifier, a position where the information code is marked, and the like have been described above with reference to FIGS. 11A and 11B, and thus, redundant descriptions thereof will be omitted.

In operation 102, the controller 20 reads the information code of the target medical liquid infusion device 10. Referring to FIG. 12, the controller 20 may include an information code reader 201, and operation 102 may be performed by using the information code reader 201. Here, the information code reader 201 may be implemented in various forms such as a camera, a QR code reader, or a data matrix code reader. For example, referring to FIG. 14, the controller 20 may capture an image of the information code 19 marked on the outer surface of the medical liquid infusion device 10 by using a built-in camera, and read content from the information code in the captured image by using a code reading algorithm of the information code reader 201.

In operation 103, the controller 20 recognizes and stores the identifier within the read information code. Referring to FIG. 12, the controller 20 may include an information recognition unit 202, and operation 103 may be performed by the information recognition unit 202. The information recognition unit 202 may store an identification algorithm, and recognizes, extracts, and stores the identifier from the content of the read information code by using the identification algorithm. Here, the identifier includes unique identification information of the target medical liquid infusion device 10 on which the information code is marked, and may include, for example, at least one of a unique name, a serial number, and a unique wireless communication address of the medical liquid infusion device 10.

In operation 104, a medical liquid is injected into the target medical liquid infusion device 10. The user injects the medical liquid into a medical liquid injection port marked on the outer surface of the target medical liquid infusion device 10, by using a syringe.

In operation 105, the target medical liquid infusion device 10, triggered by the medical liquid injection, transmits a pairing request signal including the identifier to its surroundings. When the medical liquid is injected, the controller 20 displays a screen such as that illustrated in FIG. 15, and the process from transmitting the pairing request signal to completing wireless communication connection between the two devices may be performed automatically without requiring separate user intervention.

Here, the pairing request signal is a type of signal transmitted by a first device to perform advertising for performing wireless communication connection with a nearby second device. In general, a pairing request signal does not include much information about the first device and but includes only minimal information. However, according to an embodiment of the disclosure, to perform wireless communication connection between the target medical liquid infusion device 10 and the controller 20, the pairing request signal includes the identifier that is included in the information code 19. Referring to FIG. 12, the medical liquid infusion device 10 may include a pairing request signal generation unit 101 and a wireless communication interface 102, and operation 105 may be performed by the pairing request signal generation unit 101 generating a pairing request signal including the identifier, and the wireless communication interface 102 may advertise the generated pairing request signal.

Meanwhile, it may be assumed that, in addition to the target medical liquid infusion device 10 that the user wants to pair, the non-target medical liquid infusion device 10a that the user does not want to pair is located around the controller 20. Accordingly, in operation 105a, the non-target medical liquid infusion device 10a may also generate a pairing request signal including an identifier a.

In operation 106, the controller 20 scans pairing request signals from nearby medical liquid infusion devices. Scanning by the controller 20 may start from when the information code 19 is recognized, but is not limited thereto, and may start from when the user requests scanning. The scanning operation may be performed by a wireless communication connection unit 204 within the controller 20 illustrated in FIG. 12.

In operation 107, when the controller 20 receives one pairing request signal during scanning, the controller 20 determines whether an identifier included in the received pairing request signal matches the identifier within the previously recognized information code. Referring to FIG. 12, the controller 20 may include an identifier match determination unit 203, and operation 107 may be performed by this identifier match determination unit 203. The identifier match determination unit 203 extracts the identifier from the pairing request signal and then, by using a comparison algorithm that compares the extracted identifier with the stored identifier, determines whether the two identifiers are identical to each other.

For example, when a pairing request signal from the non-target medical liquid infusion device 10a is received by the controller 20 with a slightly higher strength, the controller 20 extracts the identifier a included in the pairing request signal from the non-target medical liquid infusion device 10a and then compares the extracted identifier with the stored identifier. In this case, because the two identifiers do not match, from this point on, the controller 20 ignores pairing request signals transmitted from the non-target medical liquid infusion device 10a and resumes (restarts) scanning for pairing request signals.

For example, when the pairing request signal from the target medical liquid infusion device 10 is received by the controller 20 with a slightly higher strength, or when the pairing request signal from the target medical liquid infusion device 10 is received while pairing request signals from the non-target medical liquid infusion device 10a are being ignored, the controller 20 extracts the identifier included in the pairing request signal from the target medical liquid infusion device 10 and then compares the extracted identifier with the stored identifier. In this case, because the two identifiers match, as in operation 108 of FIG. 13, the controller 20 requests a wireless communication connection from the target medical liquid infusion device 10. In this way, the controller 20 repeats the scanning process until it finds a medical liquid infusion device whose identifier matches the stored identifier.

In operation 109, the target medical liquid infusion device 10 responds to the wireless communication connection request from the controller 20. In operation 110, the wireless communication connection starts, and the two devices exchange connection parameters. In operation 111, the wireless communication connection between the two devices is completed. Thereafter, the two devices may freely transmit and receive data through wireless communication. Operations 109 to 111 are performed by operations of the wireless communication interface 102 (see FIG. 12) of the medical liquid infusion device and the wireless communication connection unit 204 (see FIG. 12) of the controller 20. Details related to the wireless communication connection in operations 109 to 111 are similar to a method adopted in general Bluetooth or Bluetooth Low Energy and are well known to a person skilled in the art, and thus, detailed descriptions thereof will be omitted.

In operation 112, when the wireless communication is established, the target medical liquid infusion device 10 confirms the pairing through a buzzer sound. In operation 113, a user attaches the target medical liquid infusion device 10 paired with the controller 20 to their body and inserts a cannula into their body, so as to prepare for a medical liquid to be infused. When, in operation 114, an infusion signal is generated by the controller 20 and then transmitted to the target medical liquid infusion device 10 with which wireless communication is established, in operation 115, according to the transmitted infusion signal, a medical liquid infusion control unit 103 (see FIG. 12) of the medical liquid infusion device 10 controls a pump to infuse the medical liquid into the user's body.

According to an embodiment of the disclosure, because the target medical liquid infusion device 10 that the user desires may be paired with the controller 20 by utilizing an information code even when a plurality of medical liquid infusion devices exist around the controller 20, user convenience is increased, and problems that occur when a non-target medical liquid infusion device 10a is paired with the controller 20 contrary to the user's intention may be blocked.

FIG. 16 is a block diagram illustrating a configuration of the medical liquid infusion device 10 and the controller 20, according to another embodiment of the disclosure. FIG. 9 is a flowchart illustrating a method of wireless communication connection with the medical liquid infusion device 10, according to another embodiment of the disclosure.

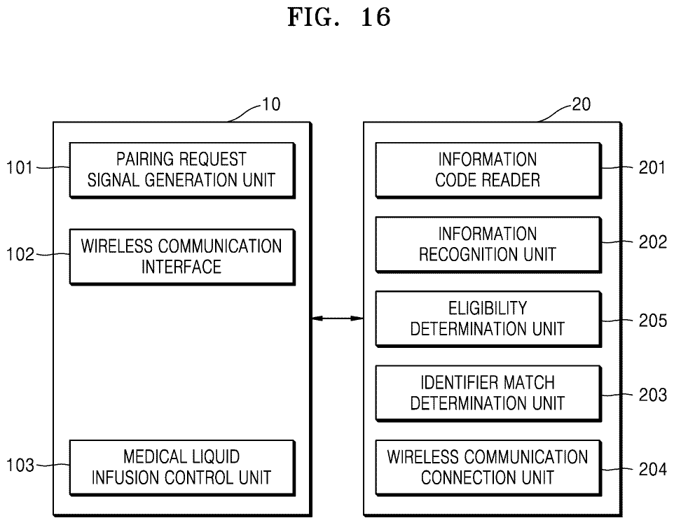

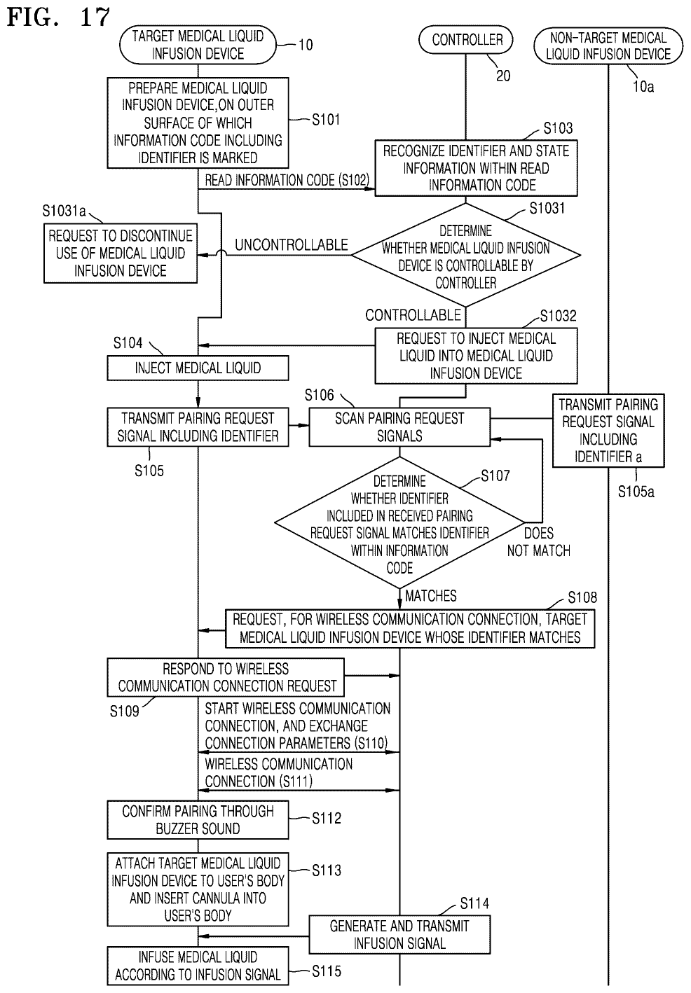

The embodiment illustrated in FIGS. 16 and 17 discloses a method of wireless communication connection with the medical liquid infusion device 10, wherein, before a medical liquid is injected into the medical liquid infusion device 10 to generate a pairing request signal for pairing, the controller 20 determines a state of the target medical liquid infusion device 10 by using the information code 19, so as to prevent pairing with a medical liquid infusion device 10 that is in an ineligible state. Thus, the embodiment illustrated in FIGS. 16 and 17 is the same as the embodiment described above with reference to FIGS. 12 to 16, except that operations are added in which the controller 20 determines a state of the target medical liquid infusion device 10 by using the information code 19, and thus, redundant descriptions of the operations will be omitted.

Referring to FIG. 17, in operation 101, the medical liquid infusion device 10, on an outer surface of which an information code 19 including state information in addition to an identifier is marked, is prepared. Here, the state information may include conditions for determining whether the medical liquid infusion device 10 is controllable by the controller 20, whether the medical liquid infusion device 10 is able to interwork with the controller 20, or whether the medical liquid infusion device 10 is capable of wireless communication connection with the controller 20. For example, the state information may include information about a model, a version, or a regional restriction, and the like, of the medical liquid infusion device 10. Other aspects of the information code, the identifier, the position where the information code is marked, and the like have already been described above, and thus, redundant descriptions thereof will be omitted.

In operation 102, the controller 20 reads the information code of the target medical liquid infusion device 10.

In operation 103, the controller 20, in addition to recognizing and storing the identifier within the read information code, also recognizes and stores the state information. Referring to FIG. 16, the controller 20 may include the information recognition unit 202, and operation 103 may be performed by the information recognition unit 202. The information recognition unit 202 may store an identification algorithm, and recognizes, extracts, and stores the identifier and the state information from the content of the read information code by using the identification algorithm.

In operation 1031, the controller 20 determines, based on the stored state information, whether the target medical liquid infusion device 10 is controllable by the controller 20. That is, the controller 20 determines an eligibility status of the target medical liquid infusion device 10. Here, the eligibility status refers to determining whether the medical liquid infusion device 10 is a model or version controllable by the controller 20, or is subject to a regional restriction setting, whether the medical liquid infusion device 10 is a model or version that may interwork with the controller 20, or is subject to a regional restriction setting, or whether the medical liquid infusion device 10 is a model or version capable of wireless communication connection with the controller 20, or is subject to a regional restriction setting. Referring to FIG. 16, the controller 20 may include an eligibility determination unit 205, and operation 1031 may be performed by the eligibility determination unit 205. In detail, the eligibility determination unit 205 receives, from the integrated management server 30 (see FIG. 9), and stores eligibility information about models, versions, or regional restrictions, and the like, pertaining to medical liquid infusion devices 10 that are controllable by the corresponding controller 20 through data transmission and reception via a wireless communication connection. The eligibility determination unit 205 compares the stored eligibility information with the state information read from the information code, to determine the eligibility status of the target medical liquid infusion device 10.

For example, in a case in which the controller 20 is able to establish a wireless communication connection with only a medical liquid infusion device of a specific version or higher, but the target medical liquid infusion device 10 whose information code has been recognized is below that specific version, or in a case in which the controller 20 is able to establish a wireless communication connection with only a medical liquid infusion device produced in the corresponding country, but the target medical liquid infusion device 10 whose information code has been recognized is a product from another country, the eligibility determination unit 205 makes a determination of ineligibility. As such, when control of the target medical liquid infusion device by the controller 20 is impossible, the controller 20 requests the user to discontinue use of the medical liquid infusion device 10. By generating a warning sound or light through an output unit (not shown) or by displaying a warning pop-up, the controller 20 requests the user not to inject a medical liquid into the target medical liquid infusion device 10 whose information code has been recognized. Through this, waste of the medical liquid is prevented, and unnecessary use of the medical liquid infusion device is avoided (operation 1031a).

On the other hand, in a case in which the medical liquid infusion device 10 is a model and version controllable by the controller 20 and has no regional restrictions, the eligibility determination unit 205 makes a determination of eligibility. As such, when control of the target medical liquid infusion device by the controller 20 is possible, the controller 20 requests the injection of a medical liquid into the target medical liquid infusion device 10, such that a wireless communication connection may be established between the target medical liquid infusion device 10 and the controller 20. By displaying a medical liquid injection request pop-up through an output unit, the controller 20 may request the user to inject a medical liquid into the target medical liquid infusion device 10 whose information code has been recognized (operation 1032).

In operation 104, a medical liquid is injected into the target medical liquid infusion device 10. The user injects the medical liquid into a medical liquid injection port marked on the outer surface of the medical liquid infusion device, by using a syringe.

In operation 105, the target medical liquid infusion device 10, triggered by the medical liquid injection, transmits a pairing request signal including the identifier to its surroundings.

In operation 106, the controller 20 scans pairing request signals from nearby medical liquid infusion devices.

In operation 107, when the controller 20 receives one pairing request signal during scanning, the controller 20 determines whether an identifier included in the received pairing request signal matches the identifier within the previously recognized information code.

When the received pairing request was transmitted from the non-target medical liquid infusion device 10a and thus the two identifiers do not match, the controller 20 ignores pairing request signals transmitted from the non-target medical liquid infusion device 10a and resumes scanning for pairing request signals.

When the received pairing request was transmitted from the target medical liquid infusion device 10 and thus the two identifiers match, the controller 20 requests a wireless communication connection from the target medical liquid infusion device 10, as in operation 108.

In operation 109, the target medical liquid infusion device 10 responds to the wireless communication connection request from the controller 20.

In operation 110, the wireless communication connection starts, and the two devices exchange connection parameters. In operation 111, the wireless communication connection is completed.

Operations 105 to 115 are the same as those of FIG. 13, and thus, redundant descriptions thereof will be omitted.

According to another embodiment of the disclosure, an effect of preventing waste of a medical liquid and unnecessary disposal of a medical liquid infusion device is provided as, before a medical liquid is injected into the target medical liquid infusion device 10, the controller 20 determines, by using the information code read from the target medical liquid infusion device 10, whether the target medical liquid infusion device 10 is suitable for connection to the controller, thereby preventing pairing with a medical liquid infusion device that is in an ineligible state.

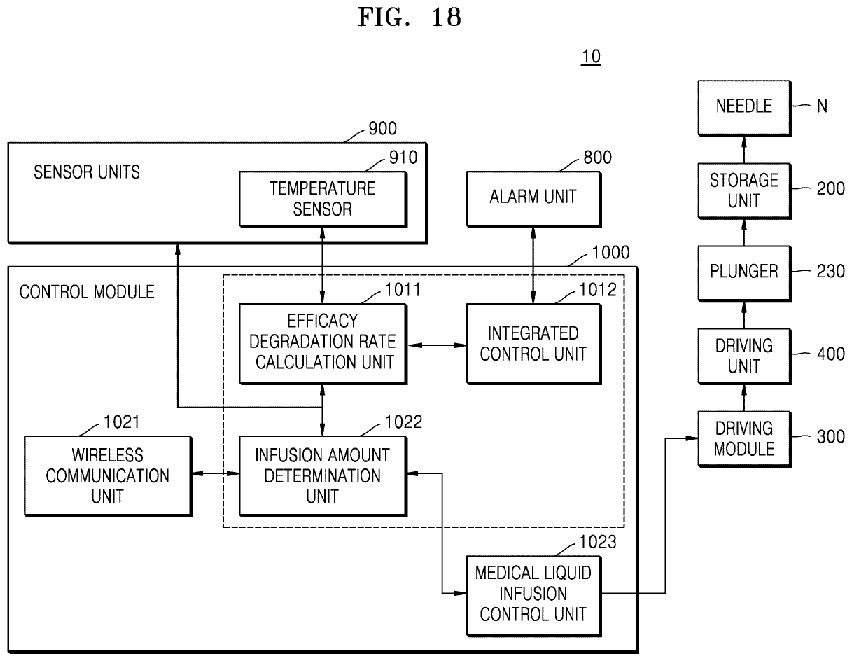

The disclosure discloses various embodiments of a method of determining a medical liquid infusion amount in a medical liquid infusion device. Hereinafter, descriptions will be provided with reference to FIGS. 18 to 26.

FIG. 18 is a block diagram schematically illustrating a control module 1000 of a medical liquid infusion device, and constituent components associated with the control module 1000, according to another embodiment of the disclosure.

The medical liquid infusion device 10 may include a needle N, a storage unit 200, a plunger 230, a driving module 300, a driving unit 400, an alarm unit 800, a plurality of sensor units 900 including a temperature sensor 900, and the control module 1000. For the needle N, the storage unit 200 (201 of FIG. 4), the plunger 230, the driving module 300, and the driving unit 400, reference is made to the description provided above with reference to FIG. 4.

The alarm unit 800 may generate an alarm by sound, light, vibration, or the like, in response to a signal from the control module 1000.

The sensor units 900 may measure a number of rotations or a rotational speed of the driving unit 400, an amount of medical liquid stored in the storage unit 200, whether an injection port is occluded, whether there is medical liquid leakage, a device abnormality, a remaining battery capacity, and the like, and transmit an electrical signal corresponding to the measurements to the control module 1000. The sensor units 900 may also transmit, to the control module 1000, an electrical signal corresponding to a measured biometric signal of a user.

The sensor units 900 may include a temperature sensor 910, and the temperature sensor 910 may be arranged in the storage unit 200. In detail, the temperature sensor 210 may measure a temperature of a medical liquid stored in the storage unit 200, and transmit an electrical signal corresponding to the measured temperature to the control module 1000. The temperature sensor 910 may be arranged on an upper surface, a lower surface, a side surface, or the like, of the storage unit 200. The temperature sensor 910 may be arranged inside or outside the storage unit 200. The temperature sensor 910 may be, for example, a thermocouple, a metal resistance temperature detector, a thermistor, an integrated circuit (IC) temperature sensor, or a magnetic temperature sensor, or the like. According to an embodiment, the temperature sensor 910 may be arranged on an outer upper surface of the storage unit 200 that faces the housing 11, and accordingly, an influence from a user's body temperature may be excluded.

The control module 1000 may include a wireless communication unit 1021, an efficacy degradation rate calculation unit 1011, an infusion amount determination unit 1022, an integrated control unit 1012, and a medical liquid infusion control unit 1023. FIG. 18 illustrates only constituent components associated with an embodiment of the disclosure, but the control module 1000 may further include various constituent components necessary for an operation of the medical liquid infusion device 10, in addition to the illustrated constituent components. Each constituent component included in the control module 1000 may be operated by a processing device such as a processor.

The wireless communication unit 1021 establishes a wireless communication connection enabling the controller 20 and the medical liquid infusion device 10 to transmit and receive various signals and data. The wireless communication unit 1021, upon a medical liquid being injected into the storage unit 200, i.e., triggered by the medical liquid injection, performs advertising to transmit an advertising message, receives a connection request signal from the controller 20, and completes the wireless communication connection by transmitting and receiving connection parameters once the wireless connection starts. For example, the wireless communication unit 1021 may be a Bluetooth or Bluetooth Low Energy module.

The integrated control unit 1012 controls each constituent component of the medical liquid infusion device 10 based on various signals. For example, the integrated control unit 1012 may control the on/off state and the like of the alarm unit 800 by using a signal that controls the alarm unit 800, and may receive, from the sensor units 900, signals corresponding to measurements of a number of rotations or a rotational speed of the driving unit, an amount of medical liquid stored in the storage unit, whether the injection port is occluded, whether there is medical liquid leakage, a device abnormality, a remaining battery capacity, and the like, and also receive signals corresponding to measurements of a user's blood glucose level, blood pressure, heart rate, and the like. The integrated control unit 1012 may generate device data and biometric values based on these signals.

Based on an infusion signal from the control module 1000 or an infusion signal transmitted from the controller 20, the medical liquid infusion control unit 1023 controls the driving module 300 such that the medical liquid stored in the storage unit 200 is discharged through the needle N.

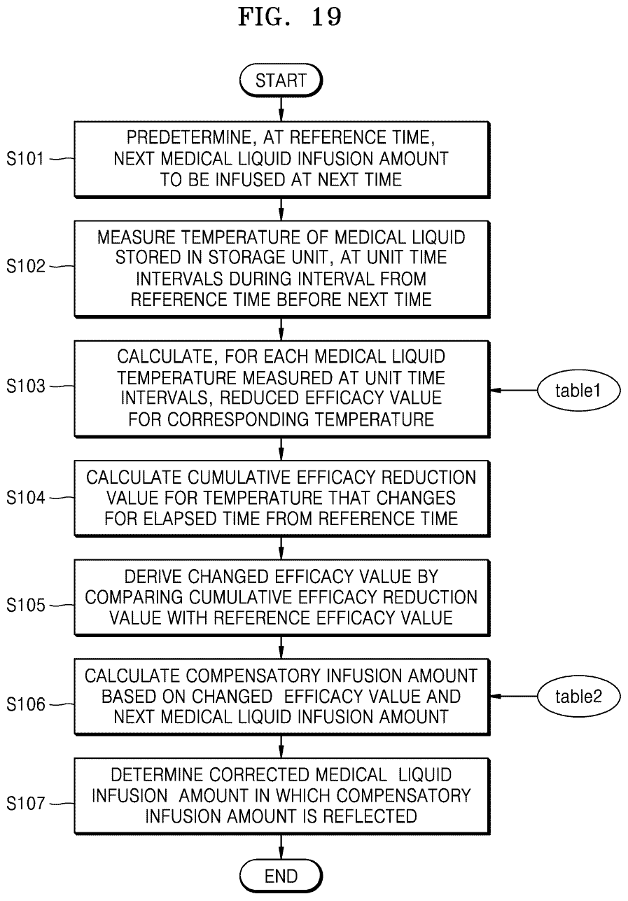

The control module 1000 according to an embodiment of the disclosure determines a medical liquid infusion amount, reflecting a situation where the medical liquid contained in the storage unit 200 ages according to an external environment and its efficacy degrades. Furthermore, according to another embodiment of the disclosure, the control module 1000 determines a medical liquid infusion amount, reflecting a situation where the efficacy changes according to an attachment site of the medical liquid infusion device 10 or a state of the user. Through this, the medical liquid infusion device 10 according to an embodiment of the disclosure has an effect of ideally realizing the effect of a medical liquid on a subject, by being tailored to the actual state of the medical liquid and the state of the user. In connection with this embodiment, the control module 1000 may include the efficacy degradation rate calculation unit 1011 and the infusion amount determination unit 1022. Hereinafter, the efficacy degradation rate calculation unit 1011 and the infusion amount determination unit 1022 will be described with reference to FIG. 19.

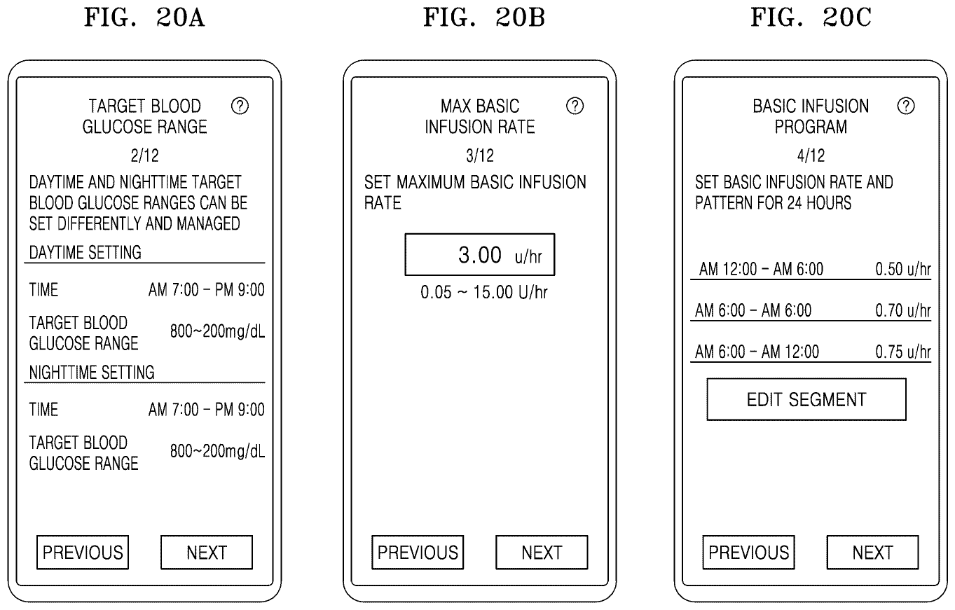

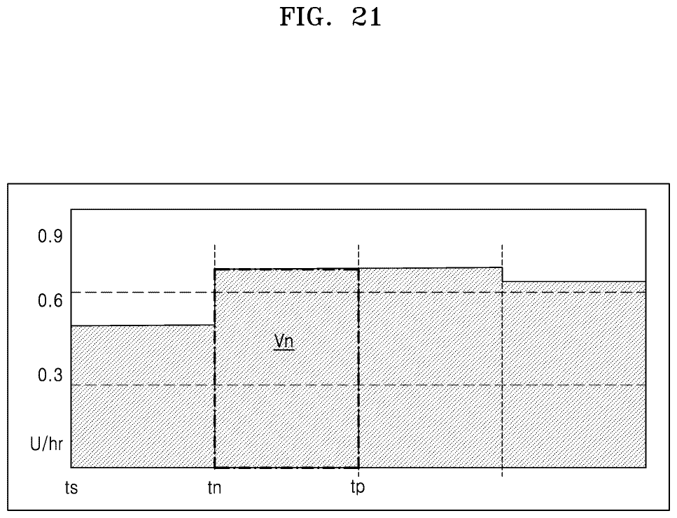

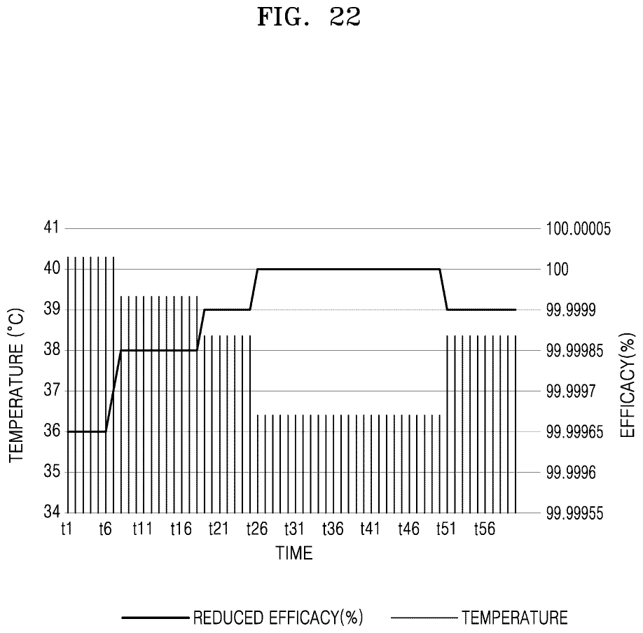

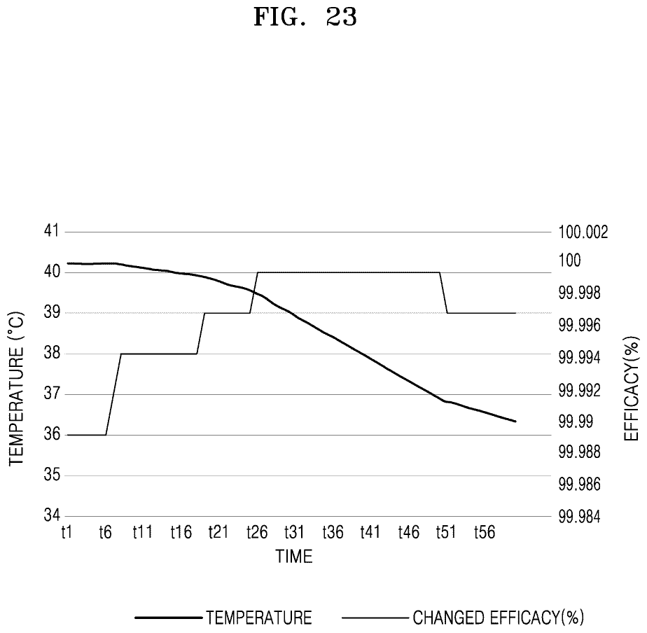

FIG. 19 is a flowchart illustrating a method of determining a medical liquid infusion amount, according to an embodiment of the disclosure. FIGS. 20A to 20C illustrate input/output screens of a controller for explaining a medical liquid infusion method according to a basic infusion program. FIG. 21 is a graph illustrating a medical liquid infusion amount by time according to a basic infusion program. FIG. 22 is a graph illustrating a change in efficacy per hour according to temperature. FIG. 23 is a graph illustrating a cumulative change in efficacy according to temperature and time. FIG. 24 is a graph illustrating that the medical liquid infusion amount by time according to the basic infusion program of FIG. 21 has been compensated.

In operation 101 of FIG. 19, at a reference time ts, a next medical liquid infusion amount Vn to be infused at a next time tn is predetermined by the control module 1000. Here, the reference time ts is a current or past time point when an infusion signal was last input to the medical liquid infusion control unit 1023, and the medical liquid was consequently infused. Here, the next time tn is a future time point when medical liquid is to be infused as a predetermined next infusion signal is input to the medical liquid infusion control unit 1023. Here, the next medical liquid infusion amount Vn may be predetermined by the basic infusion program.

Referring to FIGS. 20 and 21, the basic infusion program is an infusion method that schedules in advance for a user-set amount of medical liquid to be infused at a user-set rate at a user-specified time during a preset period (e.g., 24 hours). The basic infusion program may be set by using the controller 20 that interworks with the medical liquid infusion device 10, or by using a platform provided by the integrated management server 30, such as a web or an application. Referring to FIG. 20A, the user may set a target blood glucose range through an input/output module of the controller 20. That is, daytime and nighttime periods may be defined, and then a target blood glucose range to be achieved in each time period may be set. Because blood glucose levels are likely to remain high during the day due to food consumption, a target blood glucose range different from that for the nighttime may be set to lower blood glucose accordingly and maintain it within a certain range. Referring to FIG. 20B, the user may set a maximum basic infusion rate of a medical liquid, and referring to FIG. 20C, the user may set different basic infusion rates for different time periods. Because blood glucose may rise rapidly after food consumption, setting different basic infusion rates for different time periods in conjunction with meal or snack times helps to keep the blood glucose level within a normal blood glucose range.

Referring to FIG. 21, according to the basic infusion program set in FIG. 6, the medical liquid is infused in amounts and at rates (U/hr) that are set for the respective time periods. For example, a period from ts to tn may be a nighttime or dawn time period, a period from tn to tp may be a morning to lunchtime period, and a period after tp may be an evening to nighttime period. In this case, it may be confirmed that a relatively small amount of medical liquid is set to be infused during the night or before morning, and that a relatively large amount of medical liquid is set to be infused from after morning until evening. Here, U on the vertical axis of FIG. 7 denotes a unit, where 1 unit is 1/24 mg of purified insulin, and 1 mg of insulin signifies 24 units. The horizontal axis represents time (hour). Thus, the area of the shaded portion in the graph corresponds to the amount of medical liquid administered or to be administered during the corresponding time, that is, the amount of insulin (U). Therefore, the area of the portion indicated as Vn corresponds to the next medical liquid infusion amount Vn.

Referring again to operation 102 of FIG. 19, the control module 1000 receives, from the temperature sensor 910, a signal at regular unit time intervals t during an interval from the reference time ts to the next time tn, wherein the signal represents a measured temperature of the medical liquid stored in the storage unit 200. Here, the unit time interval t may be an interval determined by the user and may be determined to be, for example, 1 second (sec), 30 seconds, 1 minute (min), 5 minutes, 10 minutes, or the like.

FIG. 22 illustrates that a time interval between the reference time ts and the next time tn is 60 minutes, and includes a solid line graph representing the temperature of the medical liquid stored in the storage unit 200, measured at unit time intervals t. Here, the unit time interval t was set to 1 minute, and the interval between the reference time ts and the next time tn, and the duration of the unit time interval t are not limited to those illustrated and may vary. FIG. 22 illustrates, as a solid line graph, results of measuring the temperature of the medical liquid a total of 60 times (once per minute) to determine the amount of medical liquid to be infused 1 hour after the reference time ts. Referring to the solid line graph, it may be confirmed that the temperature of the medical liquid was measured as 36 degrees Celsius during the period from 1 minute to 6 minutes, 38 degrees Celsius during the period from 7 minutes to 18 minutes, 39 degrees Celsius during the period from 19 minutes to 25 minutes, 40 degrees Celsius during the period from 26 minutes to 50 minutes, and 39 degrees Celsius during the period from 51 minutes to 60 minutes.

When a medical liquid is exposed to high temperatures for a certain period or longer, its efficacy is reduced. According to animal experiments, it has been reported that, compared to the efficacy of insulin at 26 degrees Celsius, when a temperature of 37 degrees Celsius persists for 1 hour, the efficacy of insulin degrades by about 14 to 18 percent. The medical liquid infusion device 10 is a device to be attached to the exterior of a subject's body to automatically infuses a medical liquid to control the subject's blood glucose level, and unlike conventional insulin pumps, it has no tube, is small, lightweight, features a waterproof design, and may be used for 1 day or longer, thereby enabling a user to perform various daily and leisure activities while the medical liquid infusion device 10 is attached. However, when the user is exposed to high temperatures for a certain period or longer, such as when taking a shower or spa with hot water, sleeping or living on an Ondol (Korean underfloor heating system) or a heating mat, or performing outdoor activities in hot weather, the efficacy of the medical liquid stored in the medical liquid infusion device 10 is reduced. When such a situation, where the medical liquid contained in the storage unit 200 ages according to an external environment and its efficacy degrades, is not reflected in a medical liquid infusion amount, a problem occurs in that the user's blood glucose level cannot reach the target blood glucose range. An embodiment of the disclosure may solve such a problem and, by compensating a medical liquid infusion amount to be tailored to the actual state of the medical liquid, ideally realize the effect of the medical liquid on a subject.

Referring again to operation 103 of FIG. 19, the control module 1000, for each medical liquid temperature measured at unit time intervals t, calculates an efficacy reduction value Rtempₜ for the corresponding temperature during the unit time interval t. Operation 103 may be performed by the efficacy degradation rate calculation unit 1011 (see FIG. 18) of the control module. The efficacy degradation rate calculation unit 1011 may store a table table1 that includes, for each type of medical liquid and for each temperature, the degree to which efficacy is reduced upon exposure at the corresponding temperature for a unit time interval. For example, Table 1 below illustrates a portion of such a table for insulin. Here, the unit time interval t is 1 minute, and the temperatures are 38 degrees Celsius, 39 degrees Celsius, and 40 degrees Celsius. Table 1 exemplarily shows a processed portion of the table for explanatory purposes, and actual efficacy degradation values according to temperature may differ from those in Table 1.

TABLE 1

| | | Degree of efficacy |
|---|---|---|
| Medical liquid type | Temperature (° C.) | degradation |
| Insulin | 40 | 1/(360000X2⁰) |
| | 39 | 1/(360000X2¹) |
| | 38 | 1/(360000X2²) |

The efficacy degradation rate calculation unit 1011 calculates an efficacy reduction value for the corresponding temperature during a unit time interval, by using the stored table. For example, Table 2 below shows results of calculating efficacy reduction values by using Table 1, assuming that the efficacy value at a reference temperature is 1.

TABLE 2

| | | Reduced efficacy value |
|---|---|---|
| Medical liquid type | Temperature (° C.) | Rtempₜ |
| Insulin | 40 | 1 − 1/(360000X2⁰) = |
| | | 0.999997222 |
| | 39 | 1 − 1/(360000X2¹) = |
| | | 0.999998611 |
| | 38 | 1 − 1/(360000X2²) = |
| | | 0.9999993056 |

FIG. 22 includes a column graph illustrating a reduced efficacy value for each unit time interval t, based on the calculation results of Table 2. By comparing the solid line graph with the column graph, it may be confirmed that the efficacy tends to decrease as the temperature increases within a specific temperature range. Referring again to operation 104 of FIG. 19, the control module calculates a cumulative efficacy reduction value for the temperature that changes for an elapsed time r from the reference time ts to the next time tn. Operation 104 may also be performed by the efficacy degradation rate calculation unit 1011 of the control module. The efficacy degradation rate calculation unit 1011 may derive the cumulative efficacy reduction value by accumulating and summing the results of calculating the efficacy reduction values for respective unit time intervals t within the elapsed time r. Thus, the more the medical liquid is exposed to high temperatures and the longer the exposure time at such high temperatures, the larger the cumulative efficacy reduction value becomes.

Referring to operation 105, a changed efficacy value D, which indicates the value to which the efficacy has decreased, is derived by comparing the calculated cumulative efficacy reduction value with a reference efficacy value I. Operation 105 may also be performed by the efficacy degradation rate calculation unit 1011. The efficacy degradation rate calculation unit 1011 may derive the changed efficacy value D by using Equation 1. Referring to Equation 1, t denotes a unit time interval and may be determined as 1 second (sec), 30 seconds, 1 minute (min), 5 minutes, 10 minutes, or the like, according to a user's setting, however, herein it was set to 1 minute. r denotes an elapsed time from a reference time, and herein it may be a time within a range of more than 1 minute (min) and less than 60 minutes. That is, the changed efficacy value D may be calculated for each unit time intervals t until the next time tn, and then stored in the efficacy degradation rate calculation unit 1011, and the stored value may be updated at unit time intervals. tempₜ denotes a temperature value for a unit time interval t, R denotes an efficacy reduction value, and thus, Rtempₜ represents the efficacy reduction value Rtempₜ for a specific medical liquid at the corresponding temperature during the unit time period, and herein, it represents the efficacy reduction value Rtempₜ for insulin at the corresponding temperature during 1 minute. Herein, an initial value of 1 was used as the reference efficacy value I. The changed efficacy value D may be expressed as a real number within a range of greater than 0 and less than or equal to 1. (0<D≤1)

$$D = 1 - {\sum }_{t = 0}^{r} {Rtemp}_{t} \qquad [ Equation 1 ]$$

FIG. 23 includes a first solid line graph illustrating a changed efficacy value D calculated by using Equation 1. In FIG. 23, the efficacy value is expressed as a percentage (%) for ease of understanding. By comparing a second solid line graph illustrating temperature with the first solid line graph illustrating the changed efficacy value D, it may be confirmed that the changed efficacy value D tends to decrease continuously due to changes in temperature. That is, as the medical liquid is exposed to high temperatures, it may be confirmed that the efficacy tends to decrease gradually compared to the reference efficacy.

Referring to FIG. 23, it may be confirmed that, between the reference time ts and the next time tn, the medical liquid was exposed at 38 degrees Celsius for a total of 11 minutes, at 39 degrees Celsius for a total of 17 minutes, and at 40 degrees Celsius for a total of 25 minutes. Thus, when the reference efficacy value I is considered as 1, the changed efficacy value D over a total of 60 minutes from the reference time ts to the next time tn is calculated as 0.99989930. When the reference efficacy value I at the reference time ts is 100%, it may be confirmed that, as the medical liquid is exposed to high temperatures, the efficacy value decreases to 99.98% at the next time tn. Although FIG. 23 considers only the changed efficacy value for a 60-minute period for ease of understanding, when actually using the basic infusion program, the time interval between the reference time ts and the next time tn may be 4 hours to 8 hours. Therefore, when the medical liquid is exposed to high temperatures, the efficacy value may decrease by about 1% to 10%. Because such a decrease in the efficacy value may cause problems in reaching a target blood glucose level, a compensatory infusion of the medical liquid is necessary.

Referring to operation 106, the control module 1000 calculates a compensatory infusion amount E based on the derived changed efficacy value D and the previously set next medical liquid infusion amount Vn. Operation 106 may be performed by the infusion amount determination unit 1022 (see FIG. 18) of the control module 1000.

According to an embodiment of the disclosure, the infusion amount determination unit 1022 may derive the compensatory infusion amount E by using the next medical liquid infusion amount Vn and the changed efficacy value D as factors. In detail, the changed efficacy value D is a value indicating the value to which the efficacy has changed compared to the complete efficacy (1 or 100%). Thus, when the changed efficacy value D is 0.8735 or 87.35%, it means that the efficacy has decreased by 0.1265 or 12.65% compared to the complete efficacy. Therefore, to achieve the complete efficacy of 1 or 100%, it is necessary to additionally infuse a specific amount of medical liquid. The compensatory infusion amount E is an amount of medical liquid additionally infused to achieve the complete efficacy of 1 or 100%. A compensatory infusion amount proportion e represents an amount of medical liquid that needs to be compensatorily infused, based on the complete efficacy of 1 or 100%.

Here, the complete efficacy of 1 may be expressed by the following equation by using the changed efficacy value D. Here, D denotes the changed efficacy value and is not 0.

$$1 = 1 \times D + \frac{( 1 - D )}{D} \qquad [ Equation 2 ]$$

The compensatory infusion amount proportion e is determined by the following equation. Here, D denotes the changed efficacy value and is not 0.

$$e = \frac{( 1 - D )}{D} \qquad [ Equation 3 ]$$

Referring to the equation, when the changed efficacy value D is 0.8735, the compensatory infusion amount proportion e is calculated as approximately 0.1448. Thus, the compensatory infusion amount E is a value resulting from multiplying the next medical liquid infusion amount Vn by the compensatory infusion amount proportion e. In other words, the compensatory infusion amount E may be a value resulting from multiplying the next medical liquid infusion amount Vn by a value obtained by dividing the difference between the complete efficacy value and the changed efficacy value by the changed efficacy value. For example, for insulin, when the changed efficacy value D has been 0.8735 due to exposure to high temperatures for a certain time period, the compensatory infusion amount proportion e for applying 100% efficacy is approximately 0.1448 when referring to Equation 3. Thus, when the next medical liquid infusion amount Vn is determined as 100 U, 14.48 U of medical liquid, as the compensatory infusion amount E, needs to be additionally infused. However, the method of determining the compensatory infusion amount is not limited thereto, and the infusion amount determination unit 1022 may separately store a table table2 for deriving the compensatory infusion amount E based on the ratio of the next medical liquid infusion amount Vn and the changed efficacy value D, and determine the compensatory infusion amount E by extracting a matched value from the table according to the changed efficacy value D.

In operation 107, the control module 1000 derives a corrected medical liquid infusion amount V′n in which the compensatory infusion amount E is reflected. Referring to FIG. 24, it may be confirmed that the compensatory infusion amount E has been added to the graph of FIG. 21. In FIG. 24, U on the vertical axis denotes a unit, and the horizontal axis represents time (hour). Thus, the area of the shaded portion in the graph corresponds to the amount of medical liquid administered or to be administered during the corresponding time, that is, the amount of insulin (U). It may be confirmed that the next medical liquid infusion amount Vn to be administered from the next time tn until before a next time tp has been changed to the corrected medical liquid infusion amount V′n by the addition of the compensatory infusion amount E to compensate for the efficacy reduction due to high temperature exposure.

According to an embodiment of the disclosure, provided are a medical liquid infusion device and method that enable smart control considering not only simply infusing a preset amount of medical liquid but also changes in efficacy due to a surrounding environment, by calculating the degree to which the efficacy of a medical liquid decreases when exposed to high temperatures during the use of the medical liquid infusion device, and reflecting the calculated degree in the next medical liquid infusion amount.

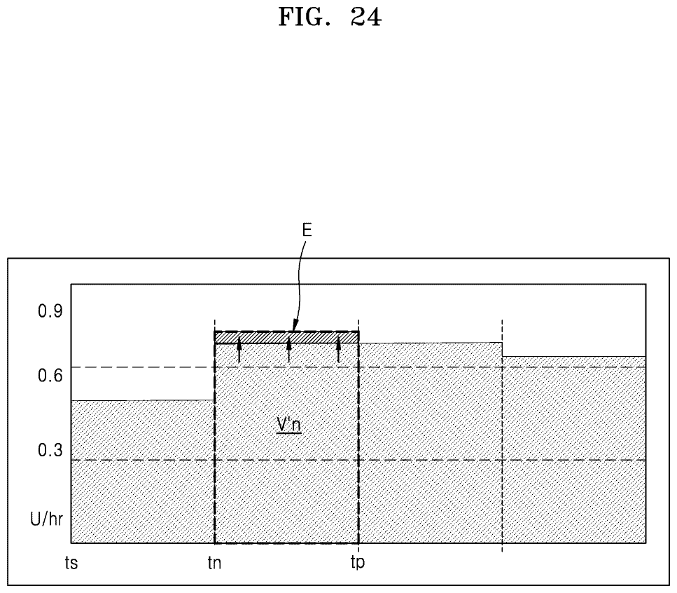

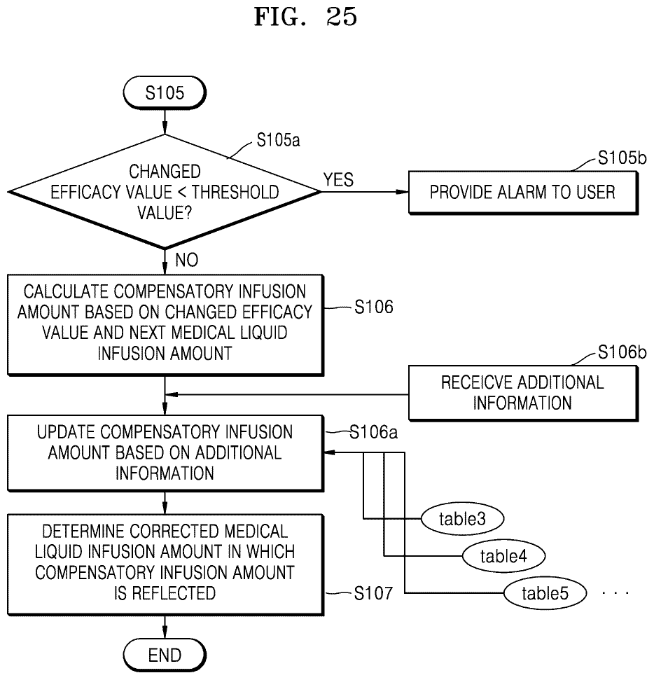

FIG. 25 is a flowchart illustrating a method of determining a medical liquid infusion amount, according to another embodiment of the disclosure.

According to another embodiment of the disclosure, the infusion amount determination unit 1022 may update the compensatory infusion amount E by using, as factors, additional information in addition to the next medical liquid infusion amount Vn and the changed efficacy value D. As described above, the infusion amount determination unit 1022, assuming that the previously set next medical liquid infusion amount Vn is based on 100% efficacy, may anticipate that the efficacy will be reduced to the calculated changed efficacy value D, and calculate the compensatory infusion amount E. In addition, in operation 106a, the infusion amount determination unit 1022 may update the compensatory infusion amount E by using additional information received (in operation 106a) from the controller 20 or the integrated management server 30. Here, the additional information may be information about the medical liquid, such as a cumulative time during which the medical liquid has been stored in the storage unit, or information about a user, such as a site where the medical liquid infusion device is attached, a past absorption rate of the medical liquid for each site where the medical liquid infusion device was attached, a biometric value of the user, or emotional stress information about the user.

For example, the infusion amount determination unit 1022 may store a table table3 that includes, for each type of medical liquid, the degree to which efficacy is reduced according to a cumulative time during which the medical liquid has been stored in the storage unit 200. Each time a cumulative time during which insulin has been stored in the medical liquid storage unit 200 increases, the efficacy may decrease by 0.1%, and the compensatory infusion amount E may be updated by comparing the derived efficacy reduction value with the calculated next medical liquid infusion amount Vn. When the medical liquid has been stored in the storage unit for a long time, the compensatory infusion amount E may increase.

As another example, the infusion amount determination unit 1022 may store a table table4 including sites where the medical liquid infusion device 10 was attached, and past medical liquid absorption rates for the respective sites where the medical liquid infusion device 10 was attached. Past absorption rates of the medical liquid may be extracted for cases in which users attached the medical liquid infusion device 10 to their abdomen, thigh, or arm, and the compensatory infusion amount E may be updated by applying these absorption rates to the next medical liquid infusion amount Vn. For example, when a user attaches the medical liquid infusion device 10 to their abdomen, the medical liquid absorption rate may increase by 10% compared to when it is attached to their arm, and in this scenario, when the medical liquid infusion device 10 is attached to an arm, the compensatory infusion amount E may increase.

As yet another example, the medical liquid absorption rate may vary depending on information about the user's emotional stress. The infusion amount determination unit 1022 may store a table table5 in which users' degrees of emotional stress are matched with past medical liquid absorption rates. When the user experiences excessive emotional stress between the reference time and the next time, the compensatory infusion amount E may increase in response to the medical liquid absorption rate that is lowered due to the stress.

According to yet another embodiment of the disclosure, when the changed efficacy value D calculated in operation 105 is less than a threshold value L, the control module 1000 may alert the user with an alarm (operation 105a). Here, a situation where the changed efficacy value D is less than the threshold value L signifies that the cumulative efficacy reduction value has exceeded a certain level, that is, that the efficacy has decreased to such an extent that a desired effect cannot be obtained. In detail, when the changed efficacy value D calculated by the efficacy degradation rate calculation unit 1011 is less than the threshold value L, the integrated control unit 1012 may control the alarm unit 800 to provide, by using sound, light, vibration, or the like, an alarm to the user to whom the medical liquid infusion device 10 is attached (operation 105b). Here, the threshold value L may be a preset value, and for example, may be set such that when the changed efficacy value is less than 80%, 90%, or 95%, an alarm is provided. When the changed efficacy value D is less than the threshold value L, it may be a situation where the medical liquid has aged rapidly and replacement of the medical liquid is necessary. Thus, the control module 1000 notifies the user about a problem with the medical liquid through an alarm, to allow the user to respond quickly to the situation.

According to the above-described embodiment, it has been described that the control module 1000 changes the infusion amount of the medical liquid according to the changed efficacy value D calculated in operation 105, but the disclosure is not limited thereto. According to an optional embodiment of the disclosure, when the control module 1000 provides the user with the remaining amount of medical liquid stored in the storage unit, the control module 1000 may calculate the remaining amount of the medical liquid by considering the changed efficacy value D calculated in operation 105 and then provide the result value to the user. For example, when a first amount of medical liquid is actually stored in the storage unit but the changed efficacy value D is calculated to be lower than a complete efficacy of 1 due to exposure to high temperatures, the control module 1000 may provide the remaining amount of medical liquid stored in the storage unit to the user, as a second amount that is smaller than the first amount. The detailed calculation method has already been described by using Equation 3, and thus, redundant descriptions thereof will be omitted. Accordingly, the user may be provided with the remaining amount of medical liquid, considering whether the efficacy has decreased, and adjust the replacement time of the medical liquid infusion device, enabling a more accurate and stable medical liquid supply.

According to other embodiments of the disclosure, provided are a medical liquid infusion device and method that enable smart control, which considers not only infusing simply a preset amount of medical liquid but also an attachment state of the medical liquid infusion device and a biometric state of the user, by reflecting information about the medical liquid or information about the user in the next medical liquid infusion amount.

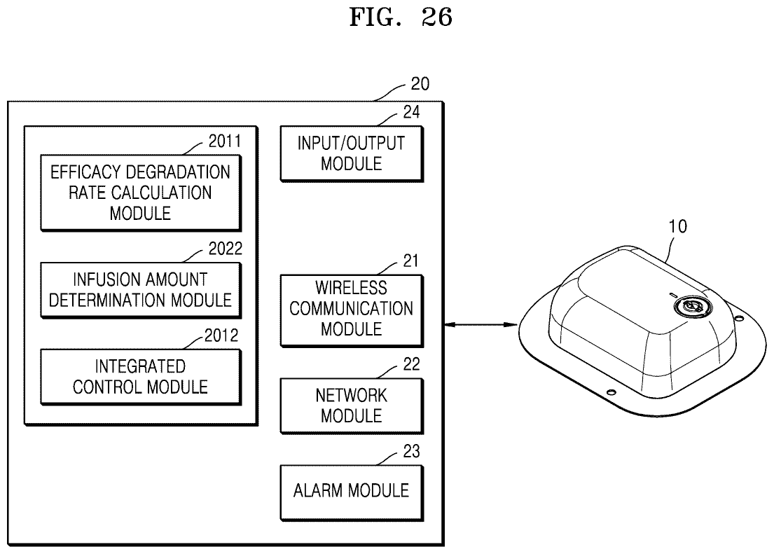

FIG. 26 is a block diagram illustrating a configuration of the controller 20 according to an embodiment of the disclosure.

The method of determining a medical liquid infusion amount according to an embodiment of the disclosure, as described above with reference to FIG. 19, may be performed by the medical liquid infusion device 10, but may also be performed by the controller 20 interworking with the medical liquid infusion device 10. The controller 20 may include a processing device such as a processor.

The controller 20 may include a wireless communication module 21, a network module 22, an input/output module 24, an alarm module 23, an efficacy degradation rate calculation module 2011, an infusion amount determination module 2022, and an integrated control module 2012. FIG. 12 illustrates only components associated with an embodiment of the disclosure, but it is apparent that the controller 20 may further include various components necessary for an operation of the controller 20, in addition to the illustrated components.

The wireless communication module 21 establishes a wireless communication connection for the controller 20 and the medical liquid infusion device 10 to transmit and receive signals, and the network module 22 establishes a network connection for the controller 20 and the integrated management server 30 to transmit and receive signals.

The input/output module 24 includes an input unit and an output unit, and may include an input unit for receiving information from the user via a keyboard, a keypad, a virtual keyboard, a touch display, buttons, a camera, or the like, and an output unit for outputting information to the user via a display, a speaker, light, a vibrator, or the like.

In response to a control signal, the alarm module 23 may output sound, light, or vibration, display a pop-up window on the input/output module, and when the changed efficacy value D calculated in operation 105 of FIG. 25 is less than the threshold value L, alert the user with an alarm.

The integrated control module 2012 controls each constituent component of the controller 20 based on various signals.

Similar to the efficacy degradation rate calculation unit 1011 of the medical liquid infusion device illustrated in FIG. 18, the efficacy degradation rate calculation module 2011 receives, from a medical liquid infusion device, a signal indicating a medical liquid temperature measured at every unit time interval t during an interval from a reference time ts to a next time tn, calculates a cumulative efficacy reduction value for the temperature that changes for an elapsed time r between the reference time ts and the next time tn, and then derives the changed efficacy value D that indicates the degree to which the efficacy has decreased, by comparing the calculated cumulative efficacy reduction value with a reference efficacy value I. (operations 102 to 105 of FIG. 19)

The infusion amount determination module 2022 calculates a compensatory infusion amount E based on the derived changed efficacy value D and the previously set next medical liquid infusion amount Vn, and derives a corrected medical liquid infusion amount V′n in which this compensatory infusion amount E is reflected. (operations 106 to 107 of FIG. 19) According to another embodiment, the infusion amount determination module 2022 may update the compensatory infusion amount E by using, as factors, additional information in addition to the next medical liquid infusion amount Vn and the changed efficacy value D, and derive a corrected medical liquid infusion amount V′n in which the updated compensatory infusion amount E is reflected. (operations 106a to 107 of FIG. 25)

Although not illustrated, according to another embodiment of the disclosure, the method of determining a medical liquid infusion amount according to an embodiment of the disclosure, as described above with reference to FIG. 19, may be performed by the medical liquid infusion device 10 or the controller 20, but may also be performed by the integrated management server 30. In this case, the integrated management server 30 may include constituent components necessary for determining a medical liquid infusion amount, and may receive, from the medical liquid infusion device 10 or the controller 20, data necessary for deriving a corrected medical liquid infusion amount.

The disclosure discloses various embodiments of a method of controlling a medical liquid infusion device. Hereinafter, descriptions will be provided with reference to FIGS. 27 to 33.

FIG. 27 is a block diagram illustrating a schematic configuration of the controller 20 according to an embodiment of the disclosure.

The controller 20 may include a first wireless communication module 21, a second wireless communication module 22, a network module 23, an input/output module 24, a signal type determination module 25, and an encryption module 26. FIG. 27 illustrates only components associated with an embodiment of the disclosure, but the controller 20 may further include various components necessary for an operation of the controller 20, in addition to the illustrated components. In addition, for convenience of description, the illustrated constituent components are distinguished and illustrated according to their roles or operations, and one constituent component may be combined with another constituent component into a single constituent component, or one constituent component may be separated into several distinct constituent components.

The first wireless communication module 21 establishes a connection for first wireless communication enabling the controller 20 and the medical liquid infusion device 10 to transmit and receive a first signal. In response to a new device registration request from a user desiring to register a new medical liquid infusion device 10, the first wireless communication module 21 performs scanning for nearby medical liquid infusion devices 10, transmits a connection request signal to the medical liquid infusion device 10, and completes the first wireless communication connection by transmitting and receiving connection parameters when the wireless connection starts. The first wireless communication module 21 is able to establish a connection when the medical liquid infusion device 10 is within a first distance (e.g., within about 1 m) from the controller 20, and the connection between the two connected devices is maintained until it is released. For example, the first wireless communication module may be a Bluetooth or Bluetooth Low Energy module.

The second wireless communication module 22 establishes a connection for second wireless communication enabling the controller 20 and the medical liquid infusion device 10 to transmit and receive a second signal. In response to the controller 20 approaching the medical liquid infusion device 10 within a second distance, the second wireless communication module 22 performs a connection between the two devices. Here, the second distance (e.g., about several tens of centimeters) is shorter than the first distance. For example, the second wireless communication module may be an NFC module capable of NFC reading and writing.

According to an embodiment of the disclosure, first signals transmitted by the first wireless communication module 21 are different from second signals transmitted by the second wireless communication module 22. According to an embodiment, the second signals transmitted by the second wireless communication module 22 include infusion signals. On the contrary, the first signals transmitted by the first wireless communication module 21 include control signals and data, other than infusion signals. According to another embodiment, the second signals transmitted by the second wireless communication module 22 include only specific infusion signals. On the contrary, the first signals transmitted by the first wireless communication module 21 include other infusion signals than the above-described specific infusion signals, all general control signals, and data.

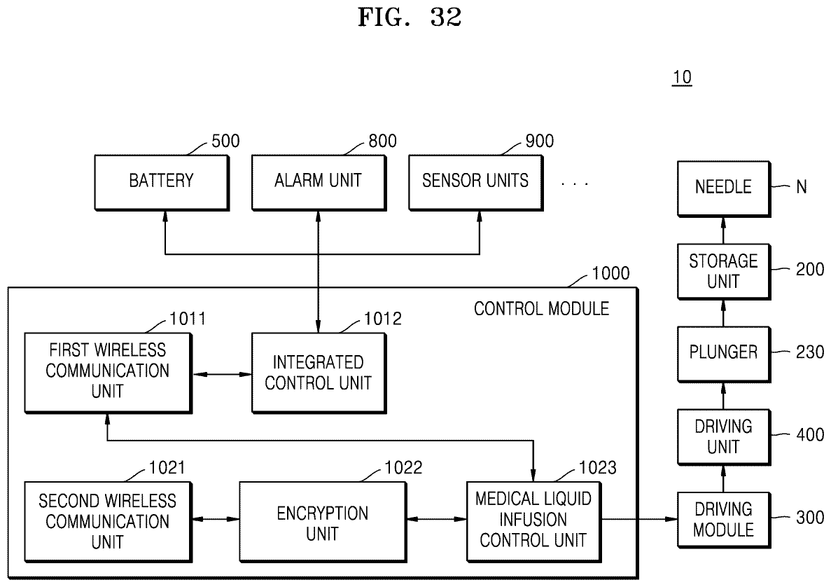

In detail, an infusion signal according to an embodiment is a command for controlling the driving module 300 (see FIG. 32) in the medical liquid infusion device 10 to infuse a medical liquid stored in the storage unit 200 (see FIG. 32), into a subject through the needle N (see FIG. 32). In addition, an infusion signal refers to any signal that varies an infusion amount, such as signals for starting, changing, canceling, or stopping the infusion of a medical liquid. Infusion signals may include an infusion signal by bolus injection, an infusion signal according to a basic infusion program, a remote infusion signal from the integrated management server 30, and the like. In addition, control signals may include, but are not limited to, commands for controlling the medical liquid infusion device 10, such as a signal for controlling the alarm unit 800 (see FIG. 32) of the medical liquid infusion device 10, a signal for controlling the sensor units 900 (see FIG. 32), or a signal for controlling a battery 500 (see FIG. 32). In addition, herein, data may be device data from the medical liquid infusion device 10, such as a medical liquid infusion history, an amount of medical liquid stored in the storage unit 200 (see FIG. 32), a remaining battery capacity, whether an injection port is occluded, or whether there is a device abnormality, or may be biometric values of a user measured by the medical liquid infusion device 10, such as a blood glucose level, a blood pressure, or a heart rate.

In detail, in another embodiment, a specific infusion signal transmitted by the second wireless communication module 22 refers to an infusion signal of a first type. First, an infusion signal according to an embodiment is a command for controlling the driving module 300 (see FIG. 32) in the medical liquid infusion device 10 to infuse a medical liquid stored in the storage unit 200 (see FIG. 32), into a subject through the needle N (see FIG. 32). Here, according to an embodiment, an infusion signal of the first type may be an infusion signal by bolus injection, and according to another embodiment, it may be a remote infusion signal that is input to the controller 20 through the integrated management server 30.

On the contrary, a specific signal transmitted by the first wireless communication module 21 refers to infusion signals other than infusion signals of the first type, all control signals, and data. Here, the infusion signals other than infusion signals of the first type may include infusion signals of a second type. Here, infusion signals of the second type may be infusion signals according to a basic infusion program. Other general control signals may include all control signals necessary for controlling the medical liquid infusion device 10, excluding those for medical liquid infusion. For example, the other general control signals may include, but are not limited to, a signal for controlling the alarm unit 800 (see FIG. 32) of the medical liquid infusion device 10, a signal for controlling the sensor units 900 (see FIG. 32), or a signal for controlling the battery 500 (see FIG. 32). In addition, herein, data may be device data from the medical liquid infusion device 10, such as a medical liquid infusion history, an amount of medical liquid stored in the storage unit 200 (see FIG. 32), a remaining battery capacity, whether an injection port is occluded, or whether there is a device abnormality, or may be biometric values of a user measured by the medical liquid infusion device 10, such as a blood glucose level, a blood pressure, or a heart rate.

Hereinafter, infusion signals of the first type and infusion signals the second type will be described in more detail.

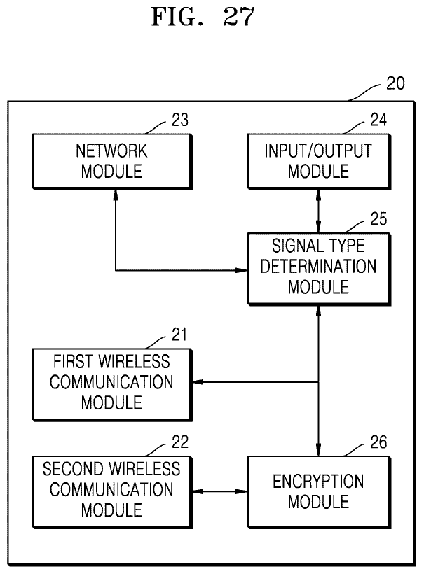

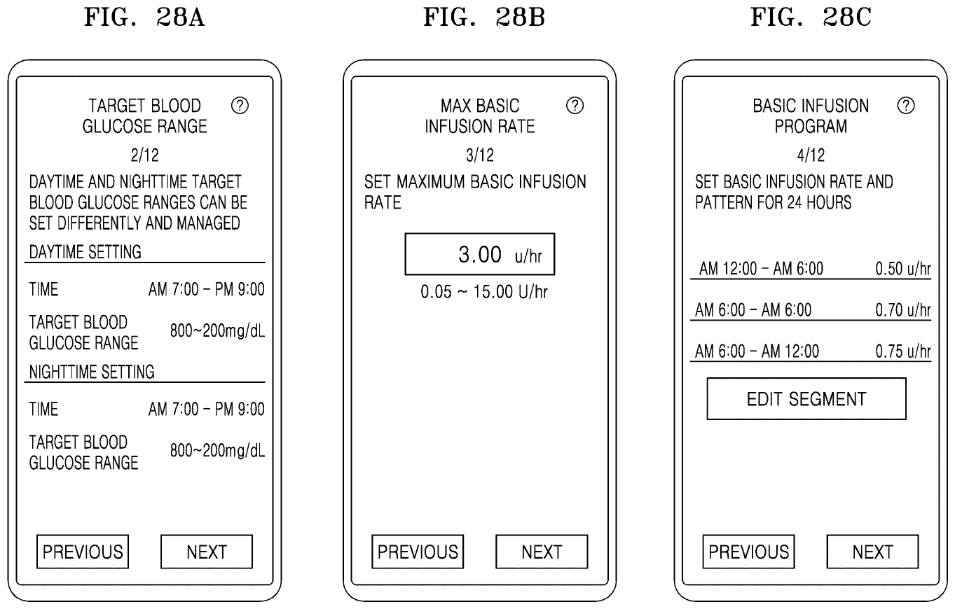

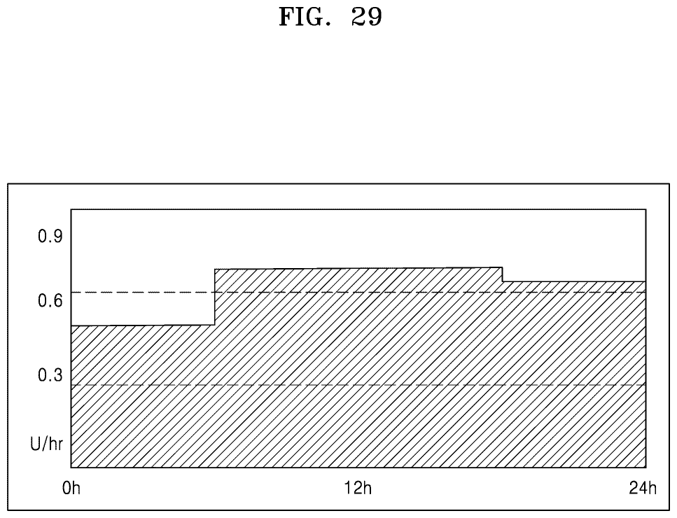

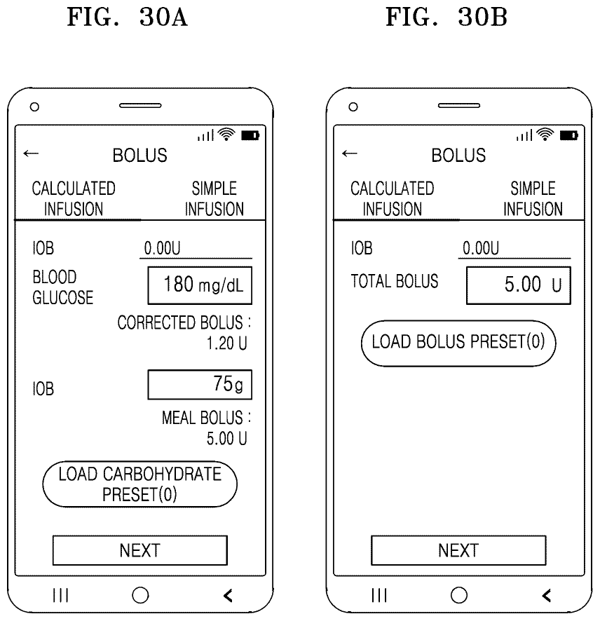

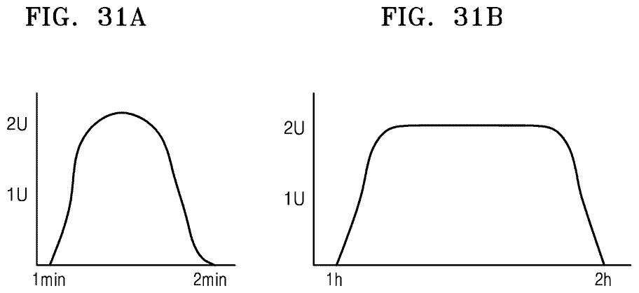

FIGS. 28A to 28C illustrate input/output screens of the controller 20 for explaining a medical liquid infusion method according to a basic infusion program of the controller 20. FIGS. 29A and 29B are graphs illustrating an amount of medical liquid infused by time according to the basic infusion program of FIGS. 28A to 28C. FIGS. 30A and 30B illustrate input/output module screens of the controller 20 for explaining a medical liquid infusion method according to bolus injection of the controller 20. FIGS. 31A and 31B are graphs illustrating an amount of medical liquid infused over time by the bolus injection of FIGS. 30A and 30B.

The controller 20 may implement a medical liquid infusion method according to a basic infusion program or bolus injection.

Referring to FIGS. 28A to 28C and FIG. 29, the basic infusion program is an infusion method that schedules in advance for a user-set amount of medical liquid to be infused at a user-set rate at a user-specified time during a preset period (e.g., for 24 hours). Referring to FIG. 28A, the user may set a target blood glucose range through the input/output module 24 of the controller 20. That is, daytime and nighttime periods may be defined, and then a target blood glucose range to be achieved in each time period may be set. Because blood glucose levels are likely to remain high during the day due to food consumption, a target blood glucose range different from that for the nighttime may be set to lower blood glucose accordingly and maintain it within a certain range. Referring to FIG. 28B, the user may set a maximum basic infusion rate of a medical liquid, and referring to FIG. 28C, the user may set different basic infusion rates for different time periods. Because blood glucose may rise rapidly after food consumption, setting different basic infusion rates for different time periods in conjunction with meal or snack times helps to keep the blood glucose level within a normal blood glucose range.

Referring to FIGS. 29A and 29B, according to the basic infusion program that is set in FIGS. 28A to 28C, the medical liquid is infused in amounts and at rates (U/hr) that are set for the respective time periods. For example, it may be confirmed that a relatively small amount of medical liquid is set to be infused during the night or before morning, and that a relatively large amount of medical liquid is set to be infused from after morning until evening. Here, U on the vertical axis of FIGS. 29A and 29B denotes a unit, where 1 unit is 1/24 mg of purified insulin, and 1 mg of insulin signifies 24 units. The horizontal axis represents time (hour).

Infusion signals based on the target blood glucose range for each time period, the maximum basic infusion rate, and the basic infusion rate for each time period, which are set according to the basic infusion program, are defined as infusion signals of the second type. For infusion signals of the second type, the user may directly set the basic infusion program through the controller 20, as illustrated in FIG. 3. Infusion signals of the second type that are set in this manner may be transmitted from the controller 20 to the medical liquid infusion device 10 through the first wireless communication.

Next, referring to FIGS. 30A, 30B, 31A, and 31B, bolus injection is an infusion method, which is different from basic infusion, in which a medical liquid is infused immediately whenever necessary, either to lower blood glucose that rises postprandially due to food intake or to correct high blood glucose to a target blood glucose level. As illustrated in FIG. 30A, the user may set an infusion amount of medical liquid according to a carbohydrate intake amount through the input/output module of the controller 20, and, as illustrated in FIG. 30B, the user may also directly set an infusion amount of medical liquid to be infused. Because blood glucose may rise rapidly, especially after consuming carbohydrates, bolus injection is performed after consuming food high in carbohydrates to enable a rapid return to within the normal blood glucose range.

Referring to FIGS. 31A and 31B, unlike FIGS. 29A and 29B described above, according to the bolus injection set in FIGS. 30A and 30B, the medical liquid is infused in amounts and at rates that are set for a short period (e.g., several minutes (min) as illustrated in FIG. 31A, or several hours as illustrated in FIG. 31B).

An infusion signal that commands the immediate infusion of a medical liquid, based on the medical liquid infusion amount set according to the bolus injection in this manner, is defined as an infusion signal of the first type. For an infusion signal of the first type, the user may also directly set bolus injection through the controller 20, as illustrated in FIGS. 30A and 30B. Infusion signals of the first type that are set in this manner may be transmitted from the controller 20 to the medical liquid infusion device 10 through the second wireless communication.

According to an optional embodiment, a user, medical staff, or a guardian may set the basic infusion program illustrated in FIGS. 28A to 28C or the bolus injection illustrated in FIGS. 30A and 30B by using a web or an application provided by the integrated management server 30. Even in this case, it is a matter of course that an infusion signal based on the basic infusion program set on the integrated management server 30 is transmitted to the medical liquid infusion device 10 via the controller 20. An infusion signal set in this manner by using the integrated management server 30 is defined as an infusion signal of the first type, and this infusion signal of the first type may be transmitted from the controller 20 to the medical liquid infusion device 10 through the second wireless communication.

Infusion signals that are directly related to the user's life and health must be subjected to thorough security management. When infusion signals are transmitted exclusively by the first wireless communication, the user is not free from the threat of malicious hacking. This is because the first wireless communication enables communication between devices within a radius of several tens of meters, rendering it vulnerable to hacking. Therefore, there is a concern that the user may not receive a medical liquid infusion when necessary or, conversely, may receive an excessive medical liquid infusion when it is not necessary.

According to an embodiment of the disclosure, by enabling infusion signals for which security is important to be transmitted to the medical liquid infusion device 10 via the second wireless communication rather than the first wireless communication, an effect of enhancing security and promoting user safety is achieved.

Furthermore, according to another embodiment of the disclosure, by enabling infusion signals of a specific type, for which the importance of security is particularly emphasized, to be transmitted to the medical liquid infusion device 10 via the second wireless communication rather than the first wireless communication, an effect of enhancing user safety is achieved.

In detail, an infusion signal of the first type transmitted via the second wireless communication may be an infusion signal that commands the immediate infusion of a medical liquid based on a medical liquid infusion amount set according to bolus injection, or may be a remote infusion signal set on the integrated management server 30 and then input to the controller 20. This is because, firstly, in the case of an infusion signal set according to bolus injection, the medical liquid may be immediately infused into a subject, and thus, there is a concern that the subject's health may be threatened by malicious attacks.

Furthermore, in the case of a remote infusion signal set by the integrated management server 30, the remote infusion signal originates from a source physically distant from the controller 20 and the medical liquid infusion device 10, and there is a concern of hacking by a third party, and thus, the remote infusion signal may be considered more vulnerable in terms of security compared to an infusion signal directly input by the user to the controller 20.

A connection for the second wireless communication is established only when the two devices come into significantly close proximity. In other words, only when the controller 20 is tapped against the medical liquid infusion device 10 attached to the user, an infusion signal of the first type may be transmitted to the medical liquid infusion device 10. Therefore, this has the effect of resolving the security problem that is a concern with the first wireless communication.

In addition, an infusion signal of the second type, for example, an infusion signal directly set by the user on the controller 20 and based on a target blood glucose range for each time period, a maximum basic infusion rate, and a basic infusion rate for each time period that are set according to the basic infusion program, is transmitted from the controller 20 to the medical liquid infusion device 10 through the first wireless communication, because the infusion signal has been already scheduled, and thus, access control of the infusion signal is more difficult than that for infusion signals of the first type.

In addition, general control signals other than infusion signals, for example, a signal for controlling the alarm unit of the medical liquid infusion device 10, a signal for controlling the sensor units, or a signal for controlling the battery, are transmitted from the controller 20 to the medical liquid infusion device 10 through the first wireless communication, similarly to infusion signals of the second type, because even when they become targets of malicious attacks or hacking, the direct risk to the subject to whom the medical liquid infusion device 10 is attached is less compared to that from compromised infusion signals.

Furthermore, data, for example, device data from the medical liquid infusion device 10, such as a medical liquid infusion history, an amount of medical liquid stored in the storage unit, a remaining battery capacity, whether an injection port is occluded, or whether there is a device abnormality, or biometric values of the user measured by the medical liquid infusion device 10, such as a blood glucose level, a blood pressure, or a heart rate, has a relatively lower security level compared to infusion signals and is thus transmitted from the controller 20 to the medical liquid infusion device 10 through the first wireless communication, similarly to infusion signals of the second type.

Referring back to FIG. 27, the network module 23 establishes a network connection enabling the controller 20 and the integrated management server 30 to transmit and receive signals. The network module 23 may receive signals from the integrated management server 30. Here, the signals may include infusion signals, which may be generated by medical staff or a guardian, in addition to the user, by using a web or an application provided by the integrated management server 30.

The input/output module 24 includes an input unit and an output unit, and may include an input unit for receiving information from the user via a keyboard, a keypad, a virtual keyboard, a touch display, buttons, a camera, or the like, and an output unit for outputting information to the user via a display, a speaker, light, a vibrator, or the like.

The signal type determination module 25 determines whether to use the first wireless communication module or the second wireless communication module when a signal received from the network module 23, or a signal input through the input/output module 24, is transmitted to the medical liquid infusion device 10. In detail, according to an embodiment, the signal type determination module 25 receives a signal and first determines whether the input signal is an infusion signal. In addition, according to another embodiment, the signal type determination module 25 receives a signal, first determines whether the input signal is an infusion signal, and when the signal is determined as an infusion signal, determines what type of infusion signal it is.

First, the signal type determination module 25 determines whether a signal received from the network module 23 and a signal input through the input/output module 24 are infusion signals. When the signal is not an infusion signal but a general control signal, the signal type determination module 25 transmits the corresponding control signal to the first wireless communication module 21. When the signal is not an infusion signal but is data, the signal type determination module 25 stores the data in a storage unit (not shown) or inputs the data to a processor (not shown) for use in controlling the controller 20.

When the determination result indicates that the signal is an infusion signal, the signal type determination module 25 determines whether the type of the infusion signal is the first type or the second type. When the infusion signal is of the first type, the signal type determination module 25 transmits the infusion signal of the first type to the second wireless communication module 22. When the infusion signal is of the second type, the signal type determination module 25 transmits the infusion signal of the second type to the first wireless communication module 21.

The signal type determination module 25 may store a table in which types of signals are indicated, and may determine the type of an input signal by comparing the input signal with the table, and this configuration may be stored as a plurality of algorithms.

The encryption module 26 performs encryption for additional security of signals transmitted by using the second wireless communication. Here, the signal may be an infusion signal according to an embodiment, or may be an infusion signal of the first type according to another embodiment. In detail, the encryption module may include an encryption/decryption unit, a storage unit, a key management unit, and the like, which are not illustrated. The key management unit generates or extracts an encryption key to be exchanged with the medical liquid infusion device 10 through the second wireless communication. The encryption/decryption unit encrypts, by using the extracted encryption key, an infusion signal or an infusion signal of the first type, which is to be transmitted through the second wireless communication. The storage unit stores the encrypted infusion signal or the encrypted infusion signal of the first type. The stored infusion signal or the stored infusion signal of the first type is transmitted to the medical liquid infusion device 10 through the second wireless communication.

Each time a new medical liquid infusion device 10 is registered, the key management unit may randomly generate or extract a new encryption key in accordance with the registration. However, the disclosure is not limited thereto, the user may arbitrarily set an encryption key in the form of a private key.

The encryption/decryption unit encrypts an infusion signal or an infusion signal of the first type by using an extracted encryption key and stores the result in the storage unit, and this encryption may be performed by using an encryption algorithm that is any one of various encryption methods such as a symmetric encryption method or an asymmetric encryption method.

According to an embodiment of the disclosure, user safety may be enhanced by enabling an infusion signal or an infusion signal of the first type, for which the importance of security is emphasized, to be transmitted to the medical liquid infusion device 10 via the second wireless communication rather than the first wireless communication. Furthermore, an encryption process is additionally performed on the infusion signal or the infusion signal of the first type, enabling further enhanced security. For example, when an infusion signal or an infusion signal of the first type does not undergo an encryption process, there is a concern that a third party may intentionally or unintentionally tap the controller 20 against the medical liquid infusion device 10 to infuse a drug into the user. However, according to an embodiment of the disclosure, by encrypting an infusion signal or an infusion signal of the first type by using an encryption key, the infusion signal or the infusion signal of the first type may actually operate only for the controller 20 and the medical liquid infusion device 10 between which the encryption key has already been exchanged via a key exchange method, and infusion of a medical liquid by an unauthorized third party may be prevented.

FIG. 32 is a block diagram schematically illustrating a control module of the medical liquid infusion device 10, and constituent components associated with the control module.

The medical liquid infusion device 10 may include the needle N, the storage unit 200, the plunger 230, the driving module 300, the driving unit 400, the alarm unit 800, the plurality of sensor units 900 including a temperature sensor, and the control module 1000. For the needle N, the storage unit 200 (201 of FIG. 4), the plunger 230, the driving module 300, the driving unit 400, the alarm unit 800, and the sensor units 900, reference is made to the descriptions provided above with reference to FIGS. 4 and 18.

Referring to FIG. 32, the control module 1000 may include a first wireless communication unit 1011, a second wireless communication unit 1021, an encryption unit 1022, the medical liquid infusion control unit 1023, and the integrated control unit 1012. FIG. 32 illustrates only constituent components associated with an embodiment of the disclosure, but the controller 20 may further include various constituent components necessary for an operation of the controller 20, in addition to the illustrated constituent components.

The first wireless communication unit 1011 establishes a connection for first wireless communication enabling the controller 20 and the medical liquid infusion device 10 to transmit and receive infusion signals, other than infusion signals according to an embodiment or infusion signals of the first type according to another embodiment. In addition, the first wireless communication unit 1011 establishes a connection for first wireless communication for transmitting and receiving general control signals, data, and the like. The first wireless communication unit 1011, upon a medical liquid being injected into the storage unit 200, i.e., triggered by the medical liquid injection, performs advertising to transmit an advertising message corresponding to a pairing request signal, receives a connection request signal from the controller 20, and completes the first wireless communication connection by transmitting and receiving connection parameters once the wireless connection starts. For example, the first wireless communication unit 1011 may be a Bluetooth or Bluetooth Low Energy module.

The second wireless communication unit 1021 establishes a connection for second wireless communication enabling the controller 20 and the medical liquid infusion device 10 to transmit and receive infusion signals according to an embodiment or infusion signals of the first type according to another embodiment. In response to the controller 20 approaching the medical liquid infusion device 10 within a second distance, the second wireless communication unit 1021 performs a connection between the two devices. Here, the second distance (e.g., about several tens of centimeters) is shorter than the first distance. For example, the second wireless communication unit 1021 may be an NFC module.

The integrated control unit 1012 controls each constituent component of the medical liquid infusion device 10 based on various signals. For example, the integrated control unit 1012 may control, by using a signal that controls the alarm unit 800, the on/off state and the like of the alarm unit of the medical liquid infusion device 10, and by using a signal that controls the sensor units 900, may detect whether there is a device abnormality, and measure the user's blood glucose level, blood pressure, heart rate, and the like, and by using a signal that controls the battery 700 (see FIG. 4), may measure the remaining capacity of the battery. Based on these processes, the integrated control unit 1012 may generate device data and biometric values.

According to an embodiment, the medical liquid infusion control unit 1023, based on an infusion signal received through the second wireless communication unit 1021, controls the driving module 300 such that the medical liquid stored in the storage unit 200 is discharged through the needle N, as described above with reference to FIG. 7. In addition, according to another embodiment, based on an infusion signal of the first type received through the second wireless communication unit 1021 and an infusion signal of the second type received through the first wireless communication unit 1011, the medical liquid infusion control unit 1023, controls the driving module 300 such that the medical liquid stored in the storage unit 200 is discharged through the needle N.

The encryption unit 1022 performs decryption of an encrypted infusion signal or an encrypted infusion signal of the first type. In detail, similar to the encryption module of the controller 20, the encryption unit 1022 may include an encryption/decryption unit, a storage unit, a key management unit, and the like, which are not illustrated. The key management unit extracts or generates an encryption key to be exchanged with the controller 20 through the second wireless communication. The encryption/decryption unit decrypts, by using the exchanged encryption key, an infusion signal or an infusion signal of the first type received through the second wireless communication. The storage unit stores the decrypted infusion signal or the decrypted infusion signal of the first type. The stored infusion signal or the stored infusion signal of the first type is transmitted to the medical liquid infusion control unit 1023 to control a medical liquid infusion.

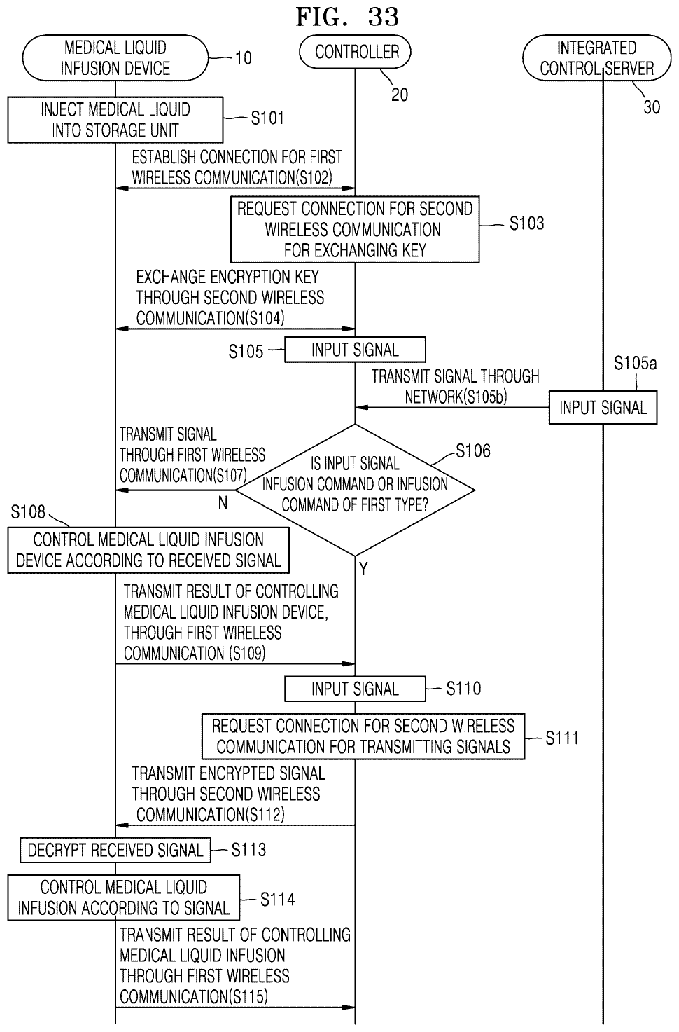

FIG. 33 is a flowchart illustrating a method of controlling the medical liquid infusion device 10, according to an embodiment of the disclosure.

Referring to FIG. 33, in operation 101, a medical liquid is injected into the storage unit 200 (see FIG. 32) of the medical liquid infusion device 10. The user may insert a syringe into a medical liquid injection port of an attachment portion of the medical liquid infusion device 10 to fill the storage unit 200 (see FIG. 32) of the medical liquid infusion device 10 with a medical liquid.

In operation 102, a connection for first wireless communication is established between the medical liquid infusion device 10 and the controller 20. In detail, the medical liquid infusion device 10, upon a medical liquid being injected, i.e., triggered by the medical liquid injection, performs advertising to transmit an advertising message corresponding to a pairing request signal. In response to a new device registration request from a user desiring to register a new medical liquid infusion device 10, the controller 20 performs scanning for nearby medical liquid infusion devices 10, and transmits a connection request signal to the medical liquid infusion device 10. When the medical liquid infusion device 10 receives the connection request signal from the controller 20, a wireless connection starts, and the two devices complete the first wireless communication connection by transmitting and receiving connection parameters. As described above, the first wireless communication connection may be a Bluetooth or Bluetooth Low Energy connection.

In operation 103, the controller 20 requests a connection for second wireless communication for exchanging an encryption key. When the first wireless communication connection is established between the two devices, the controller 20 extracts or generates an encryption key. When the encryption key is extracted or generated, the controller 20 generates a sound, a light, a guidance pop-up, or the like through the input/output module 24, and requests the user to establish a connection for second wireless communication with the medical liquid infusion device 10 for exchanging the encryption key. In addition, the medical liquid infusion device 10 also extracts or generates an encryption key when the first wireless communication connection with the controller 20 is established.

In operation 104, the controller 20 exchanges the encryption key with the medical liquid infusion device 10 through the second wireless communication. Here, the second wireless communication connection is performed in response to the controller 20 approaching within a second distance of, or tapping against, the medical liquid infusion device 10. Here, the second wireless communication may be performed based on an NFC method. The encryption key exchanged between the two devices is stored in both devices and is used for transmitting encrypted signals and for their decryption, thereby enabling a high level of security to be maintained concerning signal transmission.

In operation 105, the controller 20 receives a signal from the user. The controller 20 may directly receive a signal from the user through the input/output module 24, however, according to an optional embodiment, as in operation 105a, a signal may be received from the user or a third party such as medical staff or a guardian, via a web or an application provided by the integrated management server 30, and then transmitted to the controller 20 through a network. Here, the signal may include an infusion signal and a general control signal other than infusion signals. The infusion signal may include an infusion signal of the first type and an infusion signal of the second type. Infusion signals of the first type may include infusion signals according to bolus injection, and according to an optional embodiment, may include remote infusion signals input to the controller 20 through the integrated management server 30. Infusion signals of the second type may refer to all infusion signals other than infusion signals of the first type. General control signals may include signals for controlling the medical liquid infusion device 10, other than infusion signals.

In operation 106, the controller 20 determines whether the received signal is an infusion command (according to an embodiment) or an infusion command of the first type (according to another embodiment). In detail, the controller 20 needs to determine whether to use the first wireless communication, or to use the second wireless communication after encryption, when the signal is transmitted to the medical liquid infusion device 10. According to an embodiment, the controller 20 determines whether the input signal is an infusion signal. According to another embodiment, the controller 20 first determines whether the input signal is an infusion signal, and when the input signal is determined as an infusion signal, determines what type of infusion signal it is.

When the signal is not an infusion signal but is a general control signal, the controller 20 transmits the control signal to the medical liquid infusion device 10 through the first wireless communication. (S107) When the signal is neither an infusion signal nor a control signal but is data, the controller 20 may store the data in a storage unit (not shown) or input the data to a processor (not shown) for use in controlling the controller 20.

When the signal is an infusion signal, then according to an embodiment, the controller 20 transmits the infusion signal to the medical liquid infusion device 10 through the second wireless communication (S107). In another embodiment, when the signal is an infusion signal, the controller 20 determines whether the type of the infusion signal is the first type or the second type. When the infusion signal is of the second type, the controller 20 transmits the infusion signal of the second type to the medical liquid infusion device 10 through the first wireless communication. (S107)

The medical liquid infusion device 10 may control the medical liquid infusion device 10 according to the signal transmitted in operation 107 (S108), and transmit a result of controlling the medical liquid infusion device 10, as data to the controller 20 through the first wireless communication. (Operation S109) For example, in another embodiment, when an infusion signal of the second type is transmitted through the first wireless communication, and the signal transmitted in operation 107 is of the second type, the medical liquid infusion device 10 may perform medical liquid infusion based on a target blood glucose range for each time period, a maximum basic infusion rate, and a basic infusion rate for each time period, which are preset by the basic infusion program, according to the infusion signal of the second type, and transmit a result of the infusion to the controller 20. As another example, when the signal transmitted in operation 107 is a general control signal, the medical liquid infusion device 10 may transmit, to the controller 20, device data such as a medical liquid infusion history, an amount of medical liquid stored in the storage unit, a remaining battery capacity, whether an injection port is occluded, or whether there is a device abnormality, which is a result of monitoring the storage unit, the alarm unit, the sensor units, or the battery, or biometric values of the user, such as a measured blood glucose level, blood pressure, or heart rate.

Returning to operation 106, in an embodiment, when the signal is an infusion signal, the controller 20 encrypts the infusion signal of the first type by using the encryption key exchanged in operation 104. Because infusion signals require enhanced security compared to other control signals or data, user safety may be promoted through additional security procedures. In another embodiment, when the signal is an infusion signal and the infusion signal is of the first type, the controller 20 encrypts the infusion signal of the first type by using the encryption key exchanged in operation 104. (Operation S110) Here, because the infusion signal of the first type is a signal that may have an immediate effect on the user's body or may be input by a third party other than the user, user safety may be protected by introducing additional security procedures.

In operation 111, the controller 20 requests a connection for second wireless communication for transmitting signals. In detail, when the encryption is completed, the controller 20, by using the input/output module to generate a sound, a light, a guidance pop-up, or the like, requests the user to establish a connection for second wireless communication with the medical liquid infusion device 10 for transmitting the encrypted signal.

In operation 112, when the user brings the controller 20 to approach within a second distance from the medical liquid infusion device 10, or taps the controller 20 against the medical liquid infusion device 10, the controller 20 transmits the encrypted signal to the medical liquid infusion device 10 through the second wireless communication.

In operation 113, the medical liquid infusion device 10 decrypts the received signal. The medical liquid infusion device 10 decrypts the signal by using the encryption key exchanged in operation 104.

In operation 114, the medical liquid infusion device may control medical liquid infusion according to the decrypted signal (S114), and transmit a result of controlling the medical liquid infusion as data to the controller 20 through the first wireless communication. (Operation S115) For example, the medical liquid infusion device 10 may perform, according to the decrypted infusion signal of the first type, immediate medical liquid infusion based on a medical liquid infusion amount set according to bolus injection, and transmit a result of the infusion to the controller 20. In addition, the medical liquid infusion device 10, according to the decrypted infusion signal of the first type, may perform medical liquid infusion in a method that is input through the integrated management server 30, and transmit a result of the infusion to the controller 20.

According to an embodiment of the disclosure, all infusion signals, such as infusion signals of the first type, infusion signals of the second type, and remote infusion signals, may be transmitted to the medical liquid infusion device through the second wireless communication, while control signals and data, other than infusion signals, may be transmitted to the medical liquid infusion device 10 through the first wireless communication. Accordingly, transmission of all infusion signals may be performed by using a security-enhanced method.

In addition, according to another embodiment of the disclosure, by enabling infusion signals of a specific type, for which the importance of security is particularly emphasized, to be transmitted to the medical liquid infusion device 10 via the second wireless communication rather than the first wireless communication, an effect of enhancing user safety is achieved. That is, a connection for the second wireless communication is established only when the two devices come into significantly close proximity. In other words, only when the controller 20 is tapped against the medical liquid infusion device 10 attached to the user, an infusion signal of a specific type may be transmitted to the medical liquid infusion device 10. Therefore, this has the effect of resolving the security vulnerability from short-range hacking that may occur when the first wireless communication is used.

According to an embodiment of the disclosure, by adding an operation of encrypting an infusion signal of a specific type through an encryption key exchange via the second wireless communication, the effect of further enhancing security is achieved. That is, to protect the user from a third party maliciously and forcibly bringing the two devices into close proximity to transmit an infusion signal of a specific type to the medical liquid infusion device 10, a feature of the disclosure is that, by enabling only two devices that have exchanged an encryption key to use a specific infusion signal, a security vulnerability, which arises from malicious and forcible acts that may occur when the second wireless communication is available, is resolved.

Meanwhile, according to an optional embodiment of the disclosure, operations 103, 104, 110, and 113 may be omitted. In this case, as additional security procedures are omitted, although the security level for infusion signals of the second type may be lowered, there is the effect of increased user convenience because the user does not need to perform tapping of the two devices for encryption key exchange.

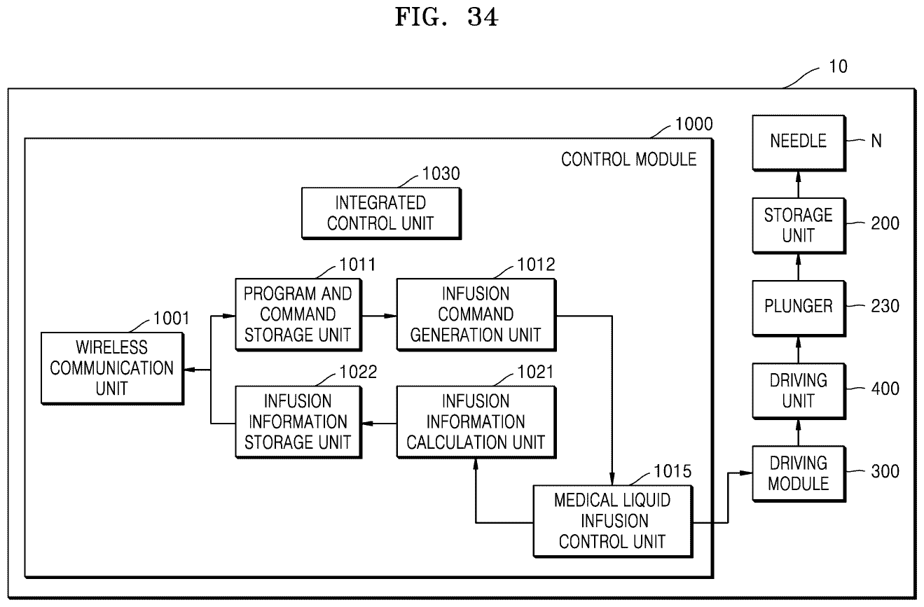

The disclosure discloses various embodiments of a method, apparatus, and a computer program product for displaying a medical liquid infusion result of a medical liquid infusion device when a wireless communication-related event occurs. Hereinafter, descriptions will be provided with reference to FIGS. 34 to 57.

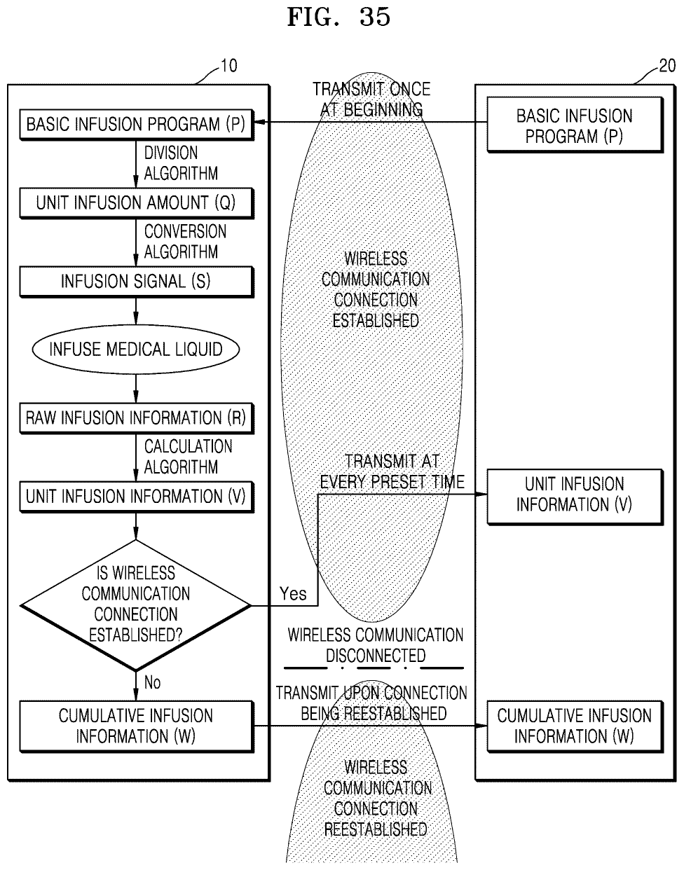

FIG. 34 is a block diagram schematically illustrating a control module of the medical liquid infusion device 10, and constituent components associated with the control module. FIG. 35 is a conceptual diagram schematically illustrating a flow of signals, data, and information that are received, transmitted, and generated by constituent components of the medical liquid infusion device 10 of FIG. 34.

Referring to FIGS. 34 and 35, the control module 1000 may include a wireless communication unit 1001, a program and command storage unit 1011, an infusion command generation unit 1012, a medical liquid infusion control unit 1015, an infusion information calculation unit 1021, an infusion information storage unit 1022, and an integrated control unit 1030. FIG. 34 illustrates only constituent components associated with an embodiment of the disclosure, but the control module 1000 may further include various constituent components necessary for an operation of the medical liquid infusion device 10, in addition to the illustrated constituent components. Each constituent component included in the control module 1000 may be operated by a processing device designated as the integrated control unit 1030, such as a processor.

The wireless communication unit 1001 establishes a wireless communication connection enabling the controller 20 and the medical liquid infusion device 10 to transmit and receive various signals and data. The wireless communication unit 1001, upon a medical liquid being injected into the storage unit 200, i.e., triggered by the medical liquid injection, performs advertising to transmit an advertising message, receives a connection request signal from the controller 20, and completes the wireless communication connection by transmitting and receiving connection parameters once the wireless connection starts. For example, the wireless communication unit 1001 may be a Bluetooth or Bluetooth Low Energy module.

The wireless communication may maintain its connection when the medical liquid infusion device 10 and the controller 20 are present within a certain distance of each other. Here, the certain distance may be from several to several tens of meters, and may preferably be about 10 m or less. When the distance between the medical liquid infusion device 10 and the controller 20 is greater than the certain distance, the wireless communication connection is automatically released. When the wireless communication connection is released, the medical liquid infusion device 10 and the controller 20 may perform reconnection through the above-described wireless communication connection process, using a situation where the two devices are located within the certain distance, as a trigger.

The program and command storage unit 1011 receives a basic infusion program P from the controller 20 and stores it. However, the disclosure is not limited thereto, the program and command storage unit 1011 may receive and store an adjusted infusion command CP or an independent infusion command B from the controller 20. The basic infusion program P includes information about a basic infusion rate and pattern of a medical liquid for each time period, a maximum basic infusion rate, a target blood glucose range for each time period, and the like.

The infusion command generation unit 1012 includes a division algorithm and calculates a unit infusion amount Q based on the basic infusion program P stored in the program and command storage unit 1011. The unit infusion amount Q refers to an amount of medical liquid to be infused into a subject at every unit time. The unit time may be a preset time interval of several minutes, and may be, for example, 1 minute, 2 minutes, 5 minutes, 10 minutes, 15 minutes, or the like. Based on the basic infusion rate and pattern of the medical liquid for each time period as included in the basic infusion program P, The infusion command generation unit 1012 may calculate the unit infusion amount Q for the corresponding time period by using the division algorithm. For example, when a segment of the basic infusion program P is set such that the basic infusion rate from 12:00 AM to 6:00 AM is 0.50 U/hr, about 0.041667 U of medical liquid needs to be infused at 5-minute intervals during the time period of the segment, and the unit infusion amount Q is set to 0.041667 U based on a 5-minute interval.

The infusion command generation unit 1012 includes a conversion algorithm, and converts the calculated unit infusion amount Q into an electrical infusion signal S, and transmits the electrical infusion signal S to the medical liquid infusion control unit 1015 at every unit time. Here, the infusion signal S is a signal that generates a driving force in the driving module 300, herein, the driving force may be determined by the number of actuations of a pump within the driving module 300. For example, depending on the driving module 300, a planned medical liquid infusion amount per pump actuation may be set to 0.001 U, 0.005 U, 0.01 U, 0.05 U, or the like. Thus, the infusion command generation unit 1012 generates the infusion signal S by using a conversion algorithm based on the planned medical liquid infusion amount, and transmits the infusion signal S to the medical liquid infusion control unit 1015 at every unit time. The infusion signal S may be expressed as a current or a voltage.

The medical liquid infusion control unit 1015, based on the infusion signal S received from the infusion command generation unit 1012 at every unit time, controls the driving module 300 such that the medical liquid stored in the storage unit 200 is discharged through the needle N. Because the distance that the plunger 230 is moved by the driving unit 400 per single pump actuation is predetermined, a volume of medical liquid corresponding to this distance of the plunger 230 is drawn out through the needle N.

The medical liquid infusion control unit 1015 generates raw infusion information R after medical liquid infusion is completed at every unit time. The raw infusion information R may be an electrical signal received from the driving module 300, and includes the number of pump actuations actually performed as a result of infusion of the medical liquid. However, the raw infusion information R is not limited thereto and may further include an amount of change of the medical liquid stored in the storage unit 200, a moving distance of the plunger 230, and the like.

The infusion information calculation unit 1021 receives the raw infusion information R from the medical liquid infusion control unit 1015 at every unit time, and generates unit infusion information V based on the raw infusion information R. The unit infusion information V may include an actual unit infusion amount, which is the amount of medical liquid actually infused during the corresponding unit time. The infusion information calculation unit 1021 may include a calculation algorithm, and may calculate the actual unit medical liquid infusion amount during the corresponding unit time by using the number of pump actuations actually performed, which is included in the raw infusion information R, and the amount of medical liquid drawn out per pump actuation. However, the disclosure is not limited thereto, the infusion information calculation unit 1021 may calculate the actual unit medical liquid infusion amount based on an amount of change of the medical liquid stored in the storage unit, a moving distance of the plunger, or the like, in addition to the number of pump actuations.

After the actual medical liquid infusion is completed at every unit time, the infusion information storage unit 1022 receives and stores the unit infusion information V from the infusion information calculation unit 1021. When a wireless communication connection has been connected, the unit infusion information V stored in the infusion information storage unit 1022 is transmitted to the controller 20 through the wireless communication unit 1001, either immediately after being stored or at every preset time. Here, the preset time may be a preset time interval of several minutes, and may be, for example, 1 minute, 2 minutes, 5 minutes, 10 minutes, 15 minutes, or the like. In addition, the preset time may be equal to the unit time described above. However, when the wireless communication connection is released, the infusion information storage unit 1022 does not transmit the unit infusion information V to the controller 20, but accumulates the unit infusion information V in chronological order to generate cumulative infusion information W. The cumulative infusion information W may include, in chronological order, all unit infusion information V that could not be transmitted to the controller 20, from the time point when the wireless communication connection was released until the current time point before the wireless communication connection is reestablished.

The integrated control unit 1030 controls each constituent component of the medical liquid infusion device 10 based on various signals. For example, the integrated control unit 1030 may control the on/off state and the like of the alarm unit 800 by using a signal that controls the alarm unit 800, receive, from a sensor unit (not shown), signals corresponding to measurements of whether the device has been exposed to high temperatures for a long time, whether an injection port is occluded, whether there is medical liquid leakage, a device abnormality, a remaining battery capacity, and the like, as well as signals corresponding to measurements of the user's blood glucose level, blood pressure, heart rate, and the like. The integrated control unit 1030 may generate device data and biometric values based on these signals.

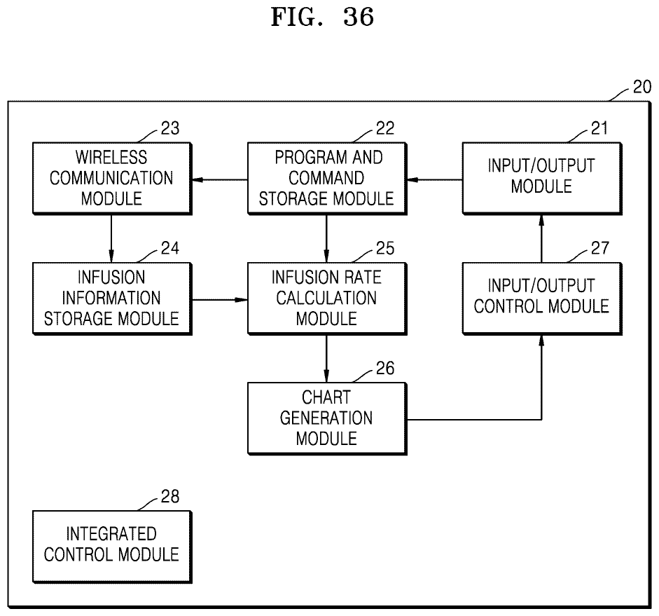

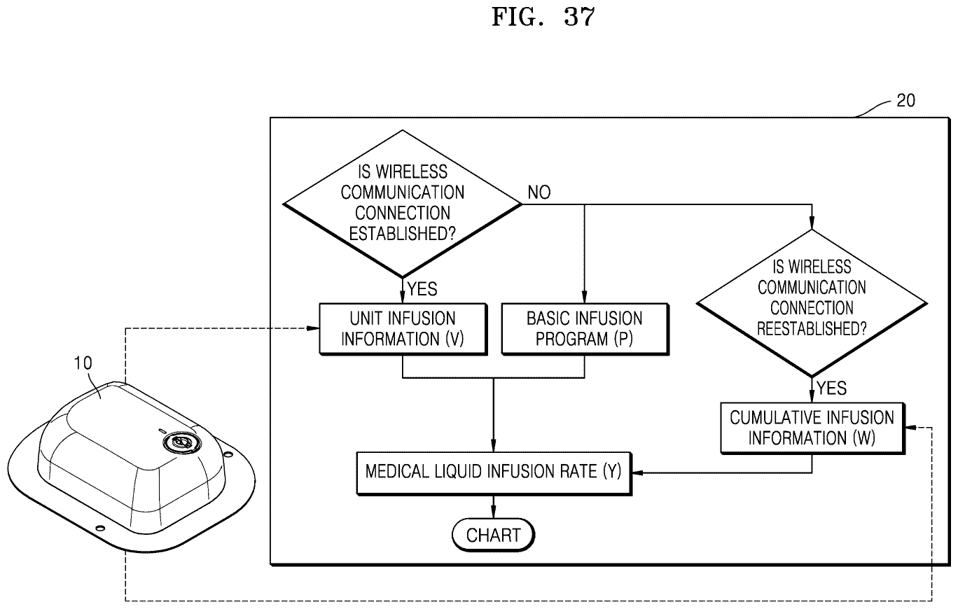

FIG. 36 is a block diagram schematically illustrating constituent components included in the controller 20 that are associated with an embodiment of the disclosure. FIG. 37 is a conceptual diagram schematically illustrating a flow of signals, data, and information that are received, transmitted, and generated by constituent components of the controller 20 of FIG. 36.

The controller 20 may include an input/output module 21, a program and command storage module 22, a wireless communication module 23, an infusion information storage module 24, an infusion rate calculation module 25, a chart generation module 26, an input/output control module 27, and an integrated control module 28. Each constituent component included in the controller 20 may be operated by a processing device designated as the integrated control module 28, such as a processor.

The input/output module 21 includes an input module and an output module, and may include an input module for receiving information from the user via a keyboard, a keypad, a virtual keyboard, a touch display, buttons, a camera, or the like, and an output module for outputting information to the user via a display, a speaker, light, a vibrator, or the like. For example, the input/output module 21 may be implemented as a touch display.

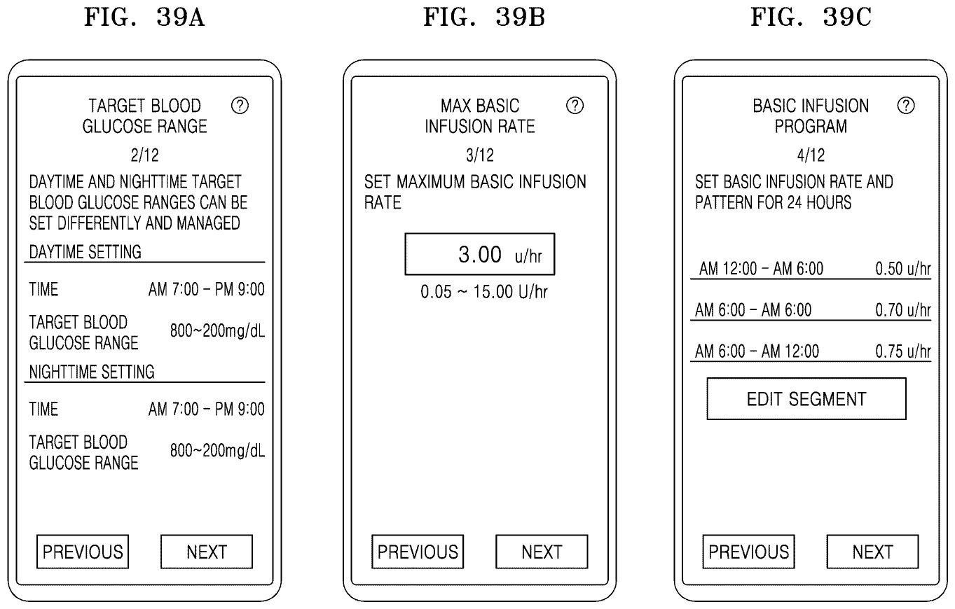

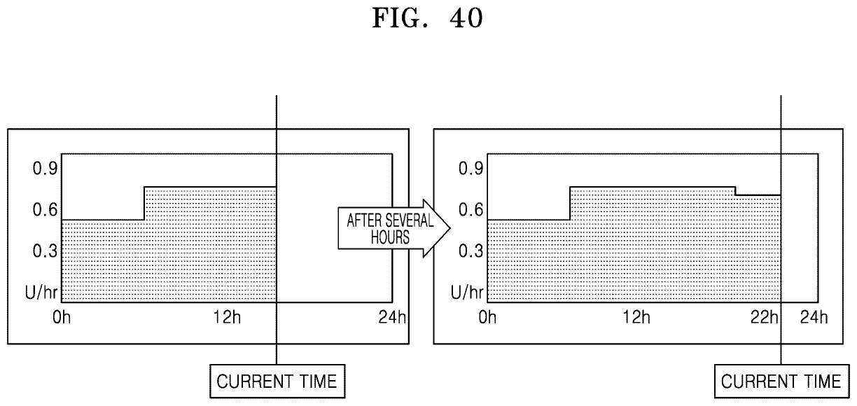

The input module included in the input/output module 21 receives the basic infusion program P from the user. A screen for receiving the basic infusion program will be described in detail with reference to FIGS. 39A to 39C. The output module displays, to the user, a chart generated by the chart generation module 26. A screens displaying the chart will be described in detail with reference to FIGS. 40, 42, and 44.

The program and command storage module 22 stores the basic infusion program P received from the user through the input/output module 21. However, the disclosure is not limited thereto, the program and command storage unit 22 may receive and store the adjusted infusion command CP or the independent infusion command B. The basic infusion program P may include information about a basic infusion rate and pattern of a medical liquid for each time period, a maximum basic infusion rate, a target blood glucose range for each time period, and the like.

The wireless communication module 23 establishes a connection for wireless communication enabling the controller 20 and the medical liquid infusion device 10 to transmit and receive signals. The wireless communication module 23 transmits a connection request signal and completes the wireless communication connection by transmitting and receiving connection parameters when the wireless connection starts. For example, similar to the wireless communication unit, the wireless communication module 23 may be a Bluetooth or Bluetooth Low Energy module.

While the wireless communication connection is maintained, the controller 20 may receive unit infusion information V from the medical liquid infusion device 10 at every unit time. However, when the wireless communication connection is released, the controller 20 cannot receive the unit infusion information V from the medical liquid infusion device 10. However, when the wireless communication connection is reestablished, the controller 20 may receive the unit infusion information V at every unit time from the point of reconnection onward, and may also receive cumulative infusion information W that includes the unit infusion information V accumulated during the period when wireless communication was disconnected.

The infusion information storage module 24 stores the unit infusion information V received from the medical liquid infusion device 10 at every unit time. The unit infusion information V may include an actual unit infusion amount, which is the amount of medical liquid actually infused during the corresponding unit time. In addition, the infusion information storage module 24 may store the cumulative medical liquid information W received from the medical liquid infusion device 10 after the wireless communication connection is reestablished. The cumulative infusion information W may include, in chronological order, all unit infusion information V that could not be transmitted to the controller 20, from the time point when the wireless communication connection was released until the wireless communication connection is reestablished.

The infusion rate calculation module 25 calculates a medical liquid infusion rate Y of the medical liquid infused from an initial time to the current time, based on the stored unit infusion information V. The infusion rate calculation module 25 may include a derivation algorithm, and may calculate, via the derivation algorithm, the medical liquid infusion rate Y by using the amount of medical liquid actually infused, which is included in the unit infusion information V, and a preset time or a unit time, which serves as a time interval at which the unit infusion information V is transmitted. In addition, the infusion rate calculation module 25 may also infer a medical liquid infusion rate corresponding to the current time, from the most recently received unit infusion information V. Because the interval of the preset time is significantly short, on the order of several minutes, the medical liquid infusion rate Y calculated based on the unit infusion information V most recently transmitted and closest to the current time may be inferred as the medical liquid infusion rate corresponding to the current time.

In addition, the infusion rate calculation module 25 may estimate the medical liquid infusion rate Y during a period when the wireless communication connection was released, by using the basic infusion rate and pattern of the medical liquid for each time period, which are stored in the basic infusion program P.

In addition, the infusion rate calculation module 25, based on the stored cumulative infusion information W, may calculate the medical liquid infusion rate Y for the medical liquid infused during the period from the time point when the wireless communication connection was released until the wireless communication connection is reestablished.

The chart generation module 26 generates a chart of the calculated medical liquid infusion rate Y over time. Here, the chart may be of any kind that may visually display the medical liquid infusion rate over time, such as a line graph, a bar graph, a point graph, an area graph, a table, or a combination thereof.

While the wireless communication connection is available, the chart generation module 26 may generate a first graph of the medical liquid infusion rate Y for the period from the time point when the medical liquid infusion device 10 was first used, until the current time point, by using the unit infusion information V received at every preset time.

In addition, for the period from the time point when the wireless communication connection was released until the current time point before the wireless communication connection is reestablished, the chart generation module 26 may generate a second graph of the estimated medical liquid infusion rate for the period from the time point when the wireless communication connection was released until the current time point, based on the basic infusion rate and pattern of the medical liquid for each time period, which are included in the basic infusion program P stored in the program and command storage module 22. Here, when the first graph previously exists, the second graph may be generated continuing from the last part of the first graph.

In addition, from the time point when the wireless communication connection is reestablished, the chart generation module 26 may again generate a first graph of the medical liquid infusion rate Y for the period from when the time point when the wireless communication connection is reestablished until the current time point, by using the unit infusion information V once again received at every preset time. In addition, the chart generation module 26 may update the second graph, which was generated at the time point when the wireless communication connection was disconnected, to a third graph, by using the medical liquid infusion rate Y calculated from the cumulative infusion information W received after the wireless communication connection was reestablished. Here, the first graph, which is generated after the wireless communication connection is reestablished as described above, may be generated continuing from the last part of the third graph.

Here, the first graph, the second graph, and the third graph may all be line graphs, the first graph and the third graph may be solid line graphs whose values are indicated by solid lines, and the second graph may be a dotted line graph whose values are indicated by dotted lines. However, the method of displaying the graphs is not limited thereto, and the type, form, legend, and the like of the graphs may vary.

The input/output control module 27 controls the input/output module 21 to display, to the user, a chart generated by the chart generation module 26. The input/output control module 27 may display the chart on the input/output module 21 in response to the user's request.

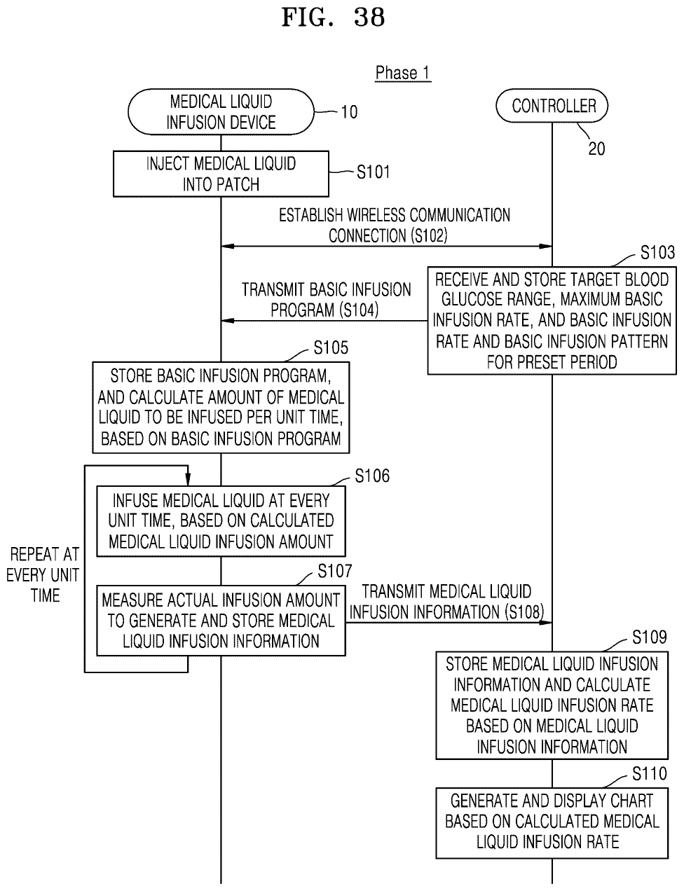

Hereinafter, a method of displaying medical liquid infusion, according to an embodiment of the disclosure, will be described with reference to FIGS. 38 to 44. In describing FIGS. 38 to 44, when referring to components identical to those described above, the previously used reference numerals will be used.

FIG. 38 is a flowchart illustrating a method of displaying medical liquid infusion in a first period Phase 1, according to an embodiment of the disclosure. FIGS. 39A to 39C illustrate screens of the controller 20 for explaining a medical liquid infusion method according to the basic infusion program P. FIG. 40 illustrates a medical liquid infusion information chart displayed on a screen of the controller 20 in the first period Phase 1. Here, the first period Phase 1 refers to a period after the medical liquid infusion device 10 and the controller 20 have initiated a wireless communication connection, during which the wireless communication connection is maintained without interruption.

In operation 101, a medical liquid is injected into the storage unit 200 of the medical liquid infusion device 10. The user may insert a syringe into a medical liquid injection port of an attachment portion of the medical liquid infusion device 10 to fill the storage unit 200 of the medical liquid infusion device 10 with a medical liquid.

In operation 102, a wireless communication connection is established between the medical liquid infusion device 10 and the controller 20. In detail, the medical liquid infusion device 10, upon a medical liquid being injected, i.e., triggered by the medical liquid injection, performs advertising to transmit an advertising message corresponding to a pairing request signal. In response to a new device registration request from a user desiring to register a new medical liquid infusion device 10, the controller 20 performs scanning for nearby medical liquid infusion devices 10, and transmits a connection request signal to the medical liquid infusion device 10. When the medical liquid infusion device 10 receives the connection request signal from the controller 20, a wireless connection starts, and the two devices complete the wireless communication connection by transmitting and receiving connection parameters. As described above, the wireless communication connection may be a Bluetooth or Bluetooth Low Energy connection.

In operation 103, the controller 20 receives and stores a basic infusion program P from the user. The basic infusion program P is an infusion method that schedules in advance for a medical liquid to be infused at a user-set rate, by dividing a defined period (e.g., 24 hours, 36 hours, or 48 hours) into segments. The basic infusion program P may be set by using the controller 20 that interworks with the medical liquid infusion device 10, or by using a platform provided by the integrated management server 30, such as a web or an application.

The basic infusion program P includes information about a basic infusion rate and pattern of a medical liquid for each time period, a maximum basic infusion rate, a target blood glucose range for each time period, and the like.

Referring to FIG. 39A, the user may set a target blood glucose range through the input/output module 21 of the controller 20. That is, daytime and nighttime periods may be defined, and then a target blood glucose range to be achieved in each time period may be set. Because blood glucose levels are likely to remain high during the day due to food consumption, a target blood glucose range different from that for the nighttime may be set to lower blood glucose accordingly and maintain it within a certain range. Referring to FIG. 39B, the user may set a maximum basic infusion rate of the medical liquid through the input/output module 21 of the controller 20, and referring to FIG. 39C, the user may set different basic infusion rates for different time periods by setting segments through the input/output module 21 of the controller 20. Because blood glucose may rise rapidly after food consumption, setting different basic infusion rates for different time periods in conjunction with meal or snack times helps to keep the blood glucose level within a normal blood glucose range.

In operation 104, the controller 20 transmits the set basic infusion program P to the medical liquid infusion device 10. Once set by the user, the basic infusion program P is transmitted one time through wireless communication. Based on the transmitted basic infusion program P, the medical liquid infusion device 10 infuses the medical liquid into a subject for a defined period (e.g., 24 hours, 36 hours, or 48 hours).

In operation 105, the medical liquid infusion device 10 stores the received basic infusion program P. In addition, based on the basic infusion program P, the medical liquid infusion device 10 calculates an amount of medical liquid to be infused per unit time. The unit infusion amount Q may be defined as the amount of medical liquid to be infused into a subject at every unit time. Here, when the medical liquid is insulin, the unit for the amount of medical liquid is U, which signifies unit, and 1 unit is 1/24 mg of purified insulin, and 1 mg of insulin may signify 24 units. In addition, here, the unit time may be a preset time interval of several minutes, and may be, for example, 1 minute, 2 minutes, 5 minutes, 10 minutes, 15 minutes, or the like. In detail, based on the basic infusion rate and pattern of the medical liquid for each time period as included in the basic infusion program P, the medical liquid infusion device 10 may calculate the unit infusion amount Q for the corresponding time period by using a division algorithm.

In operation 106, the medical liquid infusion device 10 infuses the calculated medical liquid infusion amount into the subject at every unit time. The calculated unit infusion amount Q may be converted into an electrical infusion signal S, the infusion signal S may generate a driving force in the driving module 300 to control the number of pump actuations, the pump may transmit the driving force to the driving unit 400 to move the plunger 230, and the medical liquid in the storage unit 200 may be discharged by the plunger 230 through the needle N to be infused into the subject.

In operation 107, when the medical liquid is infused, the medical liquid infusion device 10 measures a medical liquid infusion amount to generate medical liquid infusion information, and stores the medical liquid infusion information. When the medical liquid infusion is completed at every unit time, the medical liquid infusion device 10 generates raw infusion information R including the number of pump actuations actually performed, and based on the raw infusion information R, generates and stores medical liquid infusion information that includes the amount of medical liquid actually infused during the corresponding unit time. However, the disclosure is not limited thereto, and the raw infusion information R may further include an amount of change of the medical liquid stored in the storage unit or a moving distance of the plunger, and the medical liquid infusion information may then be generated based on this information.

In operation 108, the medical liquid infusion device 10 transmits the generated medical liquid infusion information to the controller 20. Operations 106 to 108 may be repeatedly performed at every unit time. For example, the medical liquid infusion device 10 infuses the calculated medical liquid infusion amount every 5 minutes based on the basic infusion program P, and after the infusion is completed, transmits a result, which is the actual infusion amount, to the controller 20 as medical liquid infusion information. Thus, after the medical liquid infusion is completed every 5 minutes, the controller 20 receives the medical liquid infusion information from the medical liquid infusion device 10.

In operation 109, the controller 20 stores the received medical liquid infusion information and, based on the stored medical liquid infusion information, calculates a medical liquid infusion rate Y. The medical liquid infusion information may include an actual unit infusion amount, which is the amount of medical liquid actually infused during the corresponding unit time. The controller 20 may calculate the medical liquid infusion rate Y by using the amount of medical liquid actually infused and a preset time or a unit time, which is a time interval at which the medical liquid infusion information is transmitted.

In operation 110, the controller 20 generates and displays a chart of the medical liquid infusion rate over time, based on the calculated medical liquid infusion rate Y. For example, the chart may be a line graph. In addition, a line graph generated in the first period Phase 1 may be indicated by, for example, a solid line. Referring to FIG. 40, the vertical axis represents the medical liquid infusion rate (U/hr), where U denotes a unit, and 1 unit is 1/24 mg of purified insulin, and 1 mg of insulin signifies 24 units. The horizontal axis represents time (hour). The solid line graph is generated based on the calculated medical liquid infusion rate, and the shaded area in a lower graph may correspond to the amount of medical liquid administered during the corresponding time, that is, the amount of insulin (U). The controller 20 continuously receives medical liquid infusion information from the medical liquid infusion device 10 through wireless communication at every preset time. Thus, a continuously connected solid line graph after several hours may be confirmed, as in the left graph changing to the right graph.

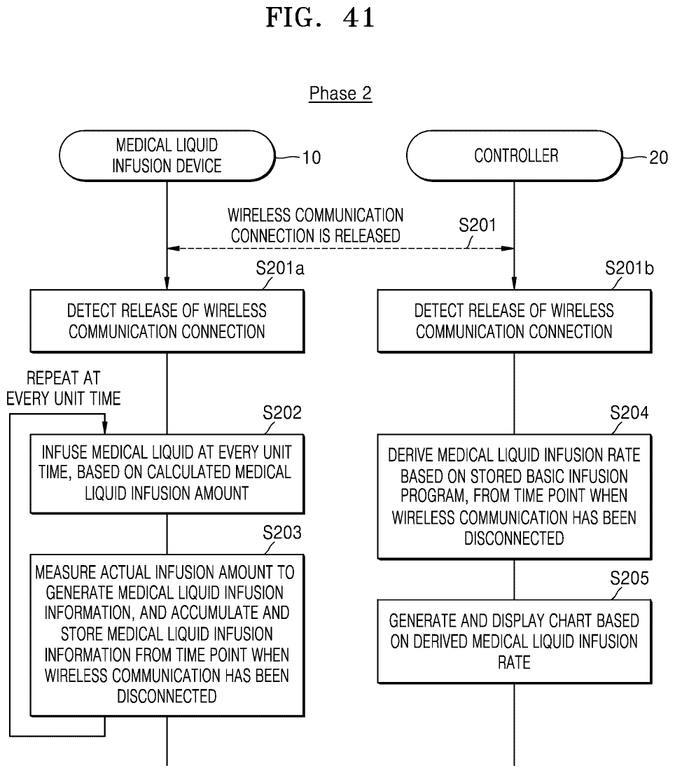

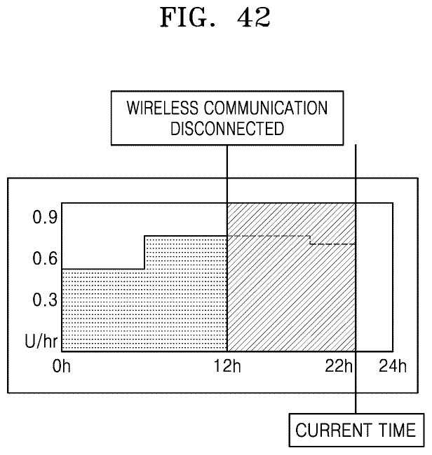

FIG. 41 is a flowchart illustrating a method of displaying medical liquid infusion in a second period Phase 2, according to an embodiment of the disclosure. FIG. 42 illustrates a medical liquid infusion information chart displayed on the controller 20 in the second period Phase 2. Here, the second period Phase 2 refers to a period when an event has occurred in the wireless communication connection between the medical liquid infusion device 10 and the controller 20. In other words, it refers to a period when the wireless communication connection between the medical liquid infusion device 10 and the controller 20 is released or disconnected.

In operation 201, a wireless communication-related event occurs. Here, the event may be a release or disconnection of the wireless communication connection. For example, when the distance between the controller 20 and the medical liquid infusion device 10 exceeds a threshold distance, the wireless communication connection may be automatically released. In operations 201a and 201b, the medical liquid infusion device 10 and the controller 20 may detect the release of the wireless communication connection.

In operation 202, as in operation 106, the medical liquid infusion device 10 infuses the calculated medical liquid infusion amount into the subject at every unit time. The calculated unit infusion amount Q may be converted into an electrical infusion signal S, the infusion signal S may generate a driving force in the driving module 300 to control the number of pump actuations, the pump may transmit the driving force to the driving unit 400 to move the plunger 230, and the medical liquid in the storage unit 200 may be infused into the subject by the plunger 230 through the needle N.

In operation 203, similarly to operation 107, when the medical liquid is infused, the medical liquid infusion device 10 measures a medical liquid infusion amount to generate medical liquid infusion information, and stores the medical liquid infusion information. When the medical liquid infusion is completed at every unit time, the medical liquid infusion device 10 generates raw infusion information including the number of pump actuations actually performed, and based on the raw infusion information, generates and stores medical liquid infusion information that includes the amount of medical liquid actually infused during the corresponding unit time. However, the disclosure is not limited thereto, and the raw infusion information may further include an amount of change of the medical liquid stored in the storage unit 200 or a moving distance of the plunger 230, and the medical liquid infusion information may then be generated based on this information.

The difference between operation 203 and operation 107 is that, in operation 203, the medical liquid infusion device 10 accumulates and stores, in chronological order, the medical liquid infusion information generated from the time point when the wireless communication event has occurred. That is, when the medical liquid infusion device 10 in the second period Phase 2 detects a wireless communication connection-related event, then unlike in operation 108 for the first period Phase 1, the medical liquid infusion device 10 does not transmit the generated medical liquid infusion information to the controller 20.

Operations 202 and 203 may be repeatedly performed at every unit time. For example, the medical liquid infusion device 10 infuses the calculated medical liquid infusion amount every 5 minutes based on the basic infusion program P, and after the infusion is completed, accumulates and stores results, which are actual infusion amounts, in chronological order.

In operation 204, after detecting the wireless communication-related event, the controller 20 derives a medical liquid infusion rate Y based on the stored basic infusion program P, from the time point when the wireless communication has been disconnected. The basic infusion program P includes the stored basic infusion rate and pattern of the medical liquid for each time period. Thus, even when the controller 20 does not receive medical liquid infusion information from the medical liquid infusion device 10 during the period the wireless communication connection is released, the controller 20 may estimate the medical liquid infusion rate Y based on the stored basic infusion program P.

In operation 205, the controller 20 generates and displays a chart of the medical liquid infusion rate over time, based on the derived medical liquid infusion rate Y. For example, the chart may be a line graph. In addition, a line graph generated in the second period Phase 2 may be displayed to be distinguishable from the graph generated in the first period Phase 1, and may be indicated by, for example, a dotted line. Referring to FIG. 42, the vertical axis represents the medical liquid infusion rate (U/hr), where U denotes a unit, and 1 unit is 1/24 mg of purified insulin, and 1 mg of insulin signifies 24 units. The horizontal axis represents time (hour). The dotted line graph is generated based on the estimated medical liquid infusion rate. Assuming that the chart on the right in FIG. 40 and the chart in FIG. 42 represent the same time period (e.g., between 12 h and 22h), it may be confirmed that, in the chart of FIG. 42, unlike the chart of FIG. 40, the graph is indicated by a dotted line from the moment the wireless communication disconnection has occurred up to the current time.

Even when a wireless communication-related event occurs and medical liquid infusion information that the controller 20 has received every hour from the medical liquid infusion device 10 is interrupted, the controller 20 may still provide the user with information related to medical liquid infusion from the time of the communication interruption to the current time. Furthermore, the information related to medical liquid infusion during the second period Phase 2 is derived based on information different from that of the graph during the first period Phase 1, and thus may be displayed to be distinguishable from the graph of the first period Phase 1.

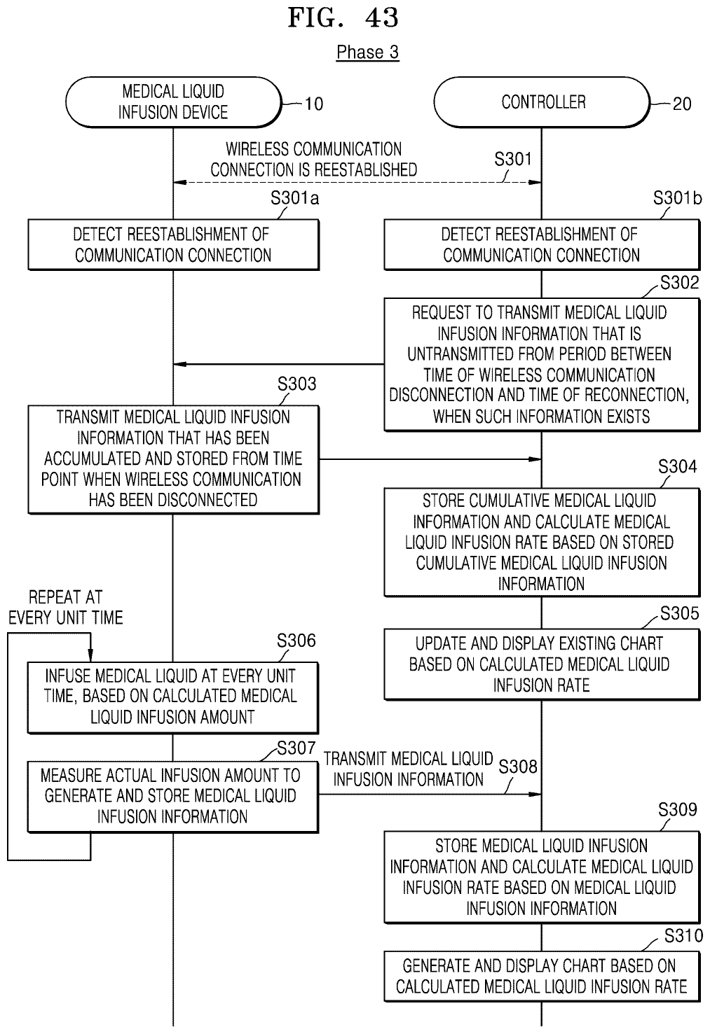

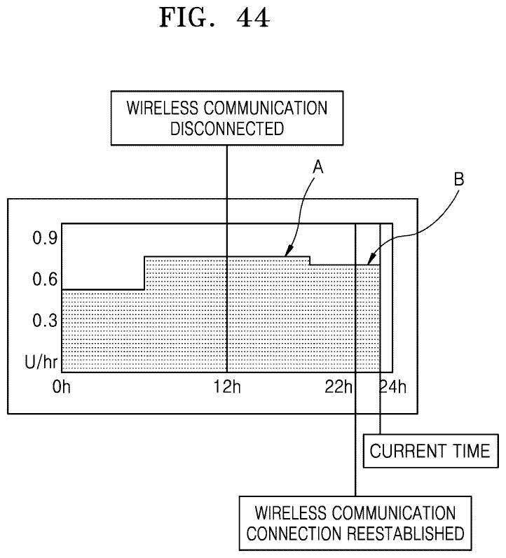

FIG. 43 is a flowchart illustrating a method of displaying medical liquid infusion in a third period Phase 3, according to an embodiment of the disclosure. FIG. 44 illustrates a medical liquid infusion information chart displayed on the controller 20 in the third period Phase 3. Here, the third period Phase 3 refers to a period when an event that had occurred in the wireless communication connection between the medical liquid infusion device 10 and the controller 20 has been resolved. In other words, it refers to a period when the time point when the wireless communication connection between the medical liquid infusion device 10 and the controller 20 is reestablished.

In operation 301, the event that has occurred in relation to wireless communication is resolved. For example, when the distance between the controller 20 and the medical liquid infusion device 10 comes within the threshold distance, the wireless communication connection may be automatically reestablished. In addition, the user may discover the release of the wireless communication connection between the controller 20 and the medical liquid infusion device 10, and reestablish the wireless communication connection. In operations 301a and 301b, the medical liquid infusion device 10 and the controller 20 may detect the release of the wireless communication connection.

When the reestablishment of the wireless communication connection is detected, the medical liquid infusion device 10 transmits, to the controller 20, cumulative medical liquid infusion information, which has been accumulated and stored from the time point when the wireless communication has been disconnected.

According to an embodiment, as in operation 302, the cumulative medical liquid infusion information may be transmitted in response to a request from the controller 20. In detail, when the controller 20 detects the reestablishment of the wireless communication connection, the controller 20 may request the medical liquid infusion device 10 to transmit any medical liquid infusion information that is untransmitted from the period between the time of wireless communication disconnection and the time of reconnection, when such information exists. In this case, as in operation 303, the medical liquid infusion device 10 may transmit the medical liquid infusion information that has been accumulated and stored from the time point when the wireless communication has been disconnected.

According to another embodiment, as in operation 303, the medical liquid infusion device 10, upon detecting the reestablishment of the wireless communication connection, may automatically transmit the cumulative medical liquid infusion information to the controller 20. In this case, even without a request such as that in operation 302, the medical liquid infusion device 10 may transmit the medical liquid infusion information that has been accumulated and stored from the time point when the wireless communication has been disconnected.

In operation 304, the controller 20 stores the received cumulative medical liquid information and calculates a medical liquid infusion rate Y based on the stored cumulative medical liquid infusion information. The cumulative medical liquid infusion information may include, in chronological order, the amounts of medical liquid actually infused at every unit time that could not be transmitted to the controller 20, from the time point when the wireless communication connection has been released until the wireless communication connection is reestablished. The controller 20 may calculate the medical liquid infusion rate Y by using the amounts of medical liquid actually infused, the unit time, and the period of wireless communication disconnection.

In operation 305, the controller 20 may update the chart generated in operation 205 based on the calculated medical liquid infusion rate. Here, the chart generated in operation 205 refers to a chart generated based on the medical liquid infusion rate that has been estimated or derived from the basic infusion program P during the period of communication disconnection. For example, the chart updated in operation 305 may be a line graph. A line graph generated in the third period Phase 3 may be indicated by a solid line, similar to the line graph generated in the first period Phase 1. In other words, the graph generated in the third period is distinguishable from that generated in the second period, but may be represented in the same manner as that of the first period.

The vertical axis of FIG. 44 represents the medical liquid infusion rate (U/hr), where U denotes a unit, and 1 unit is 1/24 mg of purified insulin, and 1 mg of insulin signifies 24 units. The horizontal axis represents time (hour). It is assumed that the chart of FIG. 44 further includes an additional time period (e.g., part B, from 22 h to 24 h) compared to the chart of FIG. 42. It may be confirmed that the part indicated as ‘A’ in FIG. 44, which was previously the part indicated by the dotted line graph in FIG. 42, has been updated by operation 305 to be a solid line. Because the part indicated as ‘B’ in FIG. 44 is the portion after the wireless communication connection has been reestablished, it may be confirmed that this part is indicated by a solid line, similar to FIG. 40. In this manner, after a wireless communication-related event has been resolved, the controller 20 may receive accumulated medical liquid infusion information from the medical liquid infusion device 10 and, based on this information, update the information that was provided to the user during the period of communication disconnection.

In addition, operations 304 and 305 may be performed before operation 309, concurrently with operation 310, or after operation 310.

In operation 306, as in operation 106, the medical liquid infusion device 10 infuses the calculated medical liquid infusion amount into the subject at every unit time. In operation 307, as in operation 107, when the medical liquid is infused, the medical liquid infusion device 10 measures a medical liquid infusion amount to generate medical liquid infusion information, and stores the medical liquid infusion information. In addition, because the wireless communication connection has been reestablished, in operation 308, as in operation 108, the medical liquid infusion device 10 transmits the generated medical liquid infusion information to the controller 20. Operations 306 to 308 may be repeatedly performed at every unit time.

In operation 309, as in operation 109, the controller 20 stores the received medical liquid infusion information and, based on the stored medical liquid infusion information, calculates a medical liquid infusion rate Y. In operation 310, as in operation 110, the controller generates and displays a chart of the medical liquid infusion rate over time, based on the calculated medical liquid infusion rate Y. The line graph generated in operation 310, as shown in part B of FIG. 44, may be indicated by a solid line graph, similar to that generated in the first period.

As described above, according to an embodiment of the disclosure, provided is a method of displaying a medical liquid infusion result, which may provide reliability to a user because, even when a problem occurs in the wireless communication connection between a medical liquid infusion device and a controller, medical liquid infusion information may be estimated by using a basic infusion program stored in the controller and provided to the user even while wireless communication is disconnected.

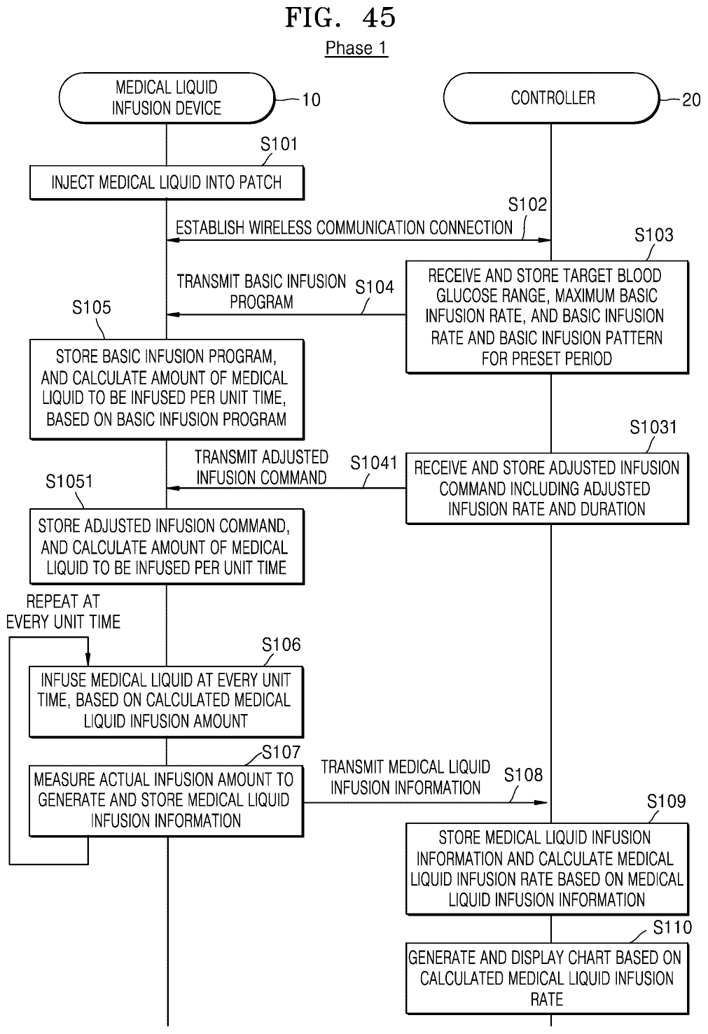

A method of displaying medical liquid infusion according to another embodiment of the disclosure is for a situation where an adjusted infusion command CP is added to a basic infusion program P. Hereinafter, a method of displaying medical liquid infusion according to another embodiment of the disclosure will be described with reference to FIGS. 45 to 50. In describing FIGS. 45 to 50, when referring to components identical to those described above, the previously used reference numerals will be used.

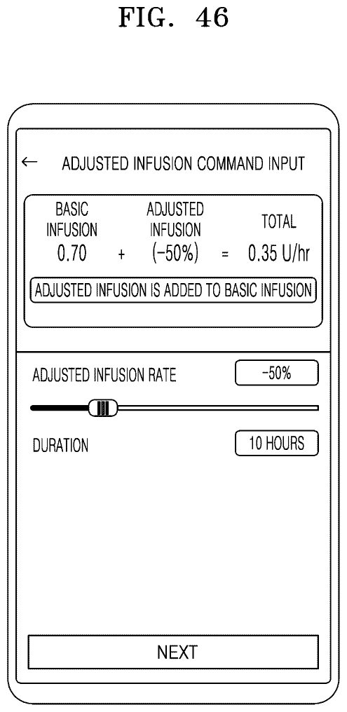

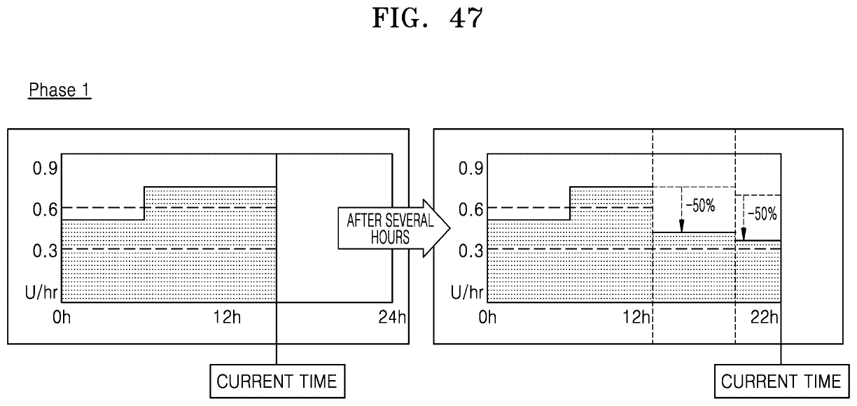

FIG. 45 is a flowchart illustrating a method of displaying medical liquid infusion in a first period Phase 1, according to another embodiment of the disclosure. FIG. 46 illustrates a screen of the controller 20 for explaining a medical liquid infusion method according to an adjusted infusion command CP. FIG. 47 illustrates a medical liquid infusion information chart displayed on a screen of the controller 20 in the first period Phase 1. Here, the first period Phase 1 refers to a period after the medical liquid infusion device 10 and the controller 20 have initiated a wireless communication connection, during which the wireless communication connection is maintained without interruption.

Operations 101 to 105 are identical to operations 101 to 105 of FIG. 38 described above, and thus, redundant descriptions thereof will be omitted.

In operation 1031, the controller 20 receives and stores the adjusted infusion command CP from the user. The basic infusion program P is an infusion method that schedules in advance for a medical liquid to be infused at a user-set rate by dividing a defined period (e.g., 24 hours, 36 hours, or 48 hours) into segments, whereas the adjusted infusion command CP refers to a command or program that temporarily changes the basic infusion rate of an already scheduled basic infusion program, and it is also referred to as temporary basic infusion. Similar to the basic infusion program P, the adjusted infusion command may be set by using the controller 20 that interworks with the medical liquid infusion device 10, or by using a platform provided by the integrated management server 30, such as a web or an application.

The basic infusion program P includes information about a basic infusion rate and pattern of a medical liquid for each time period, a maximum basic infusion rate, a target blood glucose range for each time period, and the like, whereas the adjusted infusion command CP includes information about an adjusted infusion rate, a duration, and the like. Here, the adjusted infusion rate refers to an increment or decrement value relative to the basic infusion rate, or a constant rate value set within a maximum basic infusion rate range. Accordingly, the adjusted infusion command CP may decrease or increase the preset basic infusion rate for a set duration. In addition, here, the duration may be adjusted within a set range (e.g., from a minimum of 30 minutes to a maximum of 12 hours).

Referring to FIG. 46, the user may set the adjusted infusion rate and the duration through the input/output module 21 of the controller 20. The adjusted infusion rate may be represented with units of increment or decrement as a percentage (%) or as a rate value (U/hr). As illustrated in FIG. 46, when the unit of increment or decrement for the adjusted infusion rate is percentage (%), the increment or decrement relative to the basic infusion rate preset by the basic infusion program P is adjusted in percentage points. The adjusted infusion rate may be selected within a range of −100% to 100%. When −100% is input as the adjusted infusion rate, the basic infusion of medical liquid, which is preset by the basic infusion program P, may be temporarily stopped. Although not illustrated, the adjusted infusion rate may also be input as a specific rate value (U/hr) selected from within the maximum basic infusion rate range (e.g., 0.03 U/hr, or 0.80 U/hr).

Operation 1031 may be performed at any time after operation 103 is set, and is not limited to the timing illustrated in FIG. 45.

In operation 1041, the controller 20 transmits the adjusted infusion command CP to the medical liquid infusion device 10. Once set by the user, the adjusted infusion command CP is transmitted one time through wireless communication. The medical liquid infusion device 10 reflects the transmitted adjusted infusion command CP in the basic infusion program P and then infuses the medical liquid into the subject at the adjusted infusion rate for the duration.

In operation 1051, the medical liquid infusion device 10 stores the received adjusted infusion command CP. In addition, based on the adjusted infusion command CP, the medical liquid infusion device 10 calculates an amount of medical liquid to be infused per unit time. When the adjusted infusion rate is input as a percentage, the medical liquid infusion device 10 may calculate the amount of medical liquid by reflecting the adjusted infusion command CP in the basic infusion program P, whereas, when the adjusted infusion rate is input as a rate value (U/hr), the medical liquid infusion device 10 may calculate the amount of medical liquid by considering only the adjusted infusion command CP. Here, the unit infusion amount Q may be defined as the amount of medical liquid to be infused into a subject at every unit time. In addition, here, the unit time may be a preset time interval of several minutes, and may be, for example, 1 minute, 2 minutes, 5 minutes, 10 minutes, 15 minutes, or the like. In detail, the medical liquid infusion device 10 may calculate the unit infusion amount Q for the corresponding time period by using a division algorithm, based on the adjusted infusion rate and the duration.

Next, the medical liquid infusion device 10 infuses the calculated medical liquid infusion amount into the subject at every unit time, and when the medical liquid infusion is completed at every unit time, generates raw infusion information R including the number of pump actuations actually performed, and based on the raw infusion information R, generates and stores medical liquid infusion information, which includes the amount of medical liquid actually infused during the corresponding unit time, and transmits the generated medical liquid infusion information to the controller 20. The controller 20 stores the received medical liquid infusion information, calculates a medical liquid infusion rate Y based on the stored medical liquid infusion information, and generates and displays a chart of the medical liquid infusion rate over time based on the calculated medical liquid infusion rate Y. Operations 106 to 109 are identical to operations 106 to 109 of FIG. 38 described above, and thus, redundant descriptions thereof will be omitted.

In operation 110, the controller 20 generates and displays a chart of the medical liquid infusion rate over time, based on the calculated medical liquid infusion rate Y. For example, the chart may be a line graph. In addition, a line graph generated in the first period Phase 1 may be indicated by, for example, a solid line. Referring to FIG. 47, the vertical axis represents the infusion rate (U/hr), and the horizontal axis represents time (hour). The solid line graph is generated based on the calculated medical liquid infusion rate, and the shaded area in a lower graph may correspond to the amount of medical liquid administered during the corresponding time, that is, the amount of insulin (U). The controller 20 continuously receives medical liquid infusion information from the medical liquid infusion device 10 through wireless communication at every preset time. Thus, a continuously connected solid line graph after several hours may be confirmed, as in the left graph changing to the right graph.

In relation to FIG. 47, assuming that the chart on the right in FIG. 47 and the chart on the right in FIG. 40 represent the same time period (e.g., between 12 h and 22 h), a comparison is made between the previous embodiment related to the basic infusion program P and the current embodiment related to the adjusted infusion command CP. The chart of FIG. 47, as illustrated in FIG. 46, is a medical liquid infusion information chart for the first period Phase in which the adjusted infusion command CP including an adjusted infusion rate of −50% and a duration of 10 hours (e.g., assumed to be from 12 h to 22 h) was input at the 12 h time point. Thus, compared to the chart on the right in FIG. 40, it may be confirmed that in the chart on the right in FIG. 47, an adjusted infusion rate representing a 50% decrease from the basic infusion rate is displayed from the 12 h time point, which is the start point of the duration, up to the current time of 22 h.

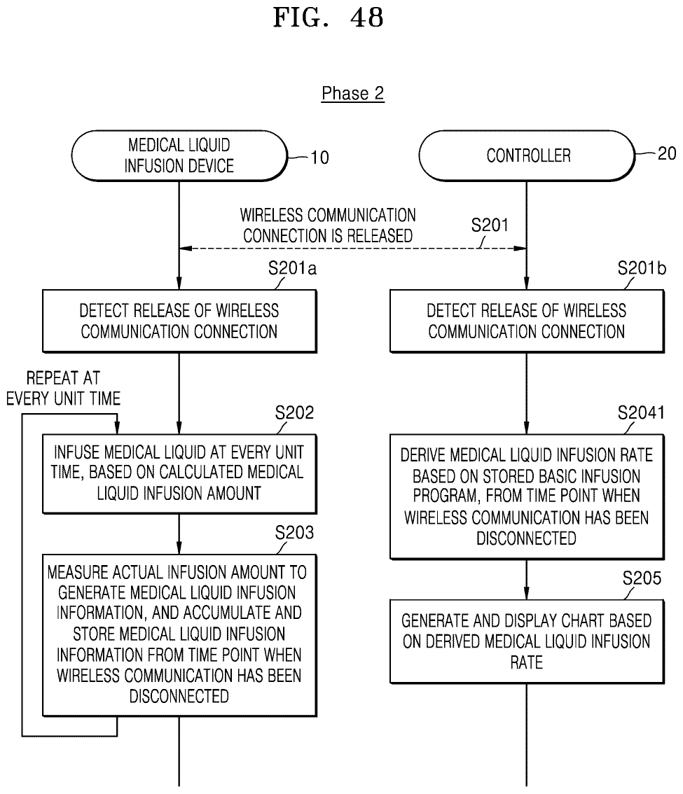

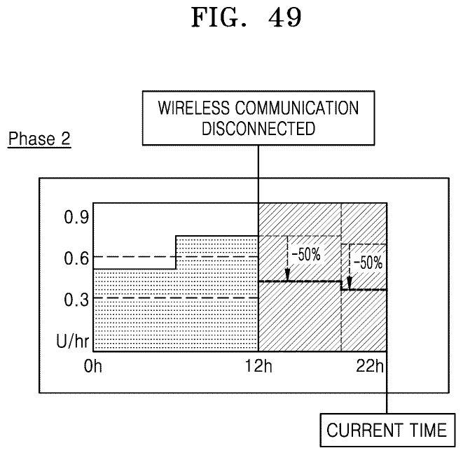

FIG. 48 is a flowchart illustrating a method of displaying medical liquid infusion in a second period Phase 2, according to another embodiment of the disclosure. FIG. 49 illustrates a medical liquid infusion information chart displayed on the controller 20 in the second period Phase 2. Here, the second period Phase 2 refers to a period when an event has occurred in the wireless communication connection between the medical liquid infusion device 10 and the controller 20. In other words, it refers to a period when the wireless communication connection between the medical liquid infusion device 10 and the controller 20 is released or disconnected.

Operations 201 to 201b are identical to operations 201 to 201b of FIG. 41 described above, and thus, redundant descriptions thereof will be omitted. In addition, operations 202 to 203 are also identical to operations 202 to 203 of FIG. 41 described above, and thus, redundant descriptions thereof will be omitted.

In operation 2041, after detecting the wireless communication-related event, the controller 20 derives a medical liquid infusion rate Y based on the stored adjusted infusion command CP, from the time point when the wireless communication has been disconnected. The adjusted infusion command CP includes an adjusted infusion rate of the medical liquid for a duration. Thus, even when the controller 20 does not receive medical liquid infusion information from the medical liquid infusion device 10 during the period the wireless communication connection is released, the controller 20 may estimate the medical liquid infusion rate Y based on the stored adjusted infusion command CP.

In operation 205, the controller 20 generates and displays a chart of the medical liquid infusion rate over time, based on the derived medical liquid infusion rate Y. For example, the chart may be a line graph. In addition, a line graph generated in the second period Phase 2 may be displayed to be distinguishable from the graph generated in the first period Phase 1, and may be indicated by, for example, a dotted line. Referring to FIG. 49, the vertical axis represents the infusion rate (U/hr), and the horizontal axis represents time (hour). The dotted line graph is generated based on the estimated medical liquid infusion rate.

Assuming that the chart on the right in FIG. 47 and the chart in FIG. 49 represent the same time period (e.g., between 12 h and 22 h), it may be confirmed that, in the chart of FIG. 49, unlike the chart of FIG. 47, the graph is indicated by a dotted line from the moment the wireless communication disconnection has occurred up to the current time. In addition, for the graph indicated by the dotted line, it may be confirmed that an adjusted infusion rate representing a 50% decrease from the basic infusion rate is displayed.

As such, even when a wireless communication-related event occurs and medical liquid infusion information that the controller 20 has received every hour from the medical liquid infusion device 10 is interrupted, the controller 20 may still provide the user with information related to medical liquid infusion from the time of the communication interruption to the current time. Furthermore, the information related to medical liquid infusion during the second period Phase 2 is derived based on information different from that of the graph during the first period Phase 1, and thus may be displayed to be distinguishable from the graph of the first period Phase 1.

The method of displaying medical liquid infusion for the third period Phase 3 is identical to the method of displaying medical liquid infusion illustrated in FIG. 43, redundant drawings and descriptions thereof will be omitted. Here, the third period Phase 3 refers to a period when an event that had occurred in the wireless communication connection between the medical liquid infusion device 10 and the controller 20 has been resolved. In other words, it refers to a period when the time point when the wireless communication connection between the medical liquid infusion device 10 and the controller 20 is reestablished.

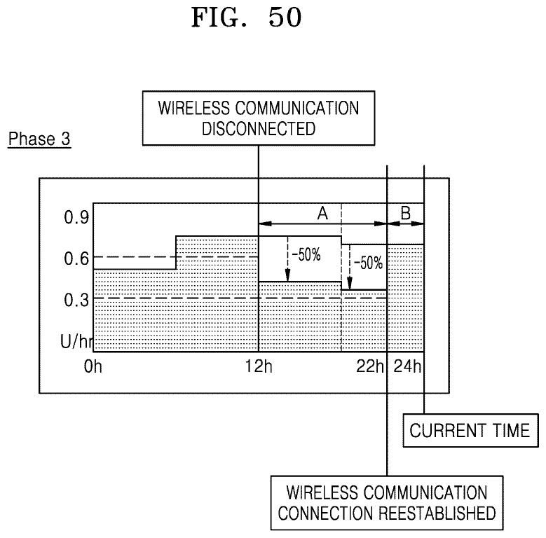

FIG. 50 illustrates a medical liquid infusion information chart displayed on the controller 20 in a third period Phase 3, according to another embodiment of the disclosure. When the wireless communication connection is reestablished, the controller 20 receives cumulative medical liquid infusion information and, by using a medical liquid infusion rate calculated based on the cumulative medical liquid infusion information, updates a chart that has been generated based on a medical liquid infusion rate estimated or derived from the adjusted infusion command CP during the period of communication disconnection. Thus, a line graph generated in the third period Phase 3 may be indicated by a solid line, similar to the line graph generated in the first period Phase 1. The vertical axis of FIG. 49 represents the infusion rate (U/hr), and the horizontal axis represents time (hour). It is assumed that the chart of FIG. 50 further includes an additional time period (e.g., part B, from 22 h to 24 h) compared to the chart of FIG. 49. It may be confirmed that the part indicated as ‘A’ in FIG. 50, which was previously the part indicated by the dotted line graph in FIG. 49, has been updated to be a solid line. Because the part indicated as ‘B’ in FIG. 50 is the portion after the wireless communication connection has been reestablished, it may be confirmed that this part is indicated by a solid line, similar to FIG. 47. In addition, it may be confirmed that, because the graph for the part indicated as ‘A’ in FIG. 50 corresponds to the duration set by the adjusted infusion command CP (e.g., assumed to be from 12 h to 22 h), the infusion rate is displayed as the adjusted infusion rate, which is a 50% decrease from the basic infusion rate, and because the graph for the part indicated as ‘B’ corresponds to the period after the duration, the infusion rate has reverted to the basic infusion rate. In this manner, after a wireless communication-related event has been resolved, the controller 20 may receive accumulated medical liquid infusion information from the medical liquid infusion device 10 and, based on this information, update the information that was provided to the user during the period of communication disconnection.

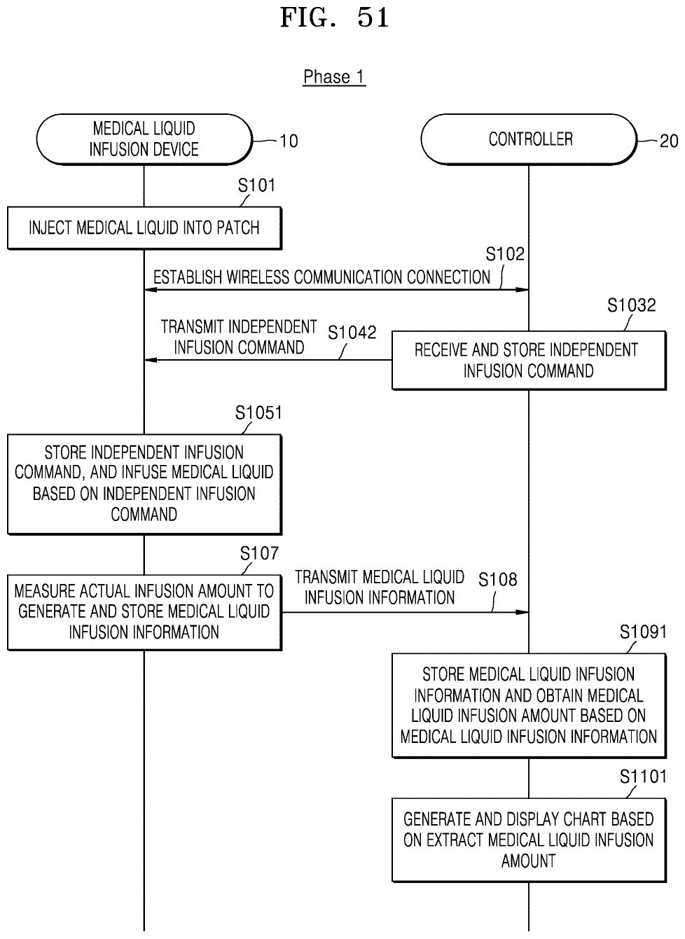

A method of displaying medical liquid infusion according to yet another embodiment of the disclosure is for a situation where an independent infusion command B, which is distinct from the basic infusion program P and the adjusted infusion command CP, is input. Hereinafter, a method of displaying medical liquid infusion according to yet another embodiment of the disclosure will be described with reference to FIGS. 51 to 57. In describing FIGS. 51 to 57, when referring to components identical to those described above, the previously used reference numerals will be used.

The independent infusion command B is a command to infuse a certain amount of medical liquid, and may include a first independent infusion command (also referred to as an immediate infusion command) for infusing a set amount of medical liquid immediately over a short period, and a second independent infusion command (also referred to as an extended infusion command) for infusing a set amount of medical liquid constantly over a set period. When the medical liquid is insulin, an independent infusion command may be used, for example, to lower blood glucose that rises suddenly due to food intake, or when it is necessary to correct a high blood glucose level in the body to fall within a normal blood glucose range.

The independent infusion command B is a command independent of the basic infusion program P and/or the adjusted infusion command CP, and may be used together with the basic infusion program P and/or the adjusted infusion command CP, and only the independent infusion command B may be used separately, and various combinations with other programs and commands are possible. Because descriptions of other programs and commands have already been provided above, for convenience of description, only a case in which the independent infusion command B is used alone will be described in detail hereinafter.

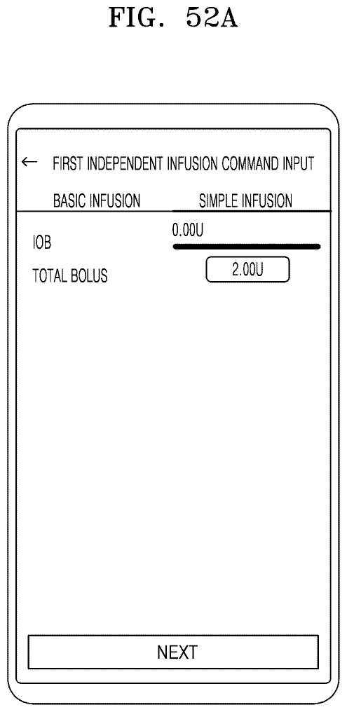

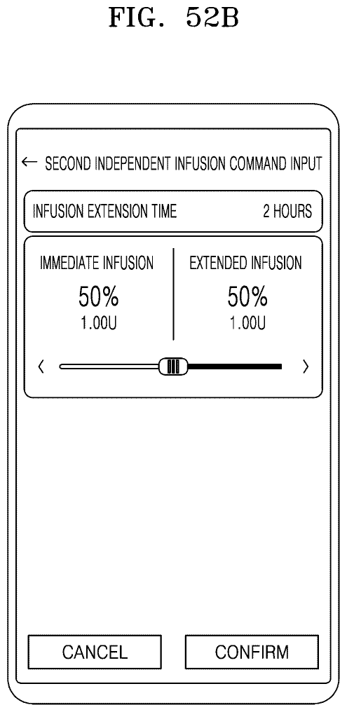

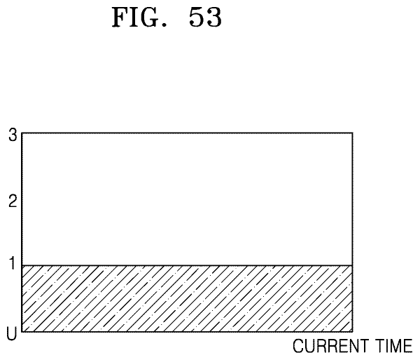

FIG. 51 is a flowchart illustrating a method of displaying medical liquid infusion in a first period Phase 1, according to yet another embodiment of the disclosure. FIG. 52 illustrates a screen of the controller 20 for explaining a medical liquid infusion method according to an independent infusion command B. FIG. 53 illustrates a medical liquid infusion information chart displayed on a screen of the controller 20 in the first period Phase 1. Here, the first period Phase 1 refers to a period after the medical liquid infusion device 10 and the controller 20 have initiated a wireless communication connection, during which the wireless communication connection is maintained without interruption.

Operations 101 and 102 are identical to operations 101 and 102 of FIG. 38 described above, and thus, redundant descriptions thereof will be omitted.

In operation 1032, the controller 20 receives an stores the independent infusion command B from the user. The independent infusion command B is a command for infusing a certain amount (units) of medical liquid, and may be set by using the controller 20 that interworks with the medical liquid infusion device 10, or by using a platform provided by the integrated management server 30, such as a web or an application.

The independent infusion command B includes information about an infusion amount. The infusion amount may be directly input by the user in units (e.g., in FIG. 52A, the infusion amount is 2.00 U), or may be a result calculated by a platform of the controller 20 or the integrated management server 30, based on receiving an input such as measurement information about biometric values (e.g., blood glucose measured by the medical liquid infusion device 10) and the amount of food (carbohydrates) to be consumed by the subject. The independent infusion command B may further include information about an infusion extension time in addition to the infusion amount, and in this case, the independent infusion command B may be referred to as a second independent infusion command. The infusion extension time refers to a time period for gradually infusing the infusion amount by dividing it constantly over an input extended infusion time (e.g., in FIG. 52B, the infusion extension time is 2 hours). When the medical liquid is insulin, setting the infusion extension time has the effect of enabling insulin to be infused into the subject in accordance with the digestion rate and blood glucose change trend observed when food that causes a slow change in blood glucose is consumed.

In operation 1042, the controller 20 transmits the independent infusion command B to the medical liquid infusion device 10. Once set by the user, the independent infusion command B is transmitted one time through wireless communication.

In operation 1051, the medical liquid infusion device 10 stores the received independent infusion command B. In addition, the medical liquid infusion device 10 infuses the medical liquid into the subject based on the infusion amount included in the independent infusion command B. In an optional embodiment, in the case of a second independent infusion command, the medical liquid infusion device 10 calculates an amount of unit medical liquid to be infused per unit time based on the infusion amount and the infusion extension time included in the command, and then infuses the medical liquid into the subject based on the calculated amount.

Next, when the medical liquid infusion is completed, the medical liquid infusion device 10 generates raw infusion information R including the number of pump actuations actually performed, and based on the raw infusion information R, generates and stores medical liquid infusion information that includes the amount of medical liquid actually infused, and transmits the generated medical liquid infusion information to the controller 20. In the case of a second independent infusion command, the manner in which the medical liquid is infused at every unit time differs from that of a first independent infusion command, this process is identical to operations 107 and 108 of FIG. 38 described above, and thus, redundant descriptions thereof will be omitted.

In operation 1091, the controller 20 stores the received medical liquid infusion information and, based on the stored medical liquid infusion information, obtains a medical liquid infusion amount. The medical liquid infusion information may include an actual medical liquid infusion amount, which is the amount of medical liquid actually infused. Unlike in previous embodiments, the controller 20 does not need to calculate a medical liquid infusion rate, and directly obtains the medical liquid infusion amount.

In operation 1101, the controller 20 generates and displays a chart of the medical liquid infusion amount over time, based on the obtained medical liquid infusion amount. For example, the chart may be a line graph. In addition, a line graph generated in the first period Phase 1 may be indicated by, for example, a solid line. Referring to FIG. 53, unlike the charts of the previous embodiments, the vertical axis represents infusion amount (unit) rather than infusion rate, and the horizontal axis represents time (hour). From the values on the vertical axis of the solid line graph, the amount of medical liquid infused up to the current time, that is, the amount of insulin (U), may be confirmed. When an independent infusion command B with an infusion amount of 2.00 U is input, as exemplified in FIG. 52A, then according to FIG. 53, the user may confirm from the graph that 1.00 U of medical liquid has been infused up to the current time.

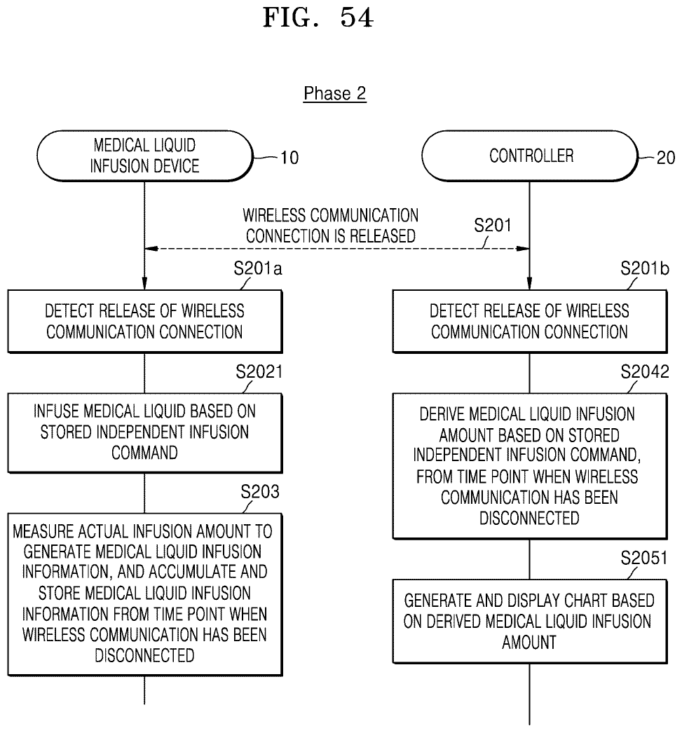

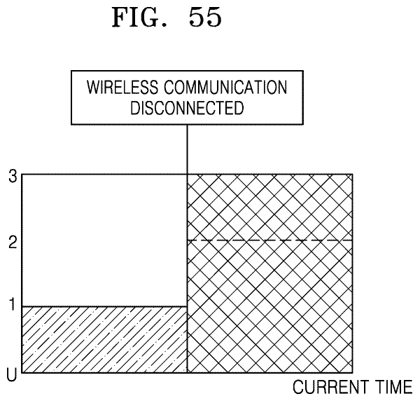

FIG. 54 is a flowchart illustrating a method of displaying medical liquid infusion in a second period Phase 2, according to yet another embodiment of the disclosure. FIG. 55 illustrates a medical liquid infusion information chart displayed on the controller 20 in the second period Phase 2. Here, the second period Phase 2 refers to a period when an event has occurred in the wireless communication connection between the medical liquid infusion device 10 and the controller 20. In other words, it refers to a period when the wireless communication connection between the medical liquid infusion device 10 and the controller 20 is released or disconnected.

Operations 201 to 201b are identical to operations 201 to 201b of FIG. 41 described above, and thus, redundant descriptions thereof will be omitted.

In operation 2021, similarly to operation 106, the medical liquid infusion device 10 infuses the medical liquid into the subject according to the infusion amount included in the stored independent infusion command B, and in operation 203, similarly to operation 107, when the medical liquid is infused, the medical liquid infusion device 10 measures the medical liquid infusion amount, generates medical liquid infusion information, and accumulates and stores the medical liquid infusion information. Here, the medical liquid infusion device 10 accumulates and stores, in chronological order, the medical liquid infusion information generated from the time point when the wireless communication event has occurred. The details of this process are similar to those of operation 203 of FIG. 41, and thus, redundant descriptions thereof will be omitted.

In operation 2042, after detecting the wireless communication-related event, the controller 20 derives an infusion amount based on the stored independent infusion command B, from the time point when the wireless communication has been disconnected. Because the independent infusion command B includes an infusion amount, the controller 20 may derive the infusion amount based on the stored independent infusion command B even without receiving medical liquid infusion information from the medical liquid infusion device 10 during the period the wireless communication connection is released.

In operation 2051, the controller 20 generates and displays a chart based on the derived infusion amount. For example, the chart may be a line graph. In addition, a line graph generated in the second period Phase 2 may be displayed to be distinguishable from the graph generated in the first period Phase 1, and may be indicated by, for example, a dotted line. Referring to FIG. 55, the vertical axis represents the infusion amount (U), and the horizontal axis represents time (hour). The dotted line graph indicates the infusion amount included in the independent infusion command B. That is, the user may confirm information about the infusion amount that needs to be ultimately administered, even during the second period Phase 2.

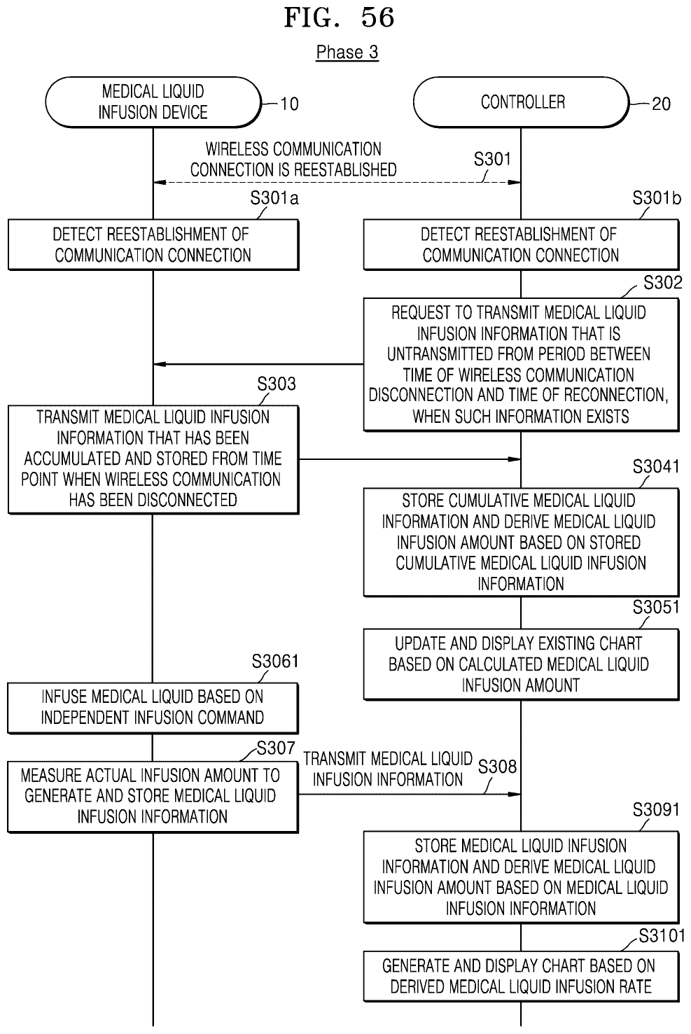

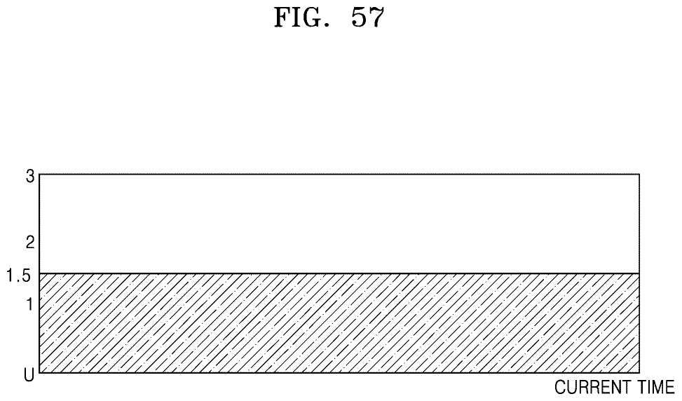

FIG. 56 is a flowchart illustrating a method of displaying medical liquid infusion in a third period Phase 3, according to yet another embodiment of the disclosure. FIG. 57 illustrates a medical liquid infusion information chart displayed on the controller 20 in the third period Phase 3. Here, the third period Phase 3 refers to a period when an event that had occurred in the wireless communication connection between the medical liquid infusion device 10 and the controller 20 has been resolved. In other words, it refers to a period when the time point when the wireless communication connection between the medical liquid infusion device 10 and the controller 20 is reestablished.

When the event that has occurred in relation to wireless communication is resolved, the medical liquid infusion device 10 transmits, to the controller 20, cumulative medical liquid infusion information, which has been accumulated and stored from the time point when the wireless communication has been disconnected. Operations 301 to 303 are identical to operations 301 to 303 of FIG. 43 described above, and thus, redundant descriptions thereof will be omitted.

In operation 3041, the controller 20 stores the received cumulative medical liquid information and calculates a medical liquid infusion amount based on the stored cumulative medical liquid infusion information. The cumulative medical liquid infusion information may include the amount of medical liquid actually infused that could not be transmitted to the controller 20 from the time point when the wireless communication connection has been released until the wireless communication connection is reestablished. The controller 20 derives a medical liquid infusion amount from the amount of medical liquid actually infused.

In operation 3051, the controller 20 may update the chart generated in operation 2051, based on the derived medical liquid infusion amount. Here, the chart generated in operation 2051 refers to a chart indicating the infusion amount included in the independent infusion command B during the period of communication disconnection. For example, the chart updated in operation 3051 may be a line graph. A line graph generated in the third period Phase 3 may be indicated by a solid line, similar to the line graph generated in the first period Phase 1. In other words, the graph generated in the third period is distinguishable from that generated in the second period, but may be represented in the same manner as that of the first period.

The vertical axis of FIG. 57 represents the infusion amount (unit), and the horizontal axis represents time (hour). When an independent infusion command B with an infusion amount of 2.00 U is input, as exemplified in FIG. 52 (a), then according to FIG. 57, it may be confirmed that a total of 1.50 U of medical liquid has been infused up to the current time when the communication connection is reestablished. In addition, although not illustrated, it may be confirmed that, after a predetermined further time period has elapsed, a total of 2.00 U of medical liquid, which is specified in the independent infusion command B, has ultimately been infused.

In this manner, after a wireless communication-related event has been resolved, the controller 20 may receive accumulated medical liquid infusion information from the medical liquid infusion device 10 and, by updating information provided to the user during the period of communication disconnection to reflect the actual medical liquid infusion amount based on the accumulated medical liquid infusion information, provide accurate medical liquid infusion information to the user.

In operations 3061 to 3101, similarly to operations 1051 to 1101 of FIG. 51, a chart may be displayed as a solid line graph, similar to that generated in the first period, and this is identical to the above description, and thus, redundant descriptions thereof will be omitted.

The above-described method may be applied to various wearable healthcare devices that utilize wireless communication, enabling a user to confirm the operating state of the wearable healthcare device even when a wireless communication-related event occurs. For example, the wearable healthcare device may be an insulin patch or a wearable insulin pump used to infuse a medical liquid into the user, and this method may be utilized in various digital healthcare industries.

Various embodiments of the disclosure may be implemented as software (e.g., a program) including one or more instructions stored in a machine-readable storage medium. For example, a processor of a machine device may call at least one instruction among one or more stored instructions from a storage medium and execute the same. This enables the machine to be operated to perform at least one function according to the at least one instruction invoked. The one or more instructions may include code generated by a compiler or code executable by an interpreter. The machine-readable storage medium may be provided in the form of a non-transitory storage medium. Here, ‘non-transitory’ only means that the storage medium is a tangible device and does not contain signals (e.g., electromagnetic waves), and this term does not distinguish between a case where data is stored semi-permanently in a storage medium and a case where data is temporarily stored.

According to an embodiment, the method according to various embodiments of the disclosure may be included and provided in a computer program product. Computer program products may be traded between sellers and buyers as commodities. A computer program product is distributed in the form of a machine-readable storage medium (e.g., compact disc read only memory (CD-ROM)), or through an application store (e.g., Play Store™) or between two user devices directly or online (e.g., downloaded or uploaded). In the case of online distribution, at least part of a computer program product may be temporarily stored or temporarily created in a machine-readable storage medium such as a server of a manufacturer, an application store server, or a memory of a relay server.

Also, in this specification, a “unit” may refer to a hardware component such as a processor or a circuit, and/or a software component executed by the hardware component such as a processor.

The scope of the present embodiments is indicated by the appended claims rather than the detailed description above, and should be construed as including all changes or modifications derived from the meaning and scope of the claims and equivalent concepts thereof.

1. A method of controlling a medical liquid infusion device, the method comprising:
establishing, by a medical liquid infusion device, which is configured to be attached to a subject and infuse a medical liquid, and a controller, a connection for first wireless communication;
exchanging, by the controller and the medical liquid infusion device, an encryption key through second wireless communication;
determining, by the controller, whether a received signal is an infusion signal;
based on a result of the determining indicating that the signal is the infusion signal, encrypting, by the controller, the signal by using the encryption key;
transmitting, by the controller, the encrypted signal to the medical liquid infusion device through the second wireless communication;
decrypting, by the medical liquid infusion device, the encrypted signal by using the encryption key; and
infusing, by the medical liquid infusion device, a medical liquid according to the decrypted signal.

2. The method of claim 1, wherein the first wireless communication is established when the medical liquid infusion device and the controller are within a first distance and after the medical liquid infusion device and the controller undergo a pairing process, and the second wireless communication is usable without a pairing process when the medical liquid infusion device and the controller are within a second distance that is shorter than the first distance.

3. The method of claim 1, wherein the first wireless communication is Bluetooth or Bluetooth Low Energy, and
the second wireless communication is near-field communication (NFC).

4. The method of claim 1, wherein the infusion signal is an infusion signal that is input from an integrated management server connected to the controller via a network, and then transmitted to the controller.

5. The method of claim 1, wherein the infusion signal is an infusion signal that is directly input through an input/output module of the controller, and controls such that the medical liquid is immediately infused into the subject.

6. The method of claim 1, wherein the determining of whether the signal received by the controller is the infusion signal comprises:
based on the result of the determining indicating that the signal is not the infusion signal, transmitting, by the controller, the signal to the medical liquid infusion device through the first wireless communication; and
controlling, by the medical liquid infusion device, the medical liquid infusion device according to the received signal.

7. The method of claim 6, wherein cases in which the signal is not the infusion signal comprise cases in which the signal is a general control signal for controlling the medical liquid infusion device or is data.
